(19) United States
(12) Patent Application Publication
(10) Pub. No.: US 2010/0283040 A1
(43) Pub. Date: Nov. 11, 2010

(54) SELENOPHENES AND SELENOPHENE-BASED POLYMERS, THEIR PREPARATION AND USES THEREOF

(76) Inventors: Michael Bendikov, Rechovot (IL); Asit Patra, Rehovot (IL); Yair-Haim Wijsboom, Rehovot (IL); Mao Li, Rehovot (IL); Yana Sheynin, Rehovot (IL); Natalia Zamoshchik, Rehovot (IL); Sanjio S. Zade, Rehovot (IL)

Correspondence Address: Pearl Cohen Zedek Latzer, LLP, 1500 Broadway, 12th Floor, New York, NY, 10036, US

(21) Appl. No.: 12/677,364

(22) PCT Filed: Sep. 10, 2008
(86) PCT No.: PCT/IL08/01212

§ 371 Date: Jun. 14, 2010

Related U.S. Application Data
(60) Provisional application No. 60/935,978, filed on Sep. 10, 2007.
(60) Provisional application No. 61/129,444, filed on Jun. 26, 2008.

(51) Int. Cl. H01L 51/52; C07D 345/00; C07D 517/04; C08G 79/00; H01B 1/12

(52) U.S. Cl. 257/40; 540/1; 549/362; 549/350; 549/15; 549/11; 549/359; 528/403; 252/500; 257/E51.018

(57) ABSTRACT

This invention is directed to selenophene compounds, selenophene-based polymers (polyselenophene), processes for the preparation of the same and uses thereof. The polyselenophenes of this invention have high conductivity and can be used as electrodes in various devices such as in electrochromic devices, batteries, solar cells, optical amplifiers, organic light emitting diodes, and the like.

### FIELD OF THE INVENTION

This invention is directed to selenophene compounds, selenophene-based polymers (polyselenophene), processes for the preparation of the same and uses thereof. The polyselenophenes of this invention have high conductivity and can be used as electrodes in various devices such as in electrochromic devices, batteries, solar cells, optical amplifiers, organic light emitting diodes, and the like.

### BACKGROUND OF THE INVENTION

With continued developments resulting in the thinning and miniaturization of electronic devices, the development of novel conducting materials compatible with such devices is required. In this arena, extensive investigations on conductive polymers have resulted in identification of numerous potential applications. For example, polyacetylenes were investigated for possible availability as electrode materials in secondary batteries since they show conductivities as high as 10² to 10³ S·cm⁻¹ when doped with iodine or arsenic pentafluoride. These polymers also displayed excellent charge-discharge characteristics. Use of polyacetylenes in solar batteries was studied because of the polymers light absorption characteristics which are close to those of sunlight.

Various conductive organic polymers based on heterocyclic ring systems are also known. Among these are sulfur-containing heterocyclic polymers derived from heterocyclic systems which include, but are not limited to, thiophene-dibenzothiophene-bithiophene-substituted thiophenes-2-bromo-8-hydroxy-5,5-dioxodibenzothiophene- and tetrathiapentalene.

Among the heterocycle-based conductive organic polymers, polythiophenes have been studied as general conductive materials, battery electrode materials and electrochromic materials. The latter takes advantages of changes in polymer color when the polymer is doped. In one particular study, 2,2′-biphenyl was polymerized under electrochemical conditions generating a polymer which changed color from blue to red, depending upon the polymer oxidation state.

Since the development of conductive organic polymers continues to hold promise, poly(heterocycles) receive attention due to their ease of preparation and their enhanced stability to atmosperic conditions compared to the analogous polyacetylenes and polyphenylenes. Furthermore, these polymers can be advantageously prepared as thin conductive films, thus find application as components of electronic devices, with conductive organic polymers being useful for stabilizing semiconductor surfaces, as well.

There is a continuing need for new conducting polymers for use in electrochromic displays, batteries, solar cells, optical amplifiers, organic light emitting diodes, and the like

### SUMMARY OF THE INVENTION

In one embodiment, the present invention provides a compound represented by the structure of formula (I):

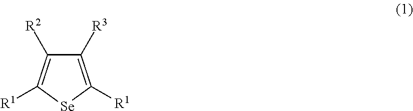

wherein: R¹ is H, F, Cl, Br, I, SH, OSO₂CH₃ or OSO₂CF₃;

R² is H, F, Cl, Br, I, C₁-C₆alkyl, Y—H or Y—(C₁-C₆ alkyl) wherein if R³ is H then R² is not C₁-C₆alkyl;

R³ is H, F, Cl, Br, I, C₁-C₆ alkyl, Z—H or Z—(C₁-C₆ alkyl) wherein if R² is H then R³ is not C₁-C₆alkyl;

or

R² and R³ combine to form a 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];

or

R² and R³ combine to form Y-ring-Z having the following structure;

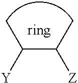

wherein said ring of Y-ring-Z is a cycloalkyl, heterocycloalkyl or aryl ring optionally substituted by 1-3 groups comprising halide, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];

to R⁴ is C₁-C₆ alkyl;

R⁵ is C₁-C₆ alkyl;

Y is O, S, Se, PH, NR⁶ or C(R⁷)(R⁸);

Z is O, S, Se, PH, NR⁹ or C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl;

R¹¹ is H, CN, C₁-C₆ alkyl, OH, SH, NH₂ or aryl;

wherein

if R¹ is H, then R² and R³ are not OMe, do not form an unsubstituted [1,4] dioxane ring; and do not form a 5 membered unsaturated heterocyclic ring comprising Se or S.

In one embodiment, the present invention provides a polymer represented by formula (25):

-[(A)ₒ-(B)ₚ—(C)_{q}]ᵣ— (25)

wherein A, B and C may be positioned in any order relative to one another such that A, B and C are either uniformly distributed as blocks throughout the polymer or A, B and C are randomly distributed throughout the polymer and wherein A is a monomer unit represented by the structure:

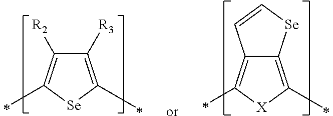

B is a monomer unit comprised of a monocyclic or bicyclic aryl or heteroaryl group wherein said monocyclic or bicyclic aryl or heteroaryl group is optionally substituted with 1-3 groups comprising CN, COOH, C₁-C₁₈ alkyl, OH, O—(C₁-C₁₈ alkyl), SH, S—(C₁-C₁₈ alkyl), NH₂, NH—(C₁-C₁₈ alkyl), or N(C₁-C₁₈ alkyl)₂;

C is a monomer unit comprised of a substituted or unsubstituted vinyl or acetylene group;

o is an integer from 1-10,000;

p is an integer from 0-10,000;

q is an integer from 0-10,000;

r is an integer from 2-10,000;

R² is H, F, Cl, Br, I, C₁-C₆alkyl, S, O, NH, Y—H or Y—(C₁-C₆ alkyl) wherein if R³ is H then R² is not C₁-C₆ alkyl;

R³ is H, F, Cl, Br, I, C₁-C₆ alkyl, Z—H or Z—(C₁-C₆ alkyl) wherein if R² is H then R³ is not C₁-C₆ alkyl;

or

R² and R³ combine to form a 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];

or

R² and R³ combine to form Y-ring-Z having the following structure;

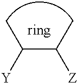

wherein said ring of Y-ring-Z is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];

R⁴ is C₁-C₆ alkyl;

R⁵ is C₁-C₆ alkyl;

Y is O, S, Se, NR⁶ and C(R⁷)(R⁸);

Z is O, S, Se, NR⁹ and C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl; and

R¹¹ is H, CN, C₁-C₆ alkyl, OH, SH, NH₂ or aryl;

X is S, Se.

In one embodiment, the present invention provides a process for preparing a compound represented by the structure of formula (1):

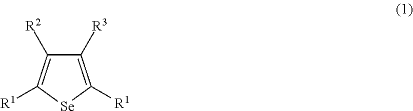

wherein: R¹ is H, F, Cl, Br, I, SH, OSO₂CH₃ or OSO₂CF₃;

R² is H, F, Cl, Br, I, C₁-C₆alkyl, S, O, NH, Y—H or Y—(C₁-C₆ alkyl) wherein if R³ is H then R² is not C₁-C₆ alkyl;

R³ is H, F, Cl, Br, I, C₁-C₆ alkyl, Z—H or Z—(C₁-C₆ alkyl) wherein if R² is H then R³ is not C₁-C₆ alkyl;

or

R² and R³ combine to form a substituted or unsubstituted 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];

or

R² and R³ combine to form Y-ring-Z having the following structure;

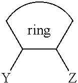

wherein said ring of Y-ring-Z is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];

R⁴ is C₁-C₆ alkyl;

R⁵ is C₁-C₆ alkyl;

Y is O, S, NR⁶ and C(R⁷)(R⁸);

Z is O, S, NR⁹ and C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl;

R¹¹ is H, CN, C₁-C₆ alkyl, OH, SH, NH₂ or aryl;

said process comprising the steps of:

a) reacting 3,4-dimethoxyselenophene with a nucleophile to yield 3,4-substituted-selenophene:

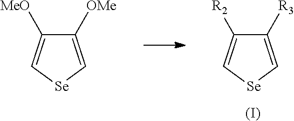

b) optionally brominating 3,4-substituted-selenophene (I) of step (a), obtaining 2,5 dibromoselenophene:

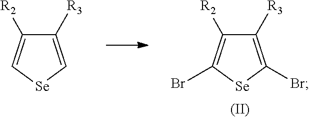

and

c) optionally substituting the bromide of compound (II) of step (b) with a nucleophile, obtaining the compound of formula (1).

In one embodiment, the present invention provides a process for preparing a polymer of formula (25):

-[(A)ₒ-(B)ₚ—(C)_{q}]ᵣ— (25)

wherein A, B and C may be positioned in any order relative to one another such that A, B and C are either uniformly distributed as blocks throughout the polymer or A, B and C are randomly distributed throughout the polymer and wherein:

A is a monomer unit represented by the structure:

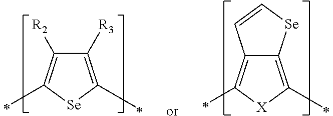

B is a monomer unit comprised of a monocyclic or bicyclic aryl or heteroaryl group wherein said monocyclic or bicyclic aryl or heteroaryl group is optionally substituted with 1-3 groups comprising CN, COOH, C₁-C₁₈ alkyl, OH, O—(C₁-C₁₈ alkyl), SH, S—(C₁-C₁₈ alkyl), NH₂, NH—(C₁-C₁₈ alkyl), or N(C₁-C₁₈ alkyl)₂;

C is a monomer unit comprised of a substituted or unsubstituted vinyl or acetylene group;

o is an integer from 1-10,000;

p is an integer from 0-10,000;

q is an integer from 0-10,000;

r is an integer from 2-10,000;

R² is H, F, Cl, Br, I, C₁-C₆alkyl, S, O, NH, Y—H and Y—(C₁-C₆ alkyl) wherein if R³ is H then R² is not C₁-C₆ alkyl;

R³ is H, F, Cl, Br, I, C₁-C₆ alkyl, Z—H and Z—(C₁-C₆ alkyl) wherein if R² is H then R³ is not C₁-C₆ alkyl;

or

R² and R³ combine to form a 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];

or

R² and R³ combine to form Y-ring-Z having the following structure;

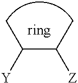

wherein said ring of Y-ring-Z is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];

R⁴ is C₁-C₆ alkyl;

R⁵ is C₁-C₆ alkyl;

Y is O, S, Se, NR⁶ or C(R⁷)(R⁸);

Z is O, S, Se, NR⁹ or C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl; and

R¹¹ is H, CN, C₁-C₆ alkyl, OH, SH, NH₂ or aryl;

X is S or Se;

said process comprises the step of polymerizing a 2,5-dibromoselenophene or 2,5-diiodoselenophene monomer unit of A with monomer unit B, monomer unit C or any combination thereof, in the presence of Ni(COD)₂ or by heating said monomer units to a temperature range of between 20-150° C.; or said process comprises the step of polymerizing a selenophene monomer unit of A, with monomer unit B, monomer unit C or any combination thereof, in the presence of FeCl₃ or polymerizing said monomers electrochemically, wherein position 2, 5 of said selenophene monomer unit of A are hydrogens.

In one embodiment, the present invention provides an organic light-emitting device, comprising: a first electrode; a second electrode; an emitting layer interposed between the first electrode and the second electrode; and at least one of a hole transporting layer and a hole injecting layer interposed between the emitting layer and the first electrode, said at least one of the hole transporting layer and the hole injecting layer obtained from a conducting polymer.

In one embodiment, the polymers of this invention are used in electrochromic devices wherein said polymers have high coloration efficiencies.

In one embodiment, the present invention provides a radialene compound of formula (46):

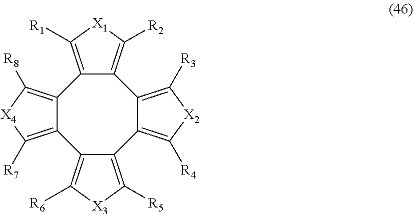

wherein: R¹, R², R³, R⁴, R⁵, R⁶, R⁷ or R⁸ are independently H, F, Cl, Br, I, CN, OH, SH, NH₂, O—(C₁-C₆ alkyl), S—(C₁-C₆ alkyl), NH(C₁-C₆ alkyl), N(R⁹)(R¹⁰), NHC(O)(C₁-C₆ alkyl) and N[(C₁-C₆ alkyl)][C(O)(C₁-C₆ alkyl)] or C(O)OR¹¹;

R⁹ is C₁-C₆ alkyl;

R¹⁰ is C₁-C₆ alkyl;

R¹¹ is C₁-C₆ alkyl; and

X¹, X², X³ or X⁴ are independently O, S, Se, Te, NH or PH;

wherein X¹, X², X³ and X⁴ are not S.

In one embodiment, the present invention provides a radialene compound of formula (47):

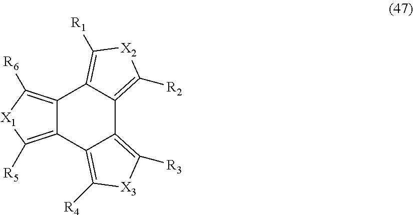

wherein: R¹, R², R³, R⁴, R⁵ or R⁶ are independently H, F, Cl, Br, I, CN, OH, SH, NH₂, O—(C₁-C₆ alkyl), S—(C₁-C₆ alkyl), NH(C₁-C₆ alkyl), N(R⁷)(R⁸), NHC(O)(C₁-C₆ alkyl) and N[(C₁-C₆ alkyl)][C(O)(C₁-C₆ alkyl)] or C(O)OR⁹;

R⁷ is C₁-C₆ alkyl;

R⁸ is C₁-C₆ alkyl;

R⁹ is C₁-C₆ alkyl;

X¹, X² or X³ are independently O, S, Se, Te, NH or PH;

wherein X¹, X² and X³ are not S.

In one embodiment, this invention is directed to polyselenophene dispersion comprising positively charged polyselenophene and an anion, wherein said polyselenophene is of formula (25) and said anion is tosylate, acrylate, maleate, sulfonate, p-toluenesulfate, 4-ethlybenzenesulfonate, camphor-sulfonate, tetradecyl-sulfonate, dodecyl-sulfonate, methane-sulfonate, naphthalene sulfonate, triflate, or any combination thereof; or an anion of polyacrylic acid, polymethacrylic acid, polymaleic acid, polystyrene sulfonic acid, polyvinyl sulfonic acid combination thereof.

In one embodiment, this invention is directed to polyselenophene dispersion comprising positively charged polyselenophene and an anion, wherein said polyselenophene is of formula (53) and said anion is tosylate, acrylate, maleate, sulfonate, p-toluenesulfate, 4-ethlybenzenesulfonate, camphor-sulfonate, tetradecyl-sulfonate, dodecyl-sulfonate, methane-sulfonate, naphthalene sulfonate, triflate, or any combination thereof; or an anion of polyacrylic acid, polymethacrylic acid, polymaleic acid, polystyrene sulfonic acid, polyvinyl sulfonic acid combination thereof.

In another embodiment, the polyselenophene dispersion is in water or in alcohol.

In one embodiment, this invention provides a transparent conducting electrode comprising the selenophene dispersion of this invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts a synthetic scheme for the preparation of compound (3).

FIG. 2 depicts a synthetic scheme for the polymerization of a selenophene monomer unit or a 2,5-dibromoselenophene monomer unit or 2,5-diiodoselenophene monomer for the preparation of poly(3,4-ethylenedioxyselenophene).

FIG. 3 depicts a synthetic scheme for the preparation of compound (24).

FIG. 4 depicts various selenophenes and selenophene polymers accessible via modifications of 3,4-dimethoxyselenophene.

FIG. 5 depicts a synthetic scheme for the preparation of selenophene dimers and oligomers.

FIG. 6 depicts selenophene-thiophene co-polymers.

FIG. 7 depicts a synthetic scheme for the preparation of selenophene and thiophene-containing radiallenes.

FIG. 8 depicts the appearance of a 2,5-dibromoselonophene monomer unit compared to the appearance of its black doped polymer formed on heating of the monomer unit.

FIG. 9 depicts a crystal packing diagram of a 2,5-dibromoselenophene monomer unit as indicated by x-ray crystallographic analysis where bromine atoms are blue, selenium atoms are pink, carbon atoms are gray, oxygen atoms are green and hydrogen atoms are white.

FIG. 10 depicts a first scan of cyclic voltammetry analysis of 3,4-ethylenedioxyselenophene (EDOS) monomer in acetonitrile +0.1M (Bu₄N)ClO₄ on Pt electrode. Repeating such scan lead to the formation of polymer film. X-axis is in volts and Y-axis is in microamperes.

FIG. 11 depicts a cyclic voltammetry analysis of 3,4-ethylenedioxyselenophene (EDOS) monomer in acetonitrile +0.1M (Bu₄N)ClO₄ on Pt electrode. Shown is the multisweep electropolymerization of EDOS monomer at 50 mV/s, Fc/Fc+=0.37 V. X-axis is in volts and Y-axis is in microamperes.

FIG. 12 depicts a cyclic voltammetry analysis of poly(3,4-ethylnedioxyselenophenes) (PEDOS) in monomer free acetonitrile +0.1M (Bu₄N)ClO₄ on Pt electrode and formed via elecropolymerization. X-axis is in volts and Y-axis is in microamperes.

FIG. 13 depicts a cyclic voltammetry analysis of CV of poly(3,4-ethylnedioxyselenophenes) (PEDOS) in monomer free acetonitrile +0.1M (Bu₄N)ClO₄ on Pt electrode and formed via solid-state polymerization from 2,5-dibromo-3,4-ethylene-dioxythiophene DBEDOS, Fc/Fc+=0.37 V. X-axis is in volts and Y-axis is in microamperes.

FIG. 14 depicts an optoelectrochemical spectra for poly(3,4-ethylnedioxyselenophenes) (PEDOS) as a function of applied potential between −1.3 and +0.6 V in PC +0.1M TBAPC. X-axis is in nanometer and Y-axis is in absolute units.

FIG. 15 depicts a temperature dependant conductivity measurements for a polyselenophene. X-axis is Kelvin and Y-axis is Ohm×cm

FIG. 16 depicts (a) Multisweep electropolymerization of the monomer on Pt electrode in acetonitrile to produce the all selenium polymer (35), X-axis is in volts and Y-axis is in microamperes (b) CV of produced (35) in monomer free acetonitrile, X-axis is in volts and Y-axis is in microamperes (c) spectroelectrochemistry data for the all selenium polymer (35). X-axis is in nanometer and Y-axis is in absolute units.

FIG. 17 depicts the electrochemical polymerization of all selenium (24) compound. X-axis is in volts and Y-axis is in microamperes.

FIG. 18 depicts X-ray structure of compound (47), wherein R¹-R⁶ are H (top: ORTEP diagrams; bottom: packing pattern) of compounds S-radialene (left) (two molecules are shown in ORTEP diagram) and Se-radialene (right). In both structures stacking molecules are positioned on the same translation axis (b axis for S-radialene and c axis for Se-Radialene).

FIG. 19 depicts a UV-VIS absorption spectra of compounds selected radialene compounds.

FIG. 20 depicts a cyclic voltametry of selected radialene compounds. (acetonitrile +0.1M (Bu₄N)ClO₄, Pt working electrode, scan rate 10 mV/s, vs. Fc/Fc⁺=0.37 V).

FIG. 21 depicts a spectroelectrochemistry of poly(seleno[3,4-b]thiophene, polymer (53) comprising monomer A of formula (37).

FIG. 22 depicts contrast ratio and coloration efficiency data reported in the literature for PEDOT and its derivatives. PEDOT, PPropOT, PPropOT-Me₂, PPropOT-Et₂, PPropOT-Bz₂, P(BEDOT-MEHB); which are known compounds and poly(3,4-ethylnedioxyselenophenes) (PEDOS) and poly(hexane-3,4-ethylnedioxyselenophenes) (PEDOS-C₆) which are the compounds of this invention.

FIG. 23 depicts the simultaneous monitoring of (a) transmittance and (b) switching current for poly(3,4-ethylnedioxyselenophenes) (PEDOS) film monitored at 666 nm, when it was switched between its neutral (−0.9 V vs. Ag/AgCl) and oxidized (+0.5 V vs. Ag/AgCl) states with 5 s intervals.

FIG. 24 depicts the switching studies of (a) percent transmittance monitored at 763 nm and (b) current for poly(hexane-3,4-ethylnedioxyselenophenes) (PEDOS-C₆) film, when it was stepped between its neutral (−0.9 V) and oxidized (+0.5 V) states with 3 s intervals.

FIG. 25 depicts transmittance values (at 763 nm) of poly(hexane-3,4-ethylnedioxyselenophenes) (PEDOS-C₆) films on ITO-coated glass as a function of film deposition charge as obtained by switching the voltage between −0.9 V (neutral colored state) and +0.5 V vs. Ag/AgCl (oxidized bleached state) with 3 s intervals. The contrast ratio is a difference between a percentage transmittance (% T) at −0.9 V and % T at +0.5 V.

FIG. 26 depicts the switching time for poly(hexane-3,4-ethylnedioxyselenophenes) (PEDOS-C₆) film monitored at 763 nm.

FIG. 27 depicts the long term stability of the transmittance values of poly(hexane-3,4-ethylnedioxyselenophenes) (PEDOS-C₆) stepped between its neutral (−0.9 V) and oxidized (+0.5 V) states with 3 s intervals and switched through 10000 switching cycles.

FIG. 28 depicts cyclic voltammetry of poly(alkyl-3,4-ethylnedioxyselenophenes) (PEDOS-Cₙ) films, which were prepared at a constant potential using an electrodeposited charge of 0.05 C at scan rate of 20 mV/s in 0.1 M LiBF₄/PC.

FIG. 29 depicts spectroelectrochemical spectra for poly(alkyl-3,4-ethylnedioxyselenophenes) (PEDOS-Cₙ) films (n=0 (a), 2 (b), 4 (c), 6 (d), 6(c) (e), 8(f), and 12 (g)) prepared on ITO-coated glass at applied potentials of (a) −0.9, (b) −0.7, (c) −0.5, (d) −0.3, (e) −0.1, (f) 0, (g) 0.1, (h) 0.3 V, (i) 0.5 V vs. Ag/AgCl.

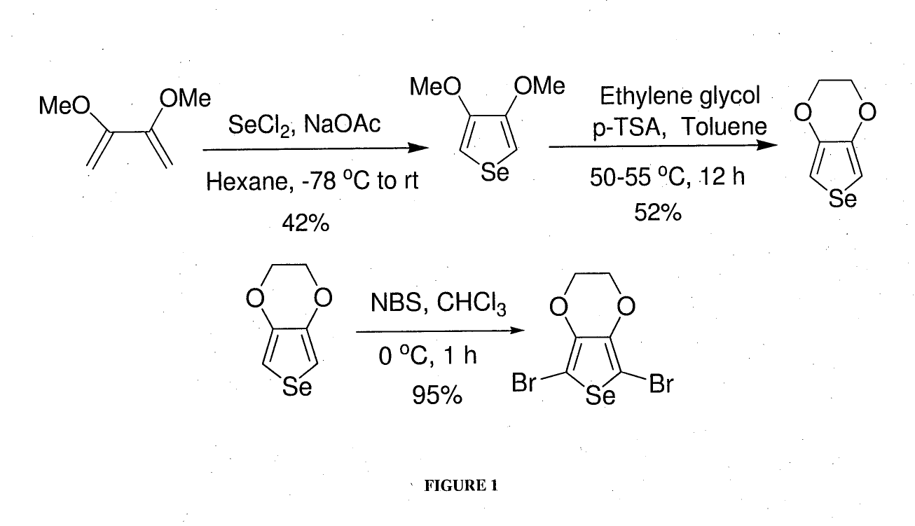

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

This invention provides, in one embodiment, selenophene based polymers, which are useful in some embodiments as electrodes in various devices and in some embodiments, substituted polyselenophenes and their use in electrochromic displays, batteries, solar cells, optical amplifiers, organic light emitting diodes, and the like.

In some embodiments, this invention provides synthetic processes of preparation of the selenophene compounds of this invention. In some embodiments, this invention provides synthetic processes of preparation of the selenophenes-based polymers of this invention.

In one embodiment, this invention provides a compound represented by the structure of formula (1):

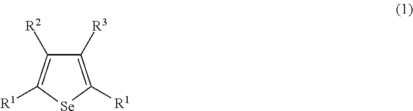

wherein: R¹ is H, F, Cl, Br, I, SH, OSO₂CH₃ or OSO₂CF₃;

R² is H, F, Cl, Br, I, C₁-C₆alkyl, Y—H or Y—(C₁-C₆ alkyl) wherein if R³ is H then R² is not C₁-C₆alkyl;

R³ is H, F, Cl, Br, I, C₁-C₆ alkyl, Z—H or Z—(C₁-C₆ alkyl) wherein if R² is H then R³ is not C₁-C₆alkyl;

or

R² and R³ combine to form a 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];

or

R² and R³ combine to form Y-ring-Z having the following structure;

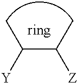

wherein said ring of Y-ring-Z is a cycloalkyl, heterocycloalkyl or aryl ring optionally substituted by 1-3 groups comprising halide, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];

R⁴ is C₁-C₆ alkyl;

R⁵ is C₁-C₆ alkyl;

Y is O, S, Se, PH, NR⁶ or C(R⁷)(R⁸);

Z is O, S, Se, PH, NR⁹ or C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl;

R¹¹ is H, CN, C₁-C₆ alkyl, OH, SH, NH₂ or aryl;

wherein if R¹ is H, then R² and R³ are not OMe, do not form an unsubstituted [1,4] dioxane ring; and do not form a 5 membered unsaturated heterocyclic ring comprising Se or S.

In another embodiment, R¹ of compound of formula (1) is H. In another embodiment, R¹ of compound of formula (1) is Br. In another embodiment, R¹ of compound of formula (1) is Cl. In another embodiment, R¹ of compound of formula (1) is I. In another embodiment, R¹ of compound of formula (1) is SH. In another embodiment, R¹ is Br, R² is Y—(C₁-C₆ alkyl), R³ is Z—(C₁-C₆ alkyl), Y is O and Z is O. In another embodiment, R¹ is I, R² is Y—(C₁-C₆ alkyl), R³ is Z—(C₁-C₆ alkyl), Y is O and Z is O. In another embodiment, R² is Y—CH₃ and R³ is Z—CH₃.

In one embodiment, R² and R³ of compound of formula 1, combine to form Y-ring-Z having the following structure;

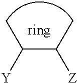

In another embodiment, Y and Z are attached to an aromatic ring on adjacent atoms. In another embodiment, Y and Z are attached to an aliphatic ring on adjacent atoms. In another embodiment, said ring is cyclohexane. In another embodiment, said ring is cyclopentane. In another embodiment, said ring is benzene. In another embodiment, said ring is naphthalene. In another embodiment, said ring is piperazine. In another embodiment, said ring is quinoline.

In another embodiment, the compound of formula (1) is represented by the structure of formula ((2):

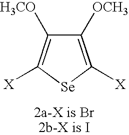

In another embodiment, R² and R³ combine to form a substituted or unsubstituted 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)].

In another embodiment, R² and R³ of formula (1) combine to form a substituted or unsubstituted 4-8 membered ring wherein said ring is substituted by 1-3 substituents. In another embodiment, said 4-8 membered ring is cyclohexane. In another embodiment, said 4-8 membered ring is cyclohexene. In another embodiment, said 4-8 membered ring is cyclopentane. In another embodiment, said 4-8 membered ring is cyclopentene. In another embodiment, said 4-8 membered ring is pyrrole. In another embodiment, said 4-8 membered ring is pyrrolidine. In another embodiment, said 4-8 membered ring is substituted [1-4] dioxane. In another embodiment, said 4-8 membered ring is [1-4] dioxane substituted by C₁-C₁₂ alkyl. In another embodiment, said 4-8 membered ring is [1-4] dithiane. In another embodiment, said 4-8 membered ring is [1,4]-dioxepane. In another embodiment, said 4-8 membered ring is [1,4]dithiepane. In another embodiment, said 4-8 membered ring is [1,4]diselenane. In another embodiment, said 4-8 membered ring is [1,4] diselenepane. In another embodiment, said 4-8 membered ring is furane. In another embodiment, said 4-8 membered ring is pyridine. In another embodiment, said 4-8 membered ring is tetrahydrofurane. In another embodiment, said 4-8 membered ring is tetrahydrothiopyran. In another embodiment, said 4-8 membered ring is benzene. In another embodiment, said 4-8 membered ring is pyridine. In another embodiment, R² and R³ of formula (1) combine to form a substituted or unsubstituted 4-8 membered ring wherein said ring is substituted by 1-3 substituents. In another embodiment, said substituents comprise C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)], or any combination thereof.

In another embodiment, R¹ is Br and R² and R³ of formula (1) combine to form a dioxy substituted or unsubstituted 4-8 membered ring containing 0-3 double bonds. In another embodiment, R¹ is I and R² and R³ of formula (1) combine to form a dioxy substituted or unsubstituted 4-8 membered ring containing 0-3 double bonds.

In another embodiment the compound of formula (1) is represented by the structure of formula (3):

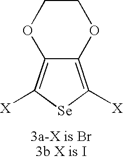

In another embodiment, the compound of formula (1) is represented by the structure of formula (4):

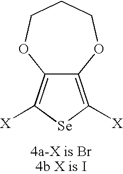

In another embodiment, the compound of formula (1) is represented by the structure of formula (5):

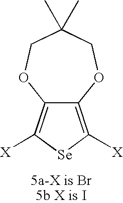

In another embodiment, the compound of formula (1) is represented by the structure of formula (6):

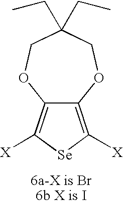

In another embodiment, the compound of formula (1) is represented by the structure of formula (7):

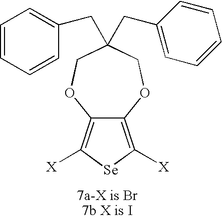

In another embodiment, R¹ is H and R² and R³ of formula (1) combine to form a dioxy substituted or unsubstituted 4-8 membered ring containing 0-3 double bonds; wherein; if R¹ is H, then R² and R³ do not form an unsubstituted [1,4] dioxane ring.

In another embodiment, the compound of formula (1) is represented by the structure of formula (8):

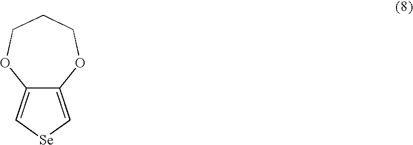

In another embodiment, the compound of formula (1) is represented by the structure of formula (9):

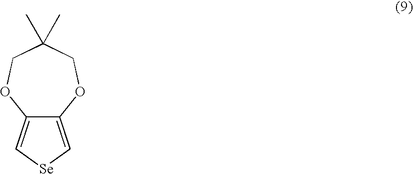

In another embodiment, the compound of formula (1) is represented by the structure of formula (10):

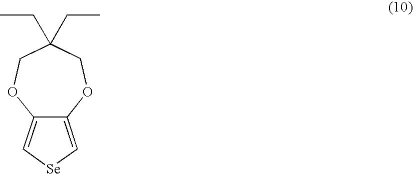

In another embodiment, the compound of formula (1) is represented by the structure of formula (11):

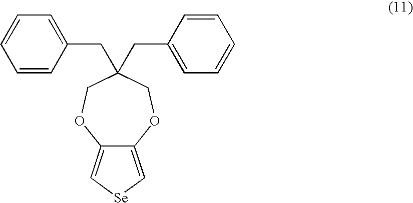

In another embodiment, the compound of formula (1) is represented by the structure of formula (12a):

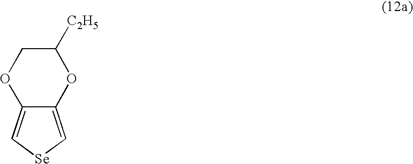

In another embodiment, the compound of formula (1) is represented by the structure of formula (12b):

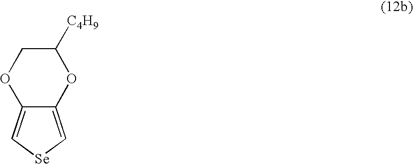

In another embodiment, the compound of formula (1) is represented by the structure of formula (12c):

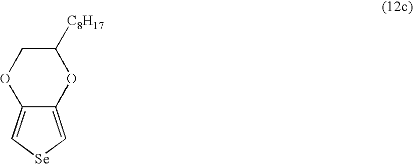

In another embodiment, the compound of formula (1) is represented by the structure of formula (12d):

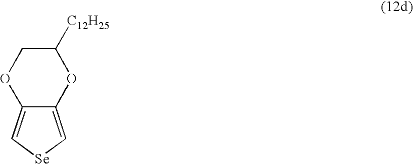

In another embodiment, the compound of formula (1) is represented by the structure of formula (12e) and 12(e)-chiral:

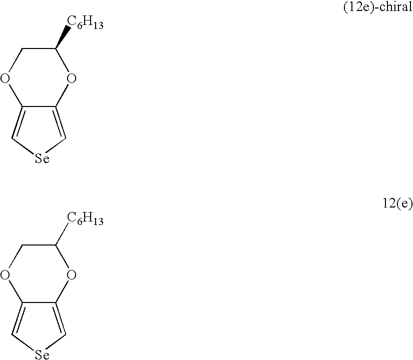

In another embodiment, the compound of formula (1) is represented by the structure of formula (12f):

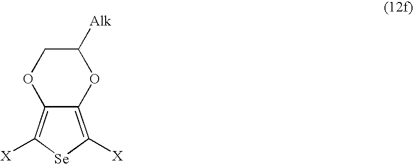

wherein Alk is C₁-C₁₂ alkyl; and X is H or halogen. In another embodiment, X is Br or I. In another embodiment X is H. In another embodiment Alk is ethyl, butyl, octyl, hexyl or dodecyl.

In another embodiment, the compound of formula 12(a)-12(f) is a racemic mixture. In another embodiment, the compound of formula 12(a)-12(f) is chiral.

In another embodiment, R¹ is Br and R² and R³ of formula (1) combine to form a dithio substituted or unsubstituted 4-8 membered ring containing 0-3 double bonds.

In another embodiment, the compound of formula (1) is represented by the structure of formula (13):

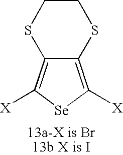

In another embodiment, the compound of formula (1) is represented by the structure of formula (14):

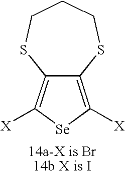

In another embodiment, the compound of formula (1) is represented by the structure of formula (15):

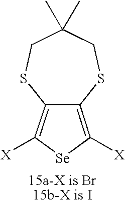

In another embodiment, the compound of formula (1) is represented by the structure of formula (16):

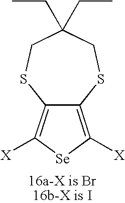

In another embodiment, the compound of formula (1) is represented by the structure of formula (17):

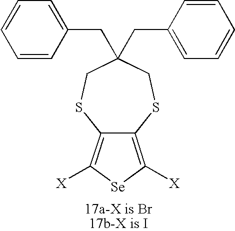

In another embodiment, R¹ is H and R² and R³ of formula (1) combine to form a dithio substituted or unsubstituted 4-8 membered ring containing 0-3 double bonds.

In another embodiment, the compound of formula (1) is represented by the structure of formula (18):

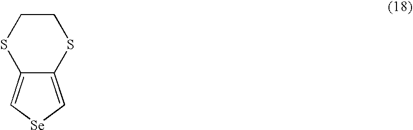

In another embodiment, the compound of formula (1) is represented by the structure of formula (19):

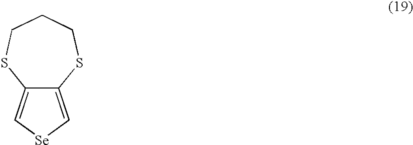

In another embodiment, the compound of formula (1) is represented by the structure of formula (20):

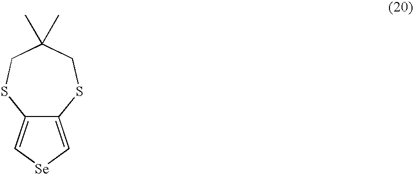

In another embodiment, the compound of formula (1) is represented by the structure of formula (21):

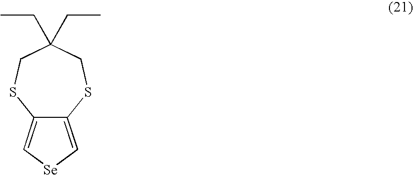

In another embodiment, the compound of formula (1) is represented by the structure of formula (22):

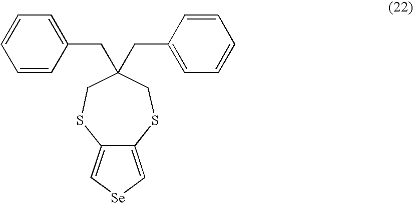

In another embodiment, the combination of R² and R³ is Y-phenyl-Z. In another embodiment, the compound of formula (1) is represented by the structure of formula (23):

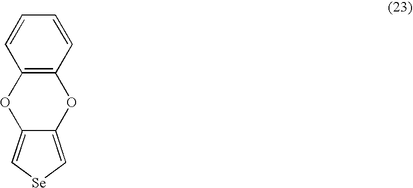

In another embodiment, the compound of formula (1) is represented by the structure of formula (24):

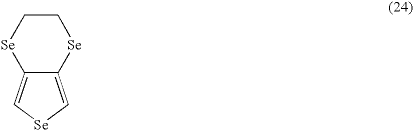

In another embodiment, the compound of formula (1) is represented by the structure of formula (42):

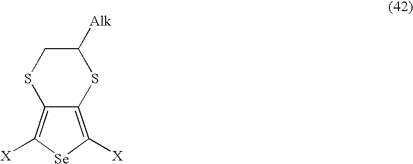

wherein Alk is a C₁-C₁₂ alkyl and X is H, or halogen. In another embodiment, the alkyl is ethyl, butyl, hexyl, octyl or dodecyl. In another embodiment, X is Br or I.

In another embodiment, the compound of formula (1) is represented by the structure of formula (43):

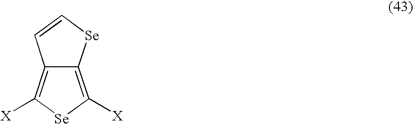

wherein X is H or halogen. In another embodiment X is bromine or iodine.

In another embodiment, the compound of formula (1) is represented by the structure of formula (44):

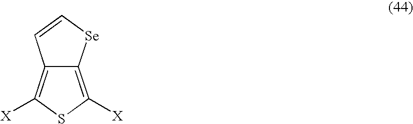

wherein X is H or an halogen. In another embodiment X is H. In another embodiment X is Br or I.

An “alkyl” group refers, in one embodiment, to a saturated aliphatic hydrocarbon, including straight-chain, branched-chain and cyclic alkyl groups. In one embodiment, the alkyl group has 1-12 carbons. In one embodiment, the alkyl group has 1-15 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl. In one embodiment, the alkyl group is CH₃.

A “haloalkyl” group refers to an alkyl group as defined above, which is substituted by one or more halogen atoms, in one embodiment by F, in another embodiment by Cl, in another embodiment by Br, in another embodiment by I.

A “cycloalkyl” group refers to a cyclic group having at least one saturated carbocyclic group which may be unsubstituted or substituted by one or more groups selected from halogen, haloalkyl, hydroxy, alkoxy, carbonyl, amido, alkylamido, dialkylamido, nitro, amino, alkylamino, dialkylamino, carboxy or thio or thioalkyl. Nonlimiting examples of cycloalkyl rings are cyclopropane, cyclobutane, cyclopentane, cyclohexane, cycloheptane, cyclooctane, and the like. In one embodiment, the cycloalkyl group is a 3-12 membered ring. In another embodiment, the cycloalkyl group is a 3-8 membered ring. In another embodiment, the cycloalkyl group comprises of 1-4 fused rings.

An “aryl” group refers to an aromatic group having at least one carbocyclic aromatic group or heterocyclic aromatic group, which may be unsubstituted or substituted by one or more groups selected from halogen, haloalkyl, hydroxy, alkoxy, carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, quinolyl, isoquinolyl, and the like. In one embodiment, the aryl group is a 3-12 membered ring. In another embodiment, the aryl group is a 3-8 membered ring. In another embodiment, the aryl group comprises 3-4 fused rings

A “hydroxyl” group refers to an OH group.

A “primary amide” group refers to an amide where the amide nitrogen is attached to two hydrogen atoms.

A “secondary amide” group refers to an amide where the amide nitrogen is simultaneously attached to a hydrogen atom and an alkyl group. In one embodiment, the alkyl group comprises of 1-12 carbon atoms. In another embodiment, the alkyl group is branched. In another embodiment, the alkyl group is unbranched. In another embodiment, the alkyl group comprises 1-6 carbon atoms. In another embodiment, the alkyl group is methyl. In another embodiment, the alkyl group is ethyl. In another embodiment, the alkyl group is isopropyl.

A “tertiary amide” group refers to an amide where the amide nitrogen is simultaneously attached to two alkyl groups. In one embodiment, the alkyl groups independently comprise 1-12 carbon atoms. In another embodiment, the alkyl groups are independently branched. In another embodiment, the alkyl groups are independently unbranched. In another embodiment, the alkyl groups independently comprise 1-6 carbon atoms. In another embodiment, the alkyl groups are methyl. In another embodiment, the alkyl groups are ethyl. In another embodiment, the alkyl groups are isopropyl.

An “ester” group refers to a carboxylic acid where the hydrogen of the carboxylic acid is replaced by an alkyl group or aryl group as defined hereinabove.

The term “halogen” refers to in one embodiment to F, in another embodiment to Cl, in another embodiment to Br, in another embodiment to I.

The term “ring” refers to a monocyclic or bicyclic aromatic or aliphatic ring system comprising 3-10 atoms. In one embodiment, said ring contains 0-4 heteroatoms selected from O, N and S. In another embodiment, said ring is optionally substituted with 0-3 groups. In another embodiment, said ring is cyclohexane. In another embodiment, said ring is cyclopentane. In another embodiment, said ring is benzene. In another embodiment, said ring is naphthalene. In another embodiment, said ring is piperazine. In another embodiment, said ring is quinoline.

A “heterocycle” group refers, in one embodiment, to a ring structure comprising in addition to carbon atoms, sulfur, oxygen, nitrogen or any combination thereof, as part of the ring. In another embodiment the heterocycle is a 3-12 membered ring. In another embodiment the heterocycle is a 6 membered ring. In another embodiment the heterocycle is a 5-7 membered ring. In another embodiment the heterocycle is a 4-8 membered ring. In another embodiment, the heterocycle group may be unsubstituted or substituted by a halogen, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the heterocycle ring may be fused to another saturated or unsaturated cycloalkyl or heterocyclic 3-8 membered ring. In another embodiment, the heterocyclic ring is a saturated ring. In another embodiment, the heterocyclic ring is an unsaturated ring.

The term “substituted” refers to substitution of one or more hydrogens with non-hydrogen groups. Non-limiting examples of non-hydrogen groups includes alkyl, alkenyl, alkynyl, haloalkyl, aryl, hydroxyl, alkoxyl, cyano, amido, carboxyl, amino, halogen, etc.

An “alkenyl” group refers, in another embodiment, to an unsaturated hydrocarbon, including straight chain, branched chain and cyclic groups having one or more double bond. The alkenyl group may have one double bond, two double bonds, three double bonds etc. Examples of alkenyl groups are ethenyl, propenyl, butenyl, cyclohexenyl etc. The alkenyl group may be unsubstituted or substituted by one or more groups selected from halogen, hydroxy, alkoxy carbonyl, amido, alkylamido, dialkylamido, nitro, amino, alkylamino, dialkylamino, carboxyl, thio to and thioalkyl.

An “alkynyl” group refers, in another embodiment, to an unsaturated hydrocarbon, including straight chain, branched chain and cyclic groups having one or more triple bond. The alkynyl group may have one triple bond, two triple bonds, three triple bonds etc. Examples of alkynyl groups are ethynyl, propynyl, butynyl, etc. The alkynyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

In one embodiment, this invention provides a polymer represented by the structure of formula (25):

-[(A)ₒ-(B)ₚ—(C)_{q}]ᵣ— (25)

wherein A, B and C may be positioned in any order relative to one another such that A, B and C are either uniformly distributed as blocks throughout the polymer or A, B and C are randomly distributed throughout the polymer and wherein A is a monomer unit represented by the structure:

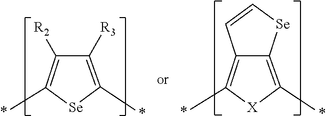

wherein

B is a monomer unit comprised of a monocyclic or bicyclic aryl or heteroaryl group wherein said monocyclic or bicyclic aryl or heteroaryl group is optionally substituted with 1-3 groups comprising CN, COOH, C₁-C₁₈ alkyl, OH, O—(C₁-C₁₈ alkyl), SH, S—(C₁-C₁₈ alkyl), NH₂, NH—(C₁-C₁₈ alkyl), or N(C₁-C₁₈ alkyl)₂;

C is a monomer unit comprised of a substituted or unsubstituted vinyl or acetylene group;

o is an integer from 1-10,000;

p is an integer from 0-10,000;

q is an integer from 0-10,000;

r is an integer from 2-10,000;

R² is H, F, Cl, Br, I, C₁-C₆alkyl, S, O, NH, Y—H or Y—(C₁-C₆ alkyl) wherein if R³ is H then R² is not C₁-C₆alkyl;

R³ is H, F, Cl, Br, I, C₁-C₆ alkyl, Z-H or Z-(C₁-C₆ alkyl) wherein if R² is H then R³ is not C₁-C₆alkyl;

or

R² and R³ combine to form a 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];

or

R² and R³ combine to form Y-ring-Z having the following structure;

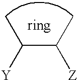

wherein said ring of Y-ring-Z is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];

R⁴ is C₁-C₆ alkyl;

R⁵ is C₁-C₆ alkyl;

Y is O, S, Se, NR⁶ and C(R⁷)(R⁸);

Z is O, S, Se, NR⁹ and C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl; and

R¹¹ is H, CN, C₁-C₆ alkyl, OH, SH, NH₂ or aryl;

X is S, Se.

In another embodiment, the polymer of formula (25), wherein p and q are 0 and X is S or Se.

In one embodiment, the polymer of formula (25) comprises a B monomer unit represented by the structure:

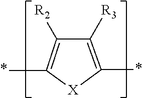

wherein: X is O, Te, S, NH, PH, Si(C₁-C₆ alkyl)₂ or BH;

R² is H, F, Cl, Br, I, C₁-C₆alkyl, Y—H and Y—(C₁-C₆ alkyl);

R³ is H, F, Cl, Br, I, C₁-C₆ alkyl, Z—H and Z—(C₁-C₆ alkyl);

or

R² and R³ combine to form a substituted or unsubstituted 4-8 membered ring comprising 0-3 double bonds and 0-3 heteroatoms selected from O, N, Se or S;

or

R² and R³ combine to form Y-ring-Z having the following structure;

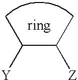

wherein said ring of Y-ring-Z is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];

R⁴ is C₁-C₆ alkyl;

R⁵ is C₁-C₆ alkyl;

Y is O, S, Se, NR⁶ and C(R⁷)(R⁸);

Z is O, S, Se, NR⁹ and C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl; and

R¹¹ is H, CN, C₁-C₆ alkyl, OH, SH, NH₂ or aryl.

In another embodiment, B is a monomer unit represented by the structure:

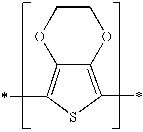

In another embodiment, B is a substituted or unsubstituted thiophene.

In another embodiment, B is a monomer unit represented by the structure:

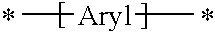

In one embodiment, the polymer of formula (25) comprises a C monomer unit represented by the structure:

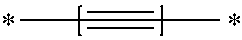

In one embodiment, the polymer of formula (25) comprises a C monomer unit represented by the structure:

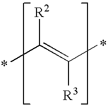

wherein: R² is H, Cl, Br, I, C₁-C₆alkyl, Y—H or Y—(C₁-C₆ alkyl);

R³ is H, Cl, Br, I, C₁-C₆ alkyl, Z-H or Z-(C₁-C₆ alkyl);

Y is O, S, NR⁶ and C(R⁷)(R⁸);

Z is O, S, NR⁹ and C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl; and

R¹¹ is H, CN, C₁-C₆ alkyl, OH, SH, NH₂ or aryl.

In another embodiment, C is a monomer unit represented by the structure:

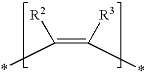

wherein: R² is H, F, Cl, Br, I, C₁-C₆alkyl, Y—H and Y—(C₁-C₆ alkyl);

R³ is H, F, Cl, Br, I, C₁-C₆ alkyl, Z—H and Z—(C₁-C₆ alkyl);

or

R² and R³ combine to form a substituted or unsubstituted 4-8 membered ring containing 0-3 double bonds and 0-3 heteroatoms selected from O, N or S;

or

R² and R³ combine to form Y-ring-Z having the following structure;

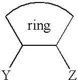

wherein said ring is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];

R⁴ is C₁-C₆ alkyl;

R⁵ is C₁-C₆ alkyl;

Y is O, S, Se, NR⁶ and C(R⁷)(R⁸);

Z is O, S, Se, NR⁹ and C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl; and

R¹¹ is H, CN, C₁-C₆ alkyl, OH, SH, NH₂ or aryl.

In one embodiment, the polymers of this invention comprise A, B and C monomer units wherein A, B and C may be positioned in any order relative to one another. In another embodiment, A, B and C are incorporated into the polymer in the order of A-B-C. In another embodiment, A, B and C are incorporated into the polymer in the order of B-A-C. In another embodiment, A, B and C are incorporated into the polymer in the order of C-B-A. In another embodiment, A, B and C are incorporated into the polymer in the order of A-C-B. In another embodiment, A, B and C are incorporated into the polymer in the order of C-A-B. In another embodiment, A, B and C are incorporated into the polymer in the order of B-C-A. In another embodiment, A, B and C are randomly distributed throughout the polymer. In another embodiment, A, B and C are distributed throughout the polymer in alternating blocks, with such alteration comprising any pattern of any order of A, B, C, repeated at any desired interim, as will be appreciated by one skilled in the art.

In one embodiment, the monomer units of the polymers of this invention are A, B, C, or any combination thereof.

In another embodiment, p of formula (25) is 0. In another embodiment, p is 0 and A and C are randomly distributed throughout the polymer. In another embodiment, p is 0 and A and C are distributed throughout the polymer in alternating blocks. In another embodiment, q is 0. In another embodiment, q is 0 and A and B are randomly distributed throughout the polymer. In another embodiment, q is 0 and A and B are distributed throughout the polymer in alternating blocks. In another embodiment p and q are 0. In another embodiment, p and q are 0 and n is between 2-500. In another embodiment, p and q are 0 and n is 2. In another embodiment, p and q are 0 and n is 4. In another embodiment, p and q are 0 and n is 10.

In one, embodiment, a polymer of this invention is represented by the structure of formula (53):

-(A)ₙ- (53)

wherein A is a monomer unit represented by the structure:

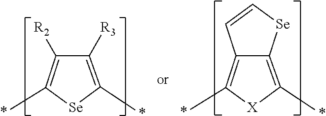

wherein: n is an integer from 2-10,000;

and

R², R³ and X are as defined for the polymer of formula (25).

In another embodiment, the compound of formula (53) wherein X is S or Se.

In another embodiment, the monomer unit A is represented by the structure of formula (26):

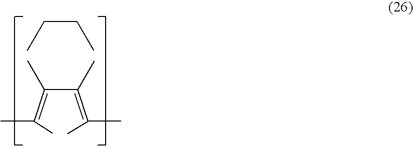

In another embodiment, the monomer unit A is represented by the structure of formula (27):

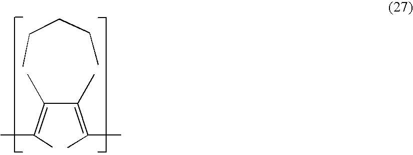

In another embodiment, the monomer unit A is represented by the structure of formula (28):

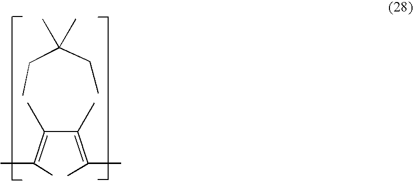

In another embodiment, the monomer unit A is represented by the structure of formula (29):

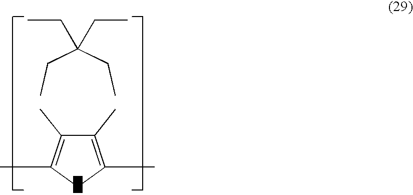

In another embodiment, the monomer unit A is represented by the structure of formula (30):

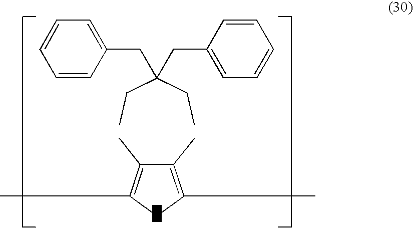

In another embodiment, the monomer unit A is represented by the structure of formula (31):

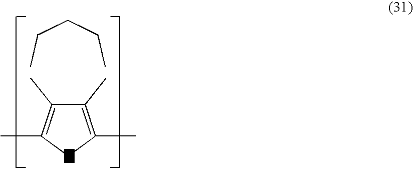

In another embodiment, the monomer unit A is represented by the structure of formula (32):

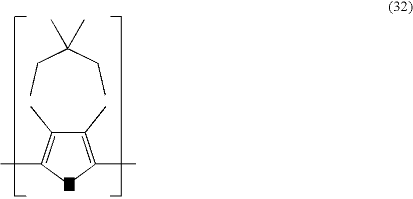

In another embodiment, the monomer unit A is represented by the structure of formula (33):

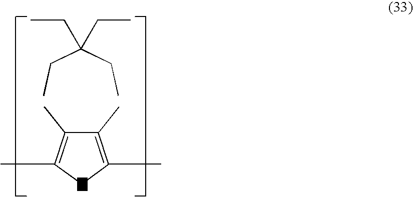

In another embodiment, the monomer unit A is represented by the structure of formula (34):

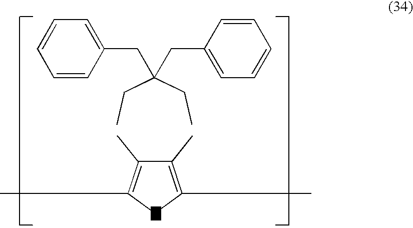

In another embodiment, the monomer unit A is represented by the structure of formula (35):

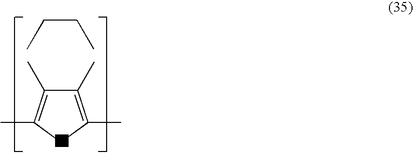

In another embodiment, the monomer unit A is represented by the structure of formula (36):

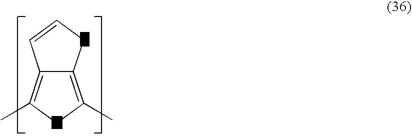

In another embodiment, the monomer unit A is represented by the structure of formula (37):

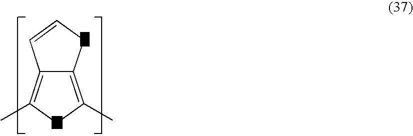

In another embodiment, the monomer unit A is represented by the structure of formula (38):

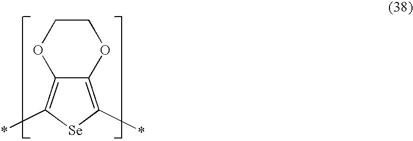

In another embodiment, the monomer unit A is represented by the structure of formula (39):

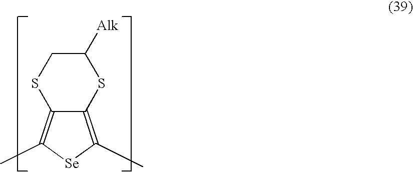

wherein Alk is a C₁-C₁₂ alkyl. In another embodiment, the alkyl is ethyl, butyl, hexyl, octyl or dodecyl.

In another embodiment, the monomer unit A is represented by the structure of formula (40):

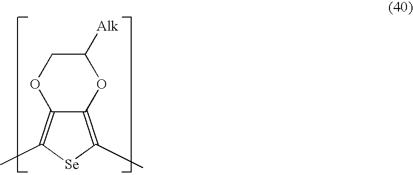

wherein Alk is a C₁-C₁₂ alkyl. In another embodiment, the alkyl is ethyl, butyl, hexyl, octyl or dodecyl.

In one embodiment, the polymers of this invention are conductive polymers. In some embodiments, such polymers will have a conductivity of about 10 S·cm⁻¹. In another embodiment, the polymers of this invention have a conductivity in the range of between about 1-5 S·cm⁻¹. In another embodiment, the polymers of this invention have a conductivity in the range of between about 5-10 S·cm⁻¹. In another embodiment, the polymers of this invention have a conductivity in the range of between about 8-10 S·cm⁻¹. In another embodiment, the polymers of this invention have a conductivity in the range of between about 10-12 S·cm⁻¹. In another embodiment, the polymers of this invention have a conductivity in the range of between about 10-100 S·cm⁻¹. In another embodiment, the polymers of this invention have a conductivity in the range of between about 10⁻²-10 S·cm⁻¹.

In one embodiment, conductivity measurement are carried out via any means known in the art, for example, using a two point probe connected to a FLUKE F115 multimeter and constant-current source system (EG&G PAR 236 Potentiostat/Galvanostat). The electrochemically prepared polymers can be tested using freshly prepared films using 4 probe conductivity instrument.

In one embodiment, the polymers of this invention comprise a dopant. In another embodiment, the dopant is p-type. In another embodiment, the p-type dopant is Br₃⁻, I₃⁻, AsF₆⁻, ClO₄⁻, BF₄⁻ or FeCl₄⁻. In another embodiment, the dopant is n-type. In another embodiment, the n-type dopant is Li⁺, Na⁺ or K⁺.

The term “dopant” refers, in one embodiment to a substance which is added to a polymer in small quantities in order to cause the mixture of polymer and dopant to be electrically conductive. The polymers of the present invention may be termed self-doped, that is, what may be viewed as a dopant is an integral part of the polymer rather than a separate substance added to the polymer to form a mixture of polymer and dopant. However, though these polymers are electrically conductive without a dopant, the magnitude of the conductivity can be increased by adding a dopant material.

In one embodiment, this invention provides a process for preparing a compound represented by the structure of formula (1):

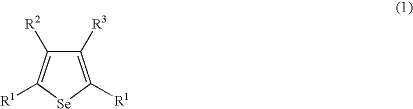

wherein: R¹ is H, F, Cl, Br, I, SH, OSO₂CH₃ or OSO₂CF₃;

R² is H, F, Cl, Br, I, C₁-C₆alkyl, S, O, NH, Y—H or Y—(C₁-C₆ alkyl) wherein if R³ is H then R² is not C₁-C₆alkyl;

R³ is H, F, Cl, Br, I, C₁-C₆ alkyl, Z—H or Z—(C₁-C₆ alkyl) wherein if R² is H then R³ is not C₁-C₆alkyl;

or

R² and R³ combine to form a substituted or unsubstituted 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];

or

R² and R³ combine to form Y-ring-Z having the following structure;

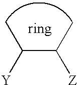

wherein said ring of Y-ring-Z is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];

R⁴ is C₁-C₆ alkyl;

R⁵ is C₁-C₆ alkyl;

Y is O, S, NR⁶ and C(R⁷)(R⁸);

Z is O, S, NR⁹ and C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl;

R¹¹ is H, CN, C₁-C₆ alkyl, OH, SH, NH₂ or aryl;

said process comprising the steps of:

a) reacting 3,4-dimethoxyselenophene with a nucleophile to yield 3,4-substituted-selenophene:

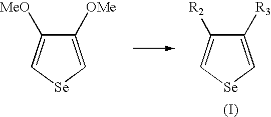

b) optionally brominating 3,4-substituted-selenophene (I) of step (a), obtaining 2,5 dibromoselenophene:

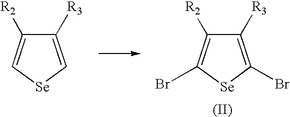

and c) optionally substituting the bromide of compound (II) of step (b) with a nucleophile, obtaining the compound of formula (1).

In one embodiment, the process for preparing a compound of formula (1) and/or a polymer and/or dimer of this invention comprises the step of reacting 3,4-dimethoxyselenophene with a nucleophile to yield 3,4-substituted-selenophene. In another embodiment, 3,4-dimethoxyselenophene is prepared by condensation of 2,3-dimethoxybutadiene with selenium dichloride. In another embodiment, said nucleophile is Y-ring-Z, Y—H, Y—(C₁-C₆ alkyl), ethane-1,2-diol, propane-1,3-diol, ethane-1,2-dithiane, propane-1,2-dithiane, 2-mercapto-ethanol, or 3-mercapto-propanol wherein said nucleophile is optionally substituted or unsubstituted.

In one embodiment, the process for preparing a compound of formula (1), and/or a polymer and/or dimer of this invention comprise the step of brominating 3,4-substituted-selenophene (1) of step (a), obtaining 2,5 dibromo-selenophene. In another embodiment, the bromination step is catalyzed by an acid. In another embodiment, the acid is para-toluenesulfonic acid. In another embodiment, the brominating step comprises a brominating agent. In another embodiment, said brominating agent is bromine. In another embodiment, said brominating agent is N-bromosuccinimide.

In one embodiment, this invention provides a process for preparing a polymer of formula (25):

-[(A)ₒ-(B)ₚ—(C)_{q}]ᵣ— (25)

wherein A, B and C may be positioned in any order relative to one another such that A, B and C are either uniformly distributed as blocks throughout the polymer or A, B and C are randomly distributed throughout the polymer and wherein:

A is a monomer unit represented by the structure:

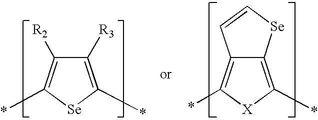

B is a monomer unit comprised of a monocyclic or bicyclic aryl or heteroaryl group wherein said monocyclic or bicyclic aryl or heteroaryl group is optionally substituted with 1-3 groups comprising CN, COOH, C₁-C₁₈ alkyl, OH, O—(C₁-C₁₈ alkyl), SH, S—(C₁-C₁₈ alkyl), NH₂, NH—(C₁-C₁₈ alkyl), or N(C₁-C₁₈ alkyl)₂;

C is a monomer unit comprised of a substituted or unsubstituted vinyl or acetylene group;

o is an integer from 1-10,000;

p is an integer from 0-10,000;

q is an integer from 0-10,000;

r is an integer from 2-10,000;

R² is H, F, Cl, Br, I, C₁-C₆alkyl, S, O, NH, Y—H and Y—(C₁-C₆ alkyl) wherein if R³ is H then R² is not C₁-C₆alkyl;

R³ is H, F, Cl, Br, I, C₁-C₆ alkyl, Z—H and Z—(C₁-C₆ alkyl) wherein if R² is H then R³ is not C₁-C₆alkyl;

or

R² and R³ combine to form a 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];

or

R² and R³ combine to form Y-ring-Z having the following structure;

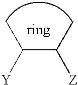

wherein said ring of Y-ring-Z is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];

R⁴ is C₁-C₆ alkyl;

R⁵ is C₁-C₆ alkyl;

Y is O, S, Se, NR⁶ or C(R⁷)(R⁸);

Z is O, S, Se, NR⁹ or C(R¹⁰)(R¹¹);

R⁶ is H, C₁-C₆ alkyl or C(O)(C₁-C₆ alkyl);

R⁷ is H, CN, C₁-C₆ alkyl, OH, SH, NH₂ or aryl;

R⁸ is H, CN, C₁-C₆ alkyl, OH, SH, NH₂ or aryl;

R⁹ is H, C₁-C₆ alkyl or C(O)(C₁-C₆ alkyl);

R¹⁰ is H, CN, C₁-C₆ alkyl, OH, SH, NH₂ or aryl; and

R¹¹ is H, CN, C₁-C₆ alkyl, OH, SH, NH₂ or aryl;

X is S or Se;

said process comprises the step of polymerizing a 2,5-dibromoselenophene or 2,5-diiodoselenophene monomer unit of A with monomer unit B, monomer unit C or any combination thereof, in the presence of Ni(COD)₂ or by heating said monomer units to a temperature range of between 20-150° C.; or said process comprises the step of polymerizing a selenophene monomer unit of A, with monomer unit B, monomer unit C or any combination thereof, in the presence of FeCl₃ or polymerizing said monomers electrochemically, wherein position 2, 5 of said selenophene monomer unit of A are hydrogens.

In one embodiment the process of preparing a polymer of formula (25) comprises the step of polymerizing a 2,5-dibromoselenophene unit or 2,5-dioodoselenophen unit of A with monomer unit B, monomer unit C or any combination thereof, in the presence of Ni(COD)₂.

In one embodiment, the process of preparing a polymer of formula (25) comprises the step of polymerizing a selenophene unit of A with monomer unit B, monomer unit C or any combination thereof, in the presence of FeCl₃.

In one embodiment, the process of preparing a polymer of formula (25) comprises the step of polymerizing a 2,5-dibromoselenophene unit or 2,5-diiodoselenophene unit of A with monomer unit B, monomer unit C or any combination thereof, by heating said monomers to a temperature range of between 20-150° C. or polymerizing said monomers.

In one embodiment, the process of preparing a polymer of formula (25) comprises the step of polymerizing a selenophene unit of A with monomer unit B, monomer unit C or any combination thereof, electrochemically.

In one embodiment, this invention provides a process for preparing a polymer of formula (53):

-(A)ₙ- (53)

wherein: A is a monomer unit represented by the structure:

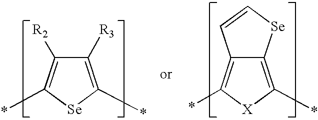

wherein: n is an integer from 2-10,000;

and

R², R³ and X are as defined for the polymer of formula (25).

said process comprises the step of polymerizing a 2,5-dibromoselenophene or 2,5-diiodoselenophene monomer unit of A, in the presence of Ni(COD)₂ or by heating said monomer units to a temperature range of between 20-150° C.; or said process comprises the step of polymerizing a selenophene monomer unit of A, in the presence of FeCl₃ or polymerizing said monomers electrochemically, wherein position 2, 5 of said selenophene monomer unit of A are hydrogens.

In one embodiment the process of preparing a polymer of formula (53) comprises the step of polymerizing a 2,5-dibromoselenophene unit or 2,5-diiodoselenophene unit of A with monomer unit B, monomer unit C or any combination thereof, in the presence of Ni(COD)₂.

In one embodiment, the process of preparing a polymer of formula (53) comprises the step of polymerizing a selenophene unit of A with monomer unit B, monomer unit C or any combination thereof, in the presence of FeCl₃.

In one embodiment, the process of preparing a polymer of formula (53) comprises the step of polymerizing a 2,5-dibromoselenophene unit or 2,5-diiodoselenophene unit of A with monomer unit B, monomer unit C or any combination thereof, by heating said monomers to a temperature range of between 20-150° C. or polymerizing said monomers.

In one embodiment, the process of preparing a polymer of formula (53) comprises the step of polymerizing a selenophene unit of A with monomer unit B, monomer unit C or any combination thereof, electrochemically.

In one embodiment, this invention provides a process for preparing a dimer represented by formula 54:

-(A)₂- (54)

wherein: A is a monomer unit represented by the structure:

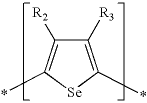

and

R², R³ and X are as defined for formula (25);

said process comprising the steps of:

a) reacting 3,4-dimethoxyselenophene with a nucleophile to yield 3,4-substituted-selenophene:

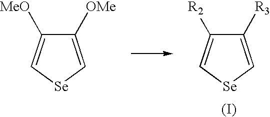

b) optionally brominating 3,4-substituted-selenophene (I) of step (a), obtaining 2,5 dibromo-selenophene:

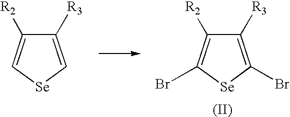

and c) treating the selenophene of compound (I) of step (a) or the bromide of compound (II) of step (b) with n-butyllithium and CuCl₂:

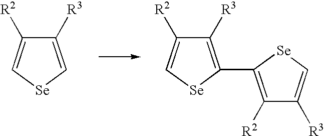

In one embodiment, the process for the preparation of a dimer of formula (54) comprises the step of adding n-butyllithium and CuCl₂. In another embodiment, treatment of the selenophene of compound (I) of step (a) or the bromide of compound (II) of step (b) with n-butyllithium and CuCl₂ starts at −78° C. and warms to room temperature.

In one embodiment, this invention provides a process for the preparation of a polymer of formula (53) comprising monomer A of formula (38)

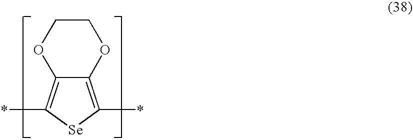

said process comprises polymerizing a compound of formula (3):

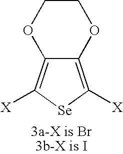

wherein said polymerization comprises heating said compound (3) to a temperature range of between 20-150° C. or reacting said compound (3) with Ni(COD)₂ catalyst.

In one embodiment, this invention provides a process for the preparation of a polymer of formula (53) comprising monomer A of formula (38)

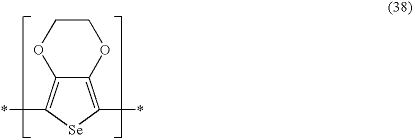

said process comprises polymerizing a compound of formula (40):

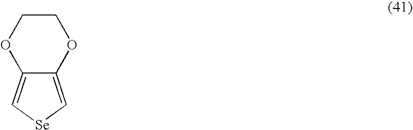

wherein said polymerization comprises adding FeCl₃ to compound (41) or polymerizing compound (41) by electrochemical means.

In one embodiment, this invention provides a process for the preparation of a polymer of formula (53) comprising monomer A of formula (31)

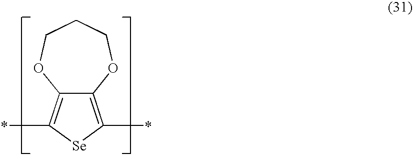

said process comprises polymerizing a compound of formula (4):

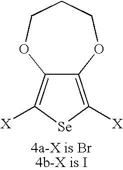

wherein said polymerization comprises heating said compound (4) to a temperature range of between 20-150° C. or reacting said compound with Ni(COD)₂ catalyst.

In one embodiment, this invention provides a process for the preparation of a polymer of formula (53) comprising monomer A of formula (31)

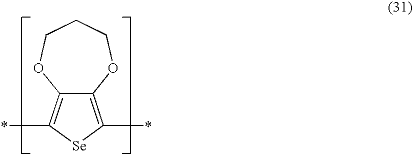

said process comprises polymerizing a compound of formula (8):

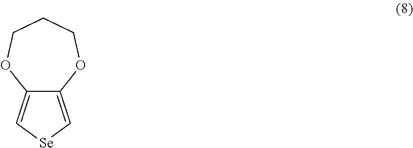

wherein said polymerization comprises adding FeCl₃ to compound (8) or polymerizing compound (8) by electrochemical means,

In one embodiment, this invention provides a process for the preparation of a polymer of formula (53) comprising monomer A of formula (32):

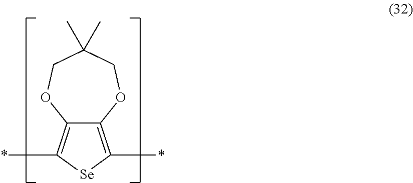

said process comprises polymerizing a compound of formula (5):

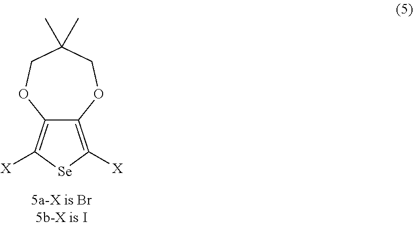

wherein said polymerization comprises heating said compound (5) to a temperature range of between 20-150° C. or reacting said compound (5) with Ni(COD)₂ catalyst.

In one embodiment, this invention provides a process for the preparation of a polymer of formula (53) comprising monomer A of formula (32):

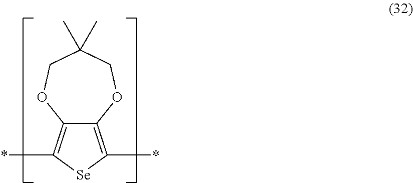

said process comprises polymerizing a compound of formula (9):

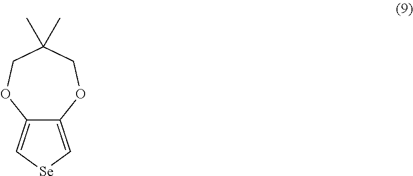

wherein said polymerization comprises adding FeCl₃ to compound (9) or polymerizing compound (9) by electrochemical means.

In one embodiment, this invention provides a process for the preparation of a polymer of formula (53) comprising monomer A of formula (33)

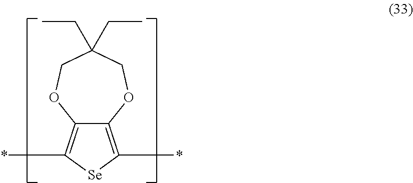

said process comprises polymerizing a compound of formula (6):

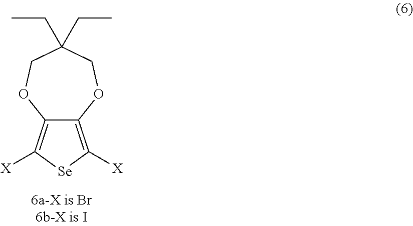

wherein said polymerization comprises heating said compound (6) to a temperature range of between 20-150° C. or reacting said compound (6) with Ni(COD)₂ catalyst.

In one embodiment, this invention provides a process for the preparation of a polymer of formula (53) comprising monomer A of formula (33)

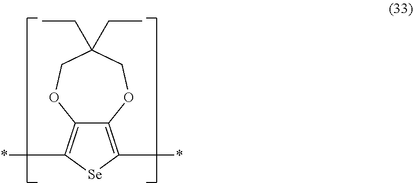

said process comprises polymerizing a compound of formula (10):

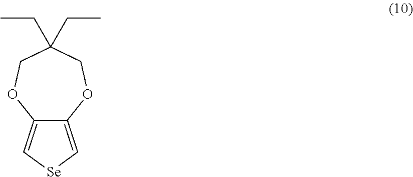

wherein said polymerization comprises adding FeCl₃ to compound (10) or polymerizing compound (10) by electrochemical means.

In one embodiment, this invention provides a process for the preparation of a polymer of formula (53) comprising monomer A of formula (34)

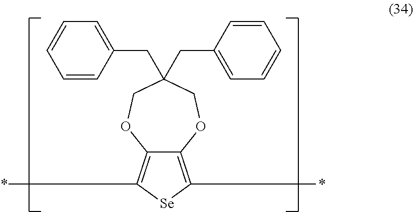

said process comprises polymerizing a compound of formula (7):

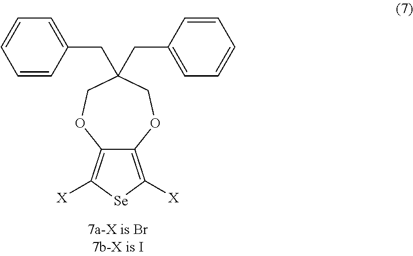

wherein said polymerization comprises heating said compound (7) to a temperature range of between 20-150° C. or reacting said compound (7) with Ni(COD)₂ catalyst.

In one embodiment, this invention provides a process for the preparation of a polymer of formula (53) comprising monomer A of formula (34)

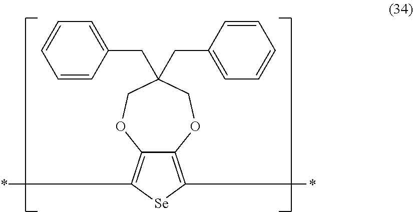

said process comprises polymerizing a compound of formula (11):

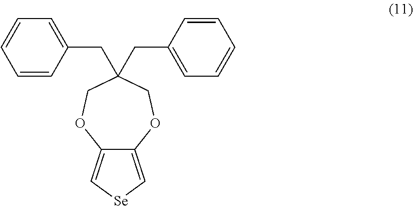

wherein said polymerization comprises adding FeCl₃ to compound (11) or polymerizing compound (11) by electrochemical means.

In one embodiment, this invention provides a process for the preparation of a polymer of formula (53) comprising monomer A of formula (39) or (40):

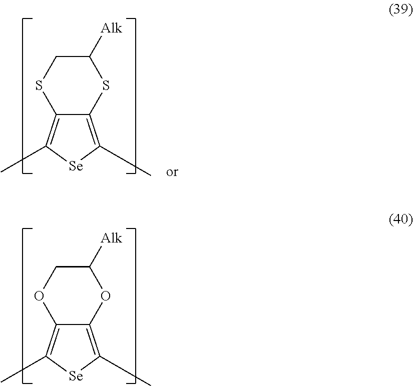

wherein Alk is a C₁-C₁₂ alkyl;

said process comprises polymerizing a compound of formula (43) or (12f) respectively:

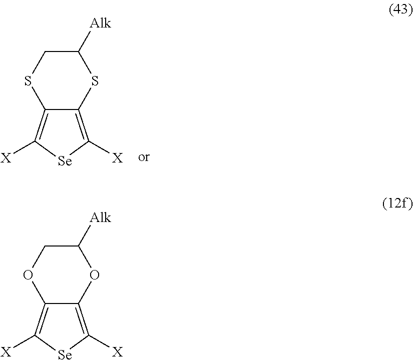

wherein Alk is C₁-C₁₂ alkyl and X is Br or I.
wherein said polymerization comprises heating said compound (43) or (12f) to a temperature range of between 20-150° C. or reacting said compound (43) or (10f) with Ni(COD)₂ catalyst.

In one embodiment, this invention provides a process for the preparation of a polymer of formula (53) comprising monomer A of formula (39) or (40):

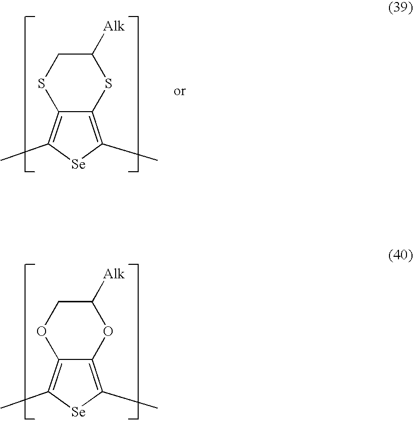

wherein Alk is a C₁-C₁₂ alkyl;

said process comprises polymerizing a compound of formula (43) or (12) respectively:

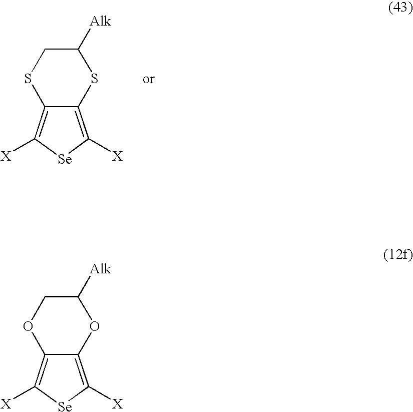

wherein Alk is C₁-C₁₂ alkyl and X is H.
wherein said polymerization comprises adding FeCl₃ to said compound (43) or (12f) or polymerizing said compound (43) or (12f) by electrochemical means. In another embodiment, the alkyl of formula (39) or (40) is ethyl, butyl, hexyl, octyl or dodecyl.

In one embodiment, this invention provides a process for the preparation of a polymer of formula (53) comprising monomer A of formula (26):

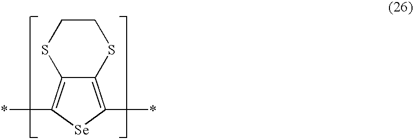

said process comprises polymerizing a compound of formula (13)

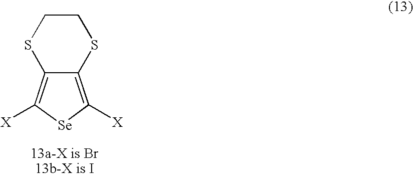

wherein said polymerization comprises heating said compound (13) to a temperature range of between 20-150° C. or reacting said compound (13) with Ni(COD)₂ catalyst.

In one embodiment, this invention provides a process for the preparation of a polymer of formula (53) comprising monomer A of formula (26):

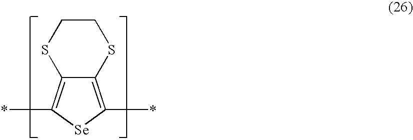

said process comprises polymerizing a compound of formula (18):

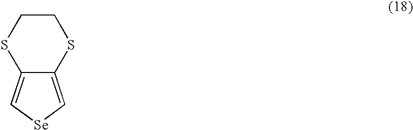

wherein said polymerization comprises adding FeCl₃ to said compound (18) or polymerizing said compound (18) by electrochemical means.

In one embodiment, this invention provides a process for the preparation of a polymer of formula (53) comprising monomer A of formula (27):

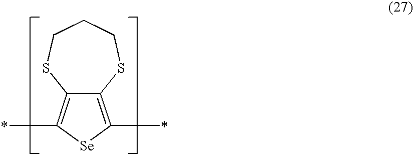

said process comprises polymerizing a compound of formula (14):

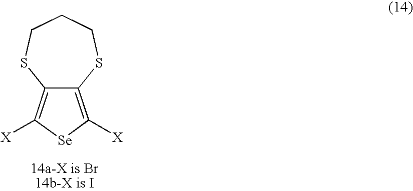

wherein said polymerization comprises heating said compound (14) to a temperature range of between 20-150° C. or reacting said compound (14) with Ni(COD)₂ catalyst.

In one embodiment, this invention provides a process for the preparation of a polymer of formula (53) comprising monomer A of formula (27):

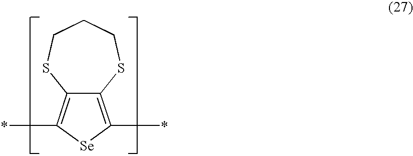

said process comprises polymerizing a compound of formula (19):

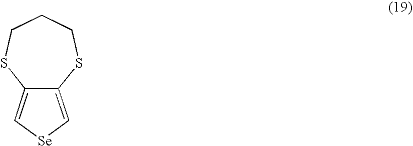

wherein said polymerization comprises adding FeCl₃ to said compound (19) or polymerizing said compound (19) by electrochemical means.

In one embodiment, this invention provides a process for the preparation of a polymer of formula (53) comprising monomer A of formula (28):

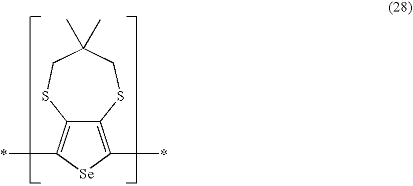

said process comprises polymerizing a compound of formula (15):

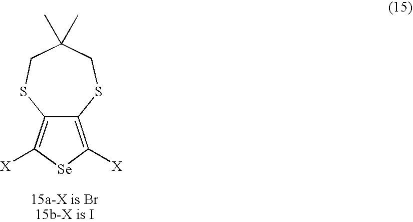

wherein said polymerization comprises heating said compound (15) to a temperature range of between 20-150° C. or reacting said compound (15) with Ni(COD)₂ catalyst.

In one embodiment, this invention provides a process for the preparation of a polymer of formula (53) comprising monomer A of formula (28):

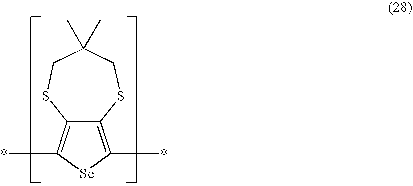

said process comprises polymerizing a compound of formula (20):

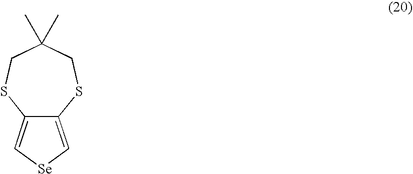

wherein said polymerization comprises adding FeCl₃ to said compound (20) or polymerizing said compound (20) by electrochemical means.

In one embodiment, this invention provides a process for the preparation of a polymer of formula (53) comprising monomer A of formula (29):

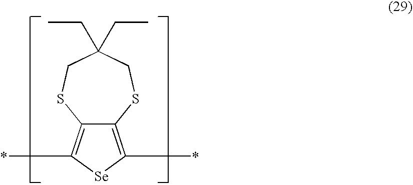

said process comprises polymerizing a compound of formula (16):

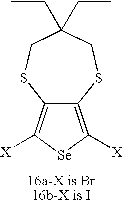

wherein said polymerization comprises heating said compound (16) to a temperature range of between 20-150° C. or reacting said compound (16) with Ni(COD)₂ catalyst.

In one embodiment, this invention provides a process for the preparation of a polymer of formula (53) comprising monomer A of formula (29):

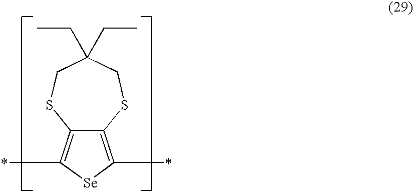

said process comprises polymerizing a compound of formula (21):

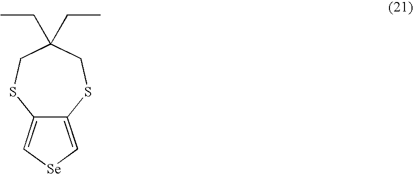

wherein said polymerization comprises adding FeCl₃ to said compound (21) or polymerizing said compound (21) by electrochemical means.

In one embodiment, this invention provides a process for the preparation of a polymer of formula (53) comprising monomer A of formula (30):

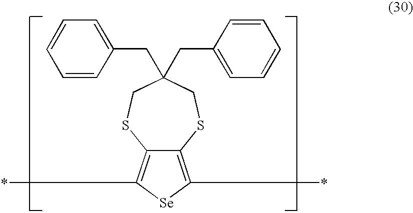

said process comprises polymerizing a compound of formula (17):

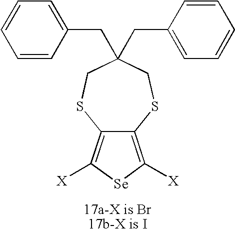

wherein said polymerization comprises heating said compound (17) to a temperature range of between 20-150° C. or reacting said compound (17) with Ni(COD)₂ catalyst.

In one embodiment, this invention provides a process for the preparation of a polymer of formula (53) comprising monomer A of formula (30):

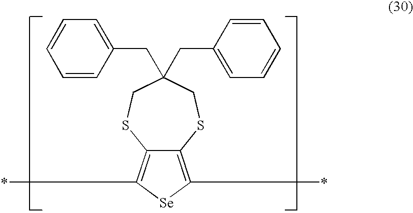

said process comprises polymerizing a compound of formula (22):

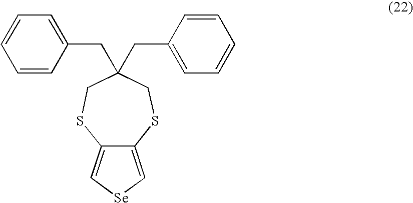

wherein said polymerization comprises adding FeCl₃ to said compound (22) or polymerizing said compound (22) by electrochemical means.

In one embodiment, this invention provides a process for the preparation of a polymer of formula (53) comprising monomer A of formula (36):

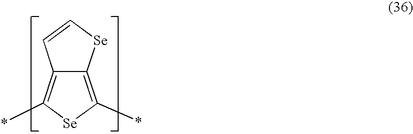

said process comprises polymerizing a compound of (43):

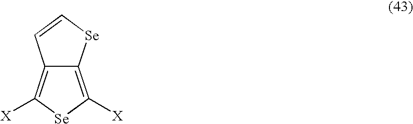

wherein X is Br or I; wherein said polymerization comprises the step of heating said compound (43) to a temperature range of between 20-150° C. or said reacting said compound (43) with Ni(COD)₂ catalyst.

In one embodiment, this invention provides a process for the preparation of a polymer of formula (53) comprising monomer A of formula (36):

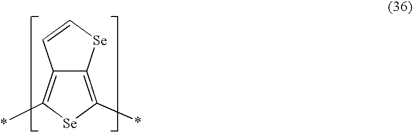

said process comprises polymerizing a compound of (43):

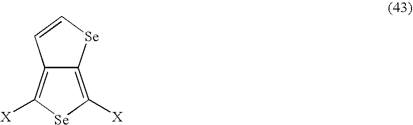

wherein X is H; wherein said polymerization comprises adding FeCl₃ to said compound (43) or polymerizing said compound (43) by electrochemical means.

In one embodiment, this invention provides a process for the preparation of a polymer of formula (53) comprising monomer A of formula (37):

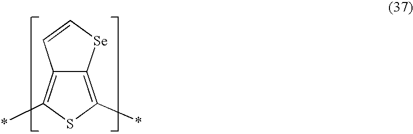

said process comprises polymerizing a compound of (44):

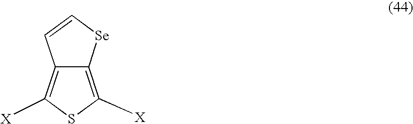

wherein X is Br or I; wherein said polymerization comprises the step of heating said compound (44) to a temperature range of between 20-150° C. or reacting said compound (44) with Ni(COD)₂ catalyst.

In one embodiment, this invention provides a process for the preparation of a polymer of formula (53) comprising monomer A of formula (37):

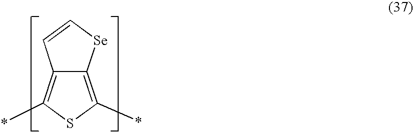

said process comprises polymerizing a compound of (44):

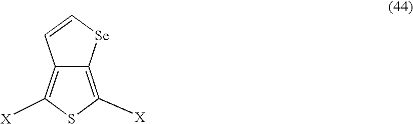

wherein X is H; wherein said polymerization comprises the step of adding FeCl₃ to said compound (44) or polymerizing said compound (44) by electrochemical means.

In another embodiment, the polymers of this invention are prepared by polymerizing a 2,5-dibromoselenophene unit of formula A, optionally with monomer units of formula B, formula C or any combination thereof, by heating said monomer or monomers to a temperature range of between 20-150° C. In another embodiment, the polymers of this invention are prepared by polymerizing a 2,5-dibromoselenophene unit, optionally with monomer units of formula B, formula C or any combination thereof, by heating said monomer or monomers to a temperature range of between 20-80° C. In another embodiment, the polymer is prepared by polymerizing a 2,5-dibromoselenophene unit of formula A, optionally with monomer units of formula B, formula C or any combination thereof, by heating said monomer or monomers to a temperature range of between 20-60° C. In another embodiment, the polymer is prepared by polymerizing a 2,5-dibromoselenophene unit of formula A, optionally with monomer units of formula B, formula C or any combination thereof, by heating said monomer or monomers to a temperature range of between 50-80° C. In another embodiment, the polymer is prepared by polymerizing 2,5-dibromoselenophene unit of formula A, optionally with monomer units of formula B, formula C or any combination thereof, by heating said monomer or monomers to a temperature range of between 50-150° C. In another embodiment, the polymer is prepared by polymerizing 2,5-dibromoselenophene unit of formula A, optionally with monomer units of formula B, formula C or any combination thereof, by heating said monomer or monomers to a temperature range of between 40-80° C. In another embodiment, the polymer is prepared by polymerizing a 2,5-dibromoselenophene unit of formula A, optionally with monomer units of formula B, formula C or any combination thereof, by heating said monomer or monomers to a temperature range of between 60-80° C. In another embodiment, the polymer is prepared by polymerizing 2,5-dibromoselenophene unit of formula A, optionally with monomer units of formula B, formula C or any combination thereof, by heating said monomer or monomers to a temperature range of between 30-50° C. In another embodiment, the polymer is prepared by polymerizing 2,5-dibromoselenophene unit of formula A, optionally with monomer units of formula B, formula C or any combination thereof, by heating said monomer or monomers to a temperature range of between 100-150° C.

In another embodiment, the polymers of this invention are prepared by polymerizing a 2,5-diiodoselenophene unit of formula A, optionally with monomer units of formula B, formula C or any combination thereof, by heating said monomer or monomers to a temperature range of between 20-150° C. In another embodiment, the polymers of this invention are prepared by polymerizing a 2,5-diiodoselenophene unit, optionally with monomer units of formula B, formula C or any combination thereof, by heating said monomer or monomers to a temperature range of between 50-150° C. In another embodiment, the polymer is prepared by polymerizing a 2,5-diiodoselenophene unit of formula A, optionally with monomer units of formula B, formula C or any combination thereof, by heating said monomer or monomers to a temperature range of between 50-80° C. In another embodiment, the polymer is prepared by polymerizing a 2,5-diiodoselenophene unit of formula A, optionally with monomer units of formula B, formula C or any combination thereof, by heating said monomer or monomers to a temperature range of between 70-90° C. In another embodiment, the polymer is prepared by polymerizing 2,5-diiodoselenophene unit of formula A, optionally with monomer units of formula B, formula C or any combination thereof, by heating said monomer or monomers to a temperature range of between 90-120° C. In another embodiment, the polymer is prepared by polymerizing 2,5-diiodoselenophene unit of formula A, optionally with monomer units of formula B, formula C or any combination thereof, by heating said monomer or monomers to a temperature range of between 100-150° C. In another embodiment, the polymer is prepared by polymerizing a 2,5-diiodoselenophene unit of formula A, optionally with monomer units of formula B, formula C or any combination thereof, by heating said monomer or monomers to a temperature range of between 20-80° C. In another embodiment, the polymer is prepared by polymerizing 2,5-diiodoselenophene unit of formula A, optionally with monomer units of formula B, formula C or any combination thereof, by heating said monomer or monomers to a temperature range of between 20-60° C. In another embodiment, the polymer is prepared by polymerizing 2,5-dibromoselenophene unit of formula A, optionally with monomer units of formula B, formula C or any combination thereof, by heating said monomer or monomers to a temperature range of between 50-100° C.

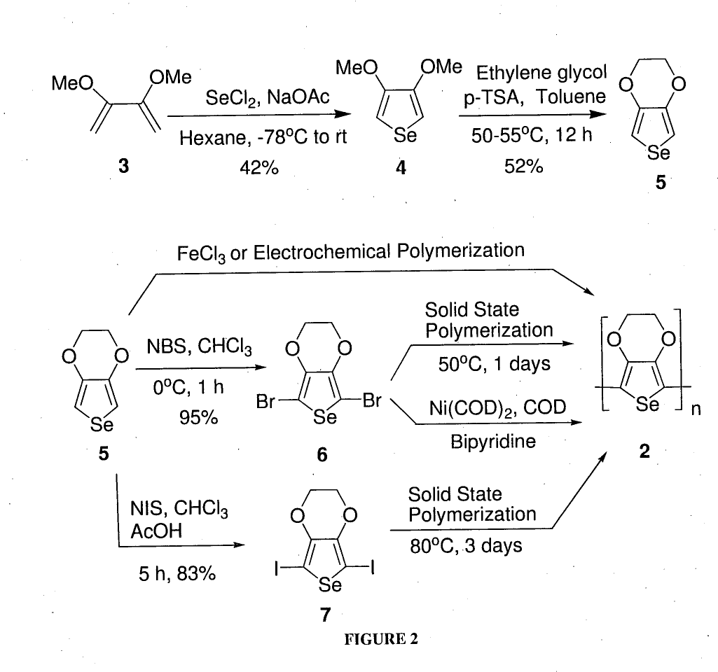

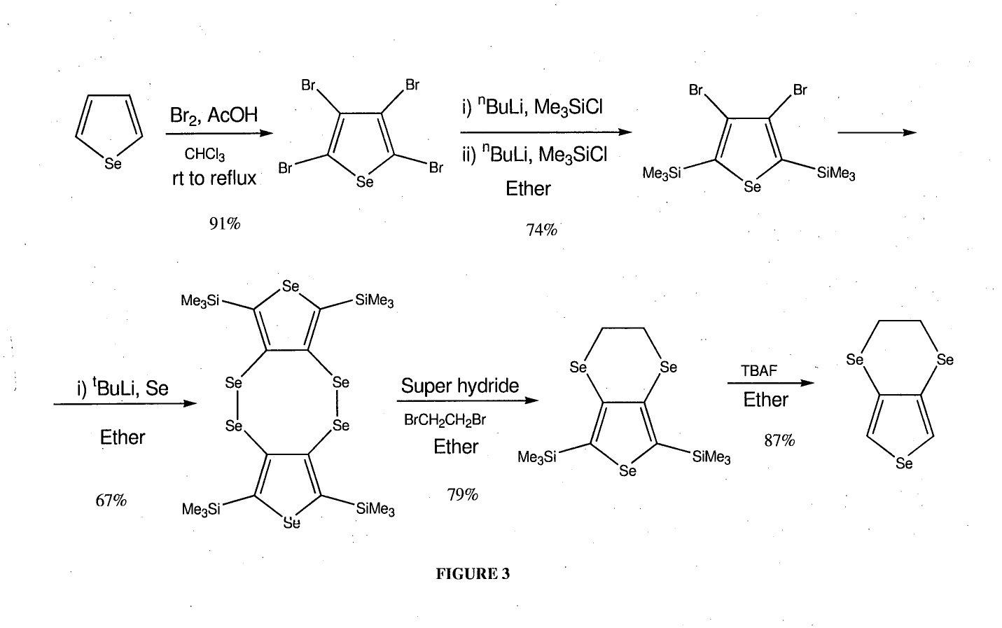

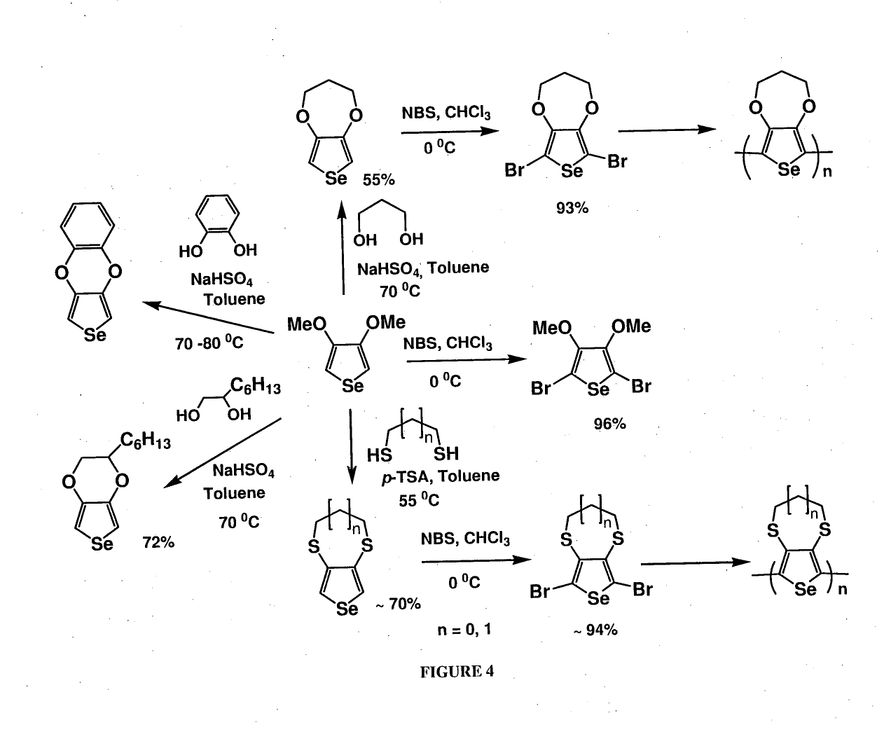

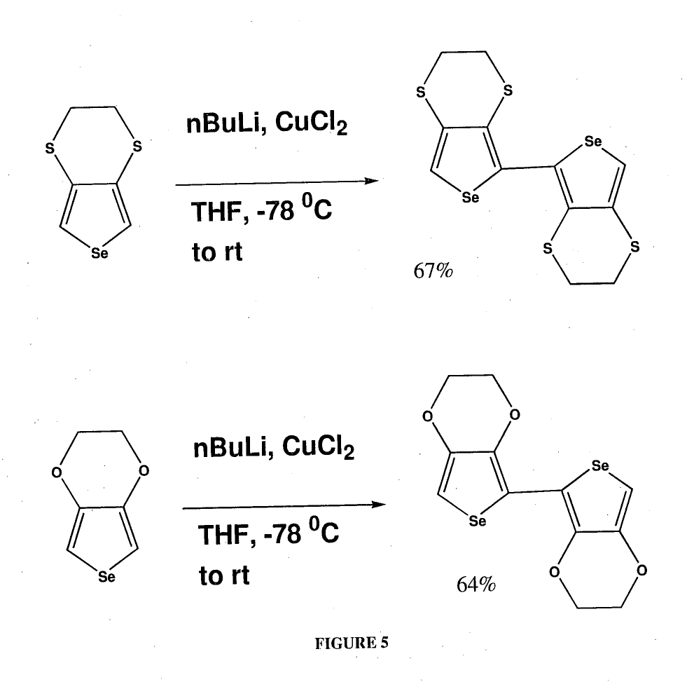

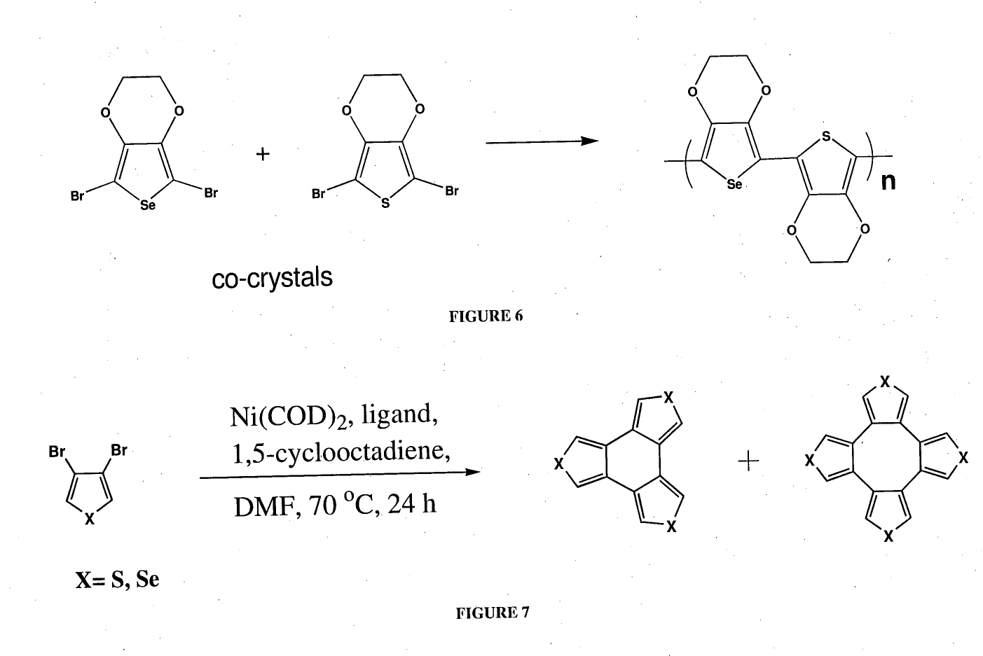

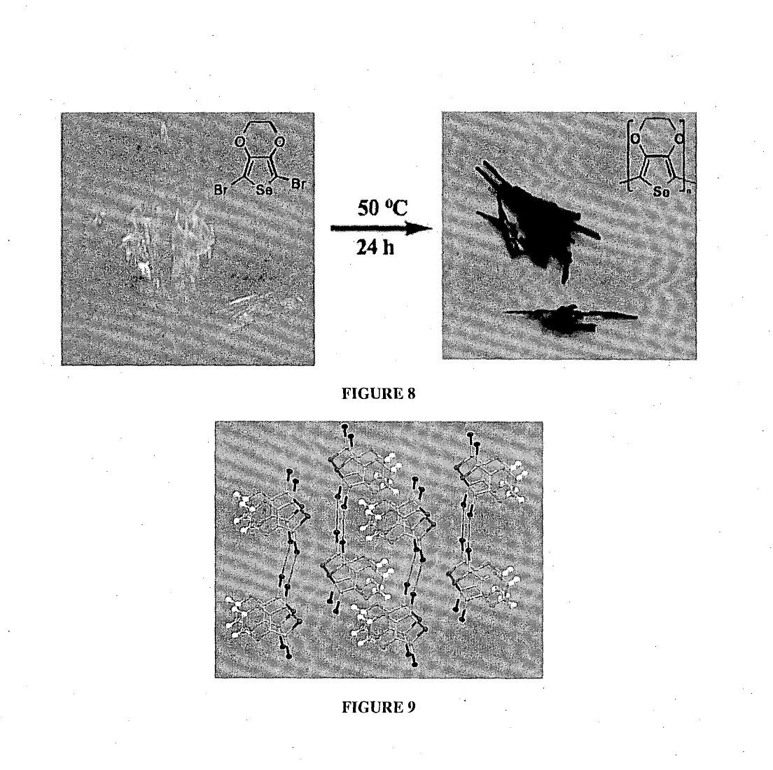

In another embodiment, the process for preparing a polymer of formula (53) wherein A is represented by formula (38) is prepared according to Example 5 and FIG. 2.

In one embodiment, the polymers of this invention are prepared by polymerizing a 2,5-dibromoselenophene or 2,5-diiodoselenophene unit of A, with monomer unit B, monomer unit C or any combination thereof in the presence of Ni(COD)₂ (Nickel-cyclooctadiene complex). In another embodiment Ni(COD)₂ is added in a catalytic amounts. In another embodiment, a process for preparing a polymer of formula (53) comprising monomer A of formula (38) is performed according to Example 6 and FIG. 2.

In one embodiment, the compounds of this invention and/or a polymers and/or dimers of this invention may be prepared by any process as will be known to one skilled in the art, without limitation.

In one embodiment, the polymers of this invention are prepared by polymerizing a selenophene unit of A, monomer unit B, monomer unit C or any combination thereof in the presence of FeCl₃. In another embodiment, the reaction is performed in the presence of chloroform. In another embodiment, a process for preparing a polymer of formula (53) comprising monomer A of formula (38) is performed according to Example 4 and FIG. 2.

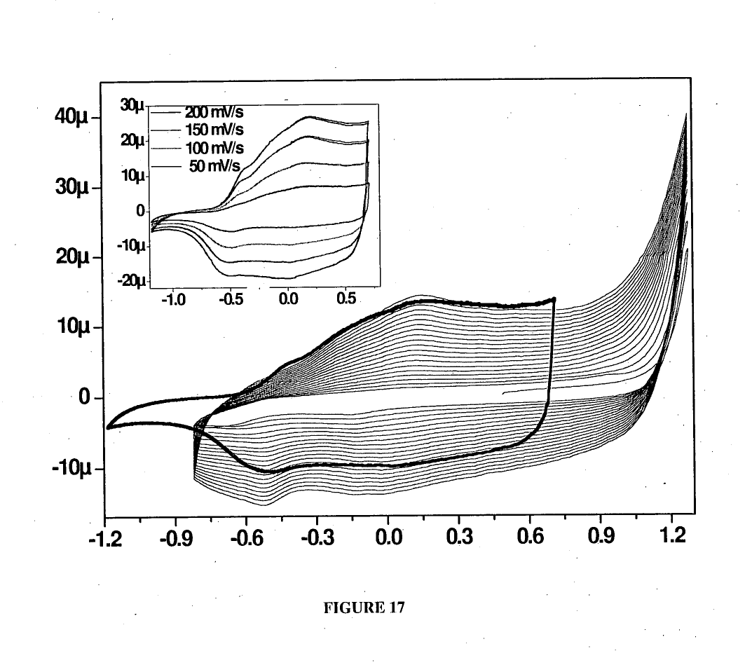

In one embodiment, the polymers of this invention are prepared by polymerizing a selenophene unit of A, monomer unit B, monomer unit C or any combination thereof electrochemically. In another embodiment, the reaction is performed by applying a potential between 1.3 to −1 V versus Ferrocene/Ferrocenium (Fc/Fc⁺). In another embodiment, the reaction is performed by applying a potential between 0.7 to −0.9 V versus Fc/Fc⁺. In another embodiment, the potential is applied in a 200 mV/sec speed. In another embodiment, the potential is applied in a 150 mV/sec speed. In another embodiment, the potential is applied in a 100 mV/sec speed. In another embodiment, the potential is applied in a 50 mV/sec speed. In another embodiment, the electrochemical means is performed according to FIG. 17.

In another embodiment, a polymer of this invention is prepared by brominating or iodizing the monomer followed by heating between 20-90° C. (solid state polymerization). In another embodiment, the conductivity of a polymer prepared by heating provides conductivity in the range of 5-50 S·cm⁻¹.

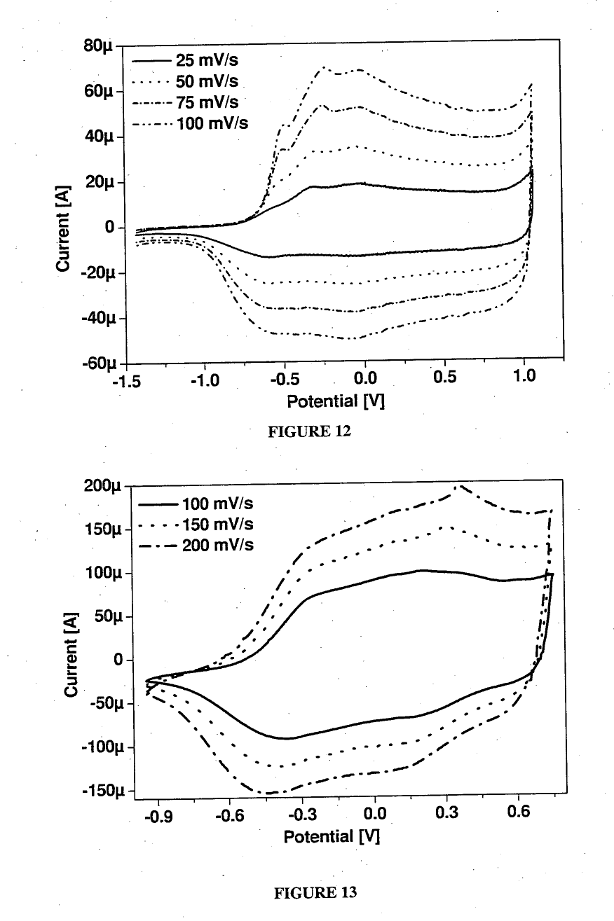

In another embodiment, a polymer of this invention is electropolymerized. In another embodiment, the conductivity of a polymer that was prepared electrochemically shows high conductivity in the range of 5-50 S·cm⁻¹. In another embodiment, under repeated cyclic voltametry (CV) cycles of a monomer of this invention at its oxidation potential, a polymer is produced. In another embodiment, a polymer comprising a monomer of formula (38) (3,4-ethylenedioxyselenophene) was prepared by repeated CV cycles at 1.2V (as presented in FIGS. 11 and 12) to produce insoluble neutral deep-blue or oxidized sky-blue poly(3,4-ethylnedioxyselenophenes) (PEDOS) on the surface of the working electrode.

In one embodiment, the process for preparing a polymer of this invention comprises the step of adding a dopant to the monomer units of this invention. In another embodiment, the dopant is p-type. In another embodiment a p-type dopant comprises Br₃⁻, I₃⁻, AsF₆⁻, ClO₄⁻, BF₄⁻ or FeCl₄⁻. In another embodiment, the dopant is a n-type dopant. In another embodiment a n-type dopant comprises Li⁺, Na⁺ or K⁺.

In one embodiment, conductive polymeric films having holes (p-doped) can be formed via conventional p-dopants which include halogen atoms, e.g., I₂, Cl₂, Br₂, ICl, ICl₃, IBr and IF, Lewis acids, e.g., PF₅, AsF₅, SbF₅, BF₃, BCl₃, SbCl₅, BBr₃ and SO₃, protonic acids, organic acids, or amino acids, e.g., HF, HCl, HNO₃, H₂SO₄, HClO₄, FSO₃H and ClSO₃H, transition metal compounds, e.g., FeCl₃, Fe(OCl)₃, Fe(ClO₄)₃, Fe(CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnX₃ wherein Ln is a lanthanoid and X is an anion, e.g., Cl⁻, Br⁻, I⁻, I₃⁻, HSO₄⁻, SO₄²⁻, NO₃⁻, ClO₄⁻, BF₄⁻, B₁₂F₁₂²⁻, PF₆⁻, AsF₆⁻, FeCl₄⁻, Fe(CN)₆³⁻, and anions of various sulfonic acids, such as aryl-SO₃⁻. Also, O₂, as well as O₃, may be used. Conductive polymeric films employing electrons as carriers as in n-doped polymeric films utilize conventional n-dopants which include the alkali metals (e.g., Li, Na, K, Rb, and Cs), alkaline-earth metals e.g., Ca, Sr, and Ba.

In one embodiment, the polymers of this invention may be doped with conventional p- and n-type dopants post polymerization of the respective monomers. The doping process typically involves treatment of the film material with an oxidizing or reducing agent in a redox reaction to form delocalized ionic centers in the material, with the corresponding counterions derived from the applied dopants. Doping methods comprise for example exposure to a doping vapor in the atmospheric or at a reduced pressure, electrochemical doping in a solution containing a dopant, bringing the dopant in contact with the polymer to be thermally diffused, and ion-implantantion of the dopant into the semiconductor material.

In one embodiment, this invention provides a compound, polymer and/or dimer prepared according to any process as herein described.

In one embodiment, the polymers of this invention that were prepared according to the processes of this invention have a conductivity of 10 S·cm⁻¹. In another embodiment, the polymers of this invention that were prepared according to the processes of this invention have a conductivity in the range of between about 1-5 S·cm⁻¹. In another embodiment, the polymers of this invention that were prepared according to the processes of this invention have a conductivity in the range of between about 5-10 S·cm⁻¹. In another embodiment, the polymers of this invention that were prepared according to the processes of this invention have a conductivity in the range of between about 8-10 S·cm⁻¹. In another embodiment, the polymers of this invention that were prepared according to the processes of this invention have a conductivity in the range of between about 10-12 S·cm⁻¹. In another embodiment, the polymers of this invention that were prepared according to the processes of this invention have a conductivity in the range of between about 10-100 S·cm⁻¹. In another embodiment, the polymers of this invention that were prepared according to the processes of this invention have a conductivity in the range of between about 10⁻²-1 S·cm⁻¹.

In one embodiment, this invention provides a radialene compound represented by the structure of formula (45):

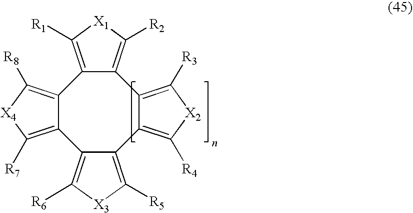

wherein: R¹, R², R³, R⁴, R⁵, R⁶, R⁷ or R⁸ are independently H, F, Cl, Br, I, CN, OH, SH, NH₂, O—(C₁-C₆ alkyl), S—(C₁-C₆ alkyl), NH(C₁-C₆ alkyl), N(R⁹)(R¹⁰), NHC(O)(C₁-C₆ alkyl) and N[(C₁-C₆ alkyl)][C(O)(C₁-C₆ alkyl)] or C(O)OR¹¹;

R⁹ is C₁-C₆ alkyl;

R¹⁰ is C₁-C₆alkyl;

R¹¹ is C₁-C₆ alkyl;

X¹, X², X³ or X⁴ are independently O, S, Se, Te, NH or PH;

and

n is an integer from 0-2.

In one embodiment, R¹, R², R³, R⁴, R⁵, R⁶, R⁷ and R⁸ of compound 45 are H, X¹, X², X³ and X⁴ are independently Se; and n is an integer from 0-1. In another embodiment, X¹, X², X³ and X⁴ is Se; and n is 0. In another embodiment, X¹, X², X³ and X⁴ is Se; and n is 1.

In one embodiment, this invention provides a radialene compound represented by the structure of formula (46):

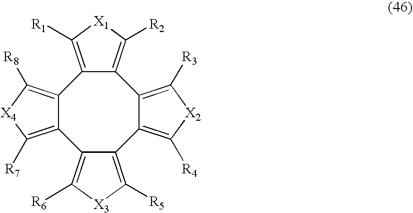

wherein: R¹, R², R³, R⁴, R⁵, R⁶, R⁷ or R⁸ are independently H, F, Cl, Br, I, CN, OH, SH, NH₂, O—(C₁-C₆ alkyl), S—(C₁-C₆ alkyl), NH(C₁-C₆ alkyl), N(R⁹)(R¹⁰), NHC(O)(C₁-C₆ alkyl) and N[(C₁-C₆ alkyl)][C(O)(C₁-C₆ alkyl)] or C(O)OR¹¹;

R⁹ is C₁-C₆ alkyl;

R¹⁰ is C₁-C₆ alkyl;

R¹¹ is C₁-C₆ alkyl; and

X¹, X², X³ or X⁴ are independently O, S, Se, Te, NH or PH;

wherein X¹, X², X³ and X⁴ are not S.

In one embodiment, R¹, R², R³, R⁴, R⁵, R⁶, R⁷ and R⁸ of compound 46 are H, X¹, X², X³ and X⁴ are independently Se; and n is an integer from 0-1. In another embodiment, X¹, X², X³ and X⁴ is Se; and n is 0. In another embodiment, X¹, X², X³ and X⁴ is Se; and n is 1.

In one embodiment, this invention provides a radialene compound represented by the structure of formula (47):

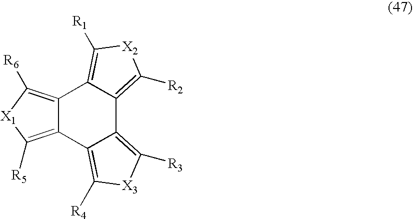

wherein: R¹, R², R³, R⁴, R⁵ or R⁶ are independently H, F, Cl, Br, I, CN, OH, SH, NH₂, O—(C₁-C₆ alkyl), S—(C₁-C₆ alkyl), NH(C₁-C₆ alkyl), N(R⁷)(R⁸), NHC(O)(C₁-C₆ alkyl) and N[(C₁-C₆ alkyl)][C(O)(C₁-C₆ alkyl)] or C(O)OR⁹;

R⁷ is C₁-C₆alkyl;

R⁸ is C₁-C₆alkyl;

R⁹ is C₁-C₆ alkyl;

X¹, X² or X³ are independently O, S, Se, Te, NH or PH;

wherein X¹, X² and X³ are not S.

In one embodiment, R¹, R², R³, R⁴, R⁵ and R⁶ of compound 47 is H, X¹, X² and X³ are Se; and n is an integer from 0-1. In another embodiment, X¹, X² and X³ are Se; and n is 0.

In one embodiment, this invention provides a radialene compound represented by the structure of formula (48):

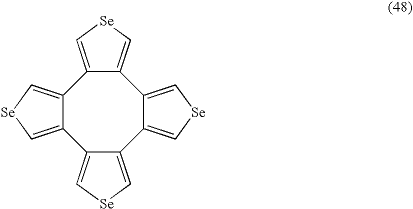

In one embodiment, this invention provides a radialene compound represented by the structure of formula (49):

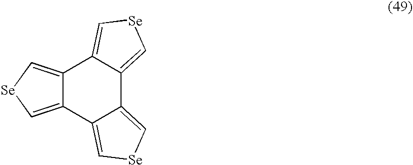

In one embodiment, this invention provides a process for preparing a radialene compound represented by the structure of formula (45):

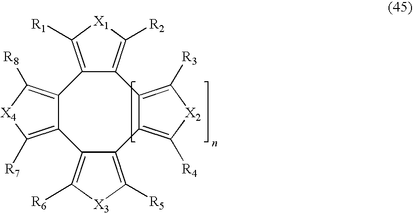

wherein X¹, X², X³, X⁴, R¹, R², R³, R⁴, R⁵, R⁶, R⁷, R⁸ and n are as described above; and wherein, X¹, X², X³ and X⁴ are the same, R¹, R³, R⁵ and R⁷ are the same and R², R⁴, R⁶ and R⁸ are the same; said process comprises reacting a compound represented by formula (50):

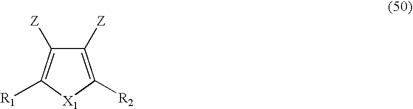

with Ni(COD)₂;

wherein: R¹, R² and X¹ are as defined for formula (61) and

Z is F, Cl, Br, or I.

In one embodiment, R¹ and R² of compound (50) is H, X¹ is S and n is an integer from 0-1. In one embodiment, R¹ and R² of compound (50) is H, X¹ is Se and n is an integer from 0-1. In another embodiment, X¹ of formula (50) is S; and n is 0. In another embodiment, X¹ of formula (50) is S; and n is 1. In another embodiment, X¹ of formula (50) is Se; and n is 0. In another embodiment, X¹ of formula (50) is Se; and n is 1.

In one embodiment, this invention provide a process for the preparation of compound of formula (45), wherein X¹, X², X³ or X⁴ may be different or the same; R¹, R³, R⁵ and R⁷ are different or the same and R², R⁴, R⁶ and R⁸ are different or the same said process comprises reacting compound of formula (50),

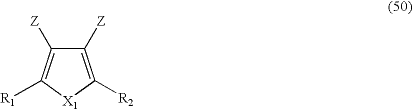

compound of formula (51)

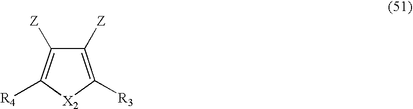

compound of formula (52)

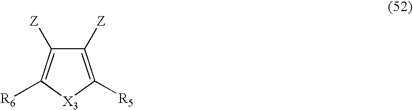

and optionally compound of formula (55)

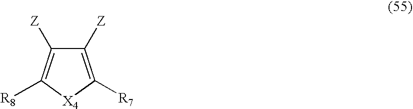

with Ni(COD)₂;

wherein: R¹, R², R³, R⁴, R⁵, R⁶, R⁷, R⁸ and X¹, X², X³ and X¹ are as defined for formula (45) and Z is F, Cl, Br, or I;

In one embodiment, the process of compound of formula (45) comprises reacting a compound of formula (50) with Ni(COD)₂. In one embodiment, the process of compound of formula (45) comprises reacting a compound of formula (50) with Ni(COD)₂ and 1,5-dyclooctadiene. In another embodiment, the process of compound of formula (45) comprises reacting a compound of formula (50) with Ni(COD)₂, 1,5-dyclooctadiene and Ph₃P. In another embodiment, the process of compound of formula (45) comprises reacting a compound of formula (50) with Ni(COD)₂, 1,5-dyclooctadiene and 2,2′-bipyridyl.

In some embodiments, the process for the preparation of compound of formula (45), comprises reacting a compound of formula (50) in the presence of Ni(COD)₂. In one embodiment a mixture of compound of formula (46) and a compound of (47) is obtained.

In another embodiment, the process of preparing the radialene of formula (45) comprises heating a compound of formula (50) with Ni(COD)₂ to a temperature range of between 30-80° C. In another embodiment, the radiallene is prepared by heating compound of formula (50) with Ni(COD)₂ to a temperature range of between 50-70° C. In another embodiment, the radiallene is prepared by heating compound of formula (50) with Ni(COD)₂ to a temperature range of between 40-60° C. In another embodiment, the radiallene is prepared by heating compound of formula (50) with Ni(COD)₂ to a temperature range of between 80-100° C. In another embodiment, the radiallene is prepared by heating compound of formula (50) with Ni(COD)₂ to a temperature range of between 30-70° C.

In one embodiment, this invention is directed to polyselenophene dispersions comprising positively charged polyselenophenes and anions. In another embodiment, this invention is directed to positively charged polyselenophenes-(25) or positively charged polyselenophenes (53) wherein A is a monomer of Formula 26-40 of this invention in the presence of anions. In another embodiment, this invention is directed to positively charged polyselenophenes comprising monomers A of Formula 26-40. In another embodiment, the polyselenophene dispersion is in water. In another embodiment, the polyselenophene dispersion is in alcohol. In another embodiment, the polyselenophene is in water, alcohol or combination thereof.

In another embodiment, the anions are anions of polymeric carboxylic acids, for example, not limited to, polyacrylic acids, polymethacrylic acids or polymaleic acids. In another embodiment, the anions are anions of polymeric sulfonic acids, for example, not limited to, polystyrene sulfonic acid or polyvinyl sulfonic acid. In another embodiment the anions are anions of poly(toluenesulfonic acid), poly(4-ethylbenezene)sulfonic acid, poly(camphor-sulfonic acid), poly(tetradecyl-sulfonic acid), poly(dodecyl-sulfonic acid), poly(methane-sulfonic acid), poly(naphthalene-sulfonic acid), poly(trifluoromethanesulfonic acid), or combination thereof. These polycarboxylic and polysulfonic acids may also be copolymers of vinyl carboxylic and vinyl sulfonic acids with other polymerizable monomers, such as acrylates and styrene. In another embodiment, the anion is tosylate, acrylate, maleate, sulfonate, p-toluenesulfate, 4-ethlybenzenesulfonate, camphor-sulfonate, tetradecyl-sulfonate, dodecyl-sulfonate, methane-sulfonate, naphthalene sulfonate, triflate, or any combination thereof.

In one embodiment, this invention provides a process for the preparation of polyselenophene dispersion comprising reacting a selenophene compound of this invention in the presence of an oxidant, and an acid or polyacid for between 20-36 h at a temperature of between 20-30° C. In another embodiment, the process for the preparation of polyselenophene dispersion comprises reacting a selenophene compound of formula 1 (R1=H), 8-11, 12a-12e, 12f (X═H), 18-24, 42 (X═H), 43 (X═H), 44 (X═H) in aqueous medium in the presence of an oxidant, an acid or poly-acid, in a molar ratio of the selenophene compound and the acid/poly-acid in the range of about 1:2 to 1:5 for between 20-36 h at a temperature of between 20-30° C. In another embodiment, the molar ratio between the selenophene compound and the acid/poly-acid is about 1:2.5. In another embodiment, the molar ratio between the selenophene compound and the acid/poly-acid is about 1:3. In another embodiment, the molar ratio between the selenophene compound and the acis/poly-acid is about 1:4. In another embodiment, the molar ratio between the selenophene compound and the acid/poly-acid is about 1:5.

In one embodiment, this invention provides a process for the preparation of polyselenophene dispersion comprising reacting a selenophene compound of this invention in the presence of Fe(III) salt of an acid or Fe(III) salt of poly-acid for between 20-36 h at a temperature of between 20-30° C. In another embodiment the selenophene compound of this invention comprises compound 1 (R1=H), 8-11, 12a-12e, 12f (X═H), 18-24, 42 (X═H), 43 (X═H), 44 (X═H), or any combination thereof.

In another embodiment the process for the preparation of polyselenophene dispersion comprises the use of acid or poly-acid. In another embodiment the poly-acid is poly(styrene sulfonic acid) (PSSA). In another embodiment, the poly-acid is poly(toluenesulfonic acid). In another embodiment, the poly-acid is poly(4-ethylbenezene)sulfonic acid. In another embodiment, the poly-acid is poly(camphor-sulfonic acid). In another embodiment, the poly-acid is poly(tetradecyl-sulfonic acid). In another embodiment, the poly-acid is poly(dodecyl-sulfonic acid). In another embodiment, the poly-acid is poly(methane-sulfonic acid). In another embodiment, the poly-acid is poly(naphthalene-sulfonic acid). In another embodiment, the poly-acid is poly(trifluoromethanesulfonic acid). In another embodiment the poly-acid is polyacrylic acid, polymethacrylic acid, polymaleic acid, polystyrene sulfonic acid or polyvinyl sulfonic acid. In another embodiment, the poly-acid is a copolymer comprising poly-acids described herein above. In another embodiment the acid is tosylic acid, acrylic acid, maleic acid, sulfonic acid, p-toluenesulfonic acid, 4-ethlybenzenesulfonic acid, camphor-sulfonic acid, tetradecyl-sulfonic acid, dodecyl-sulfonic acid, methane-sulfonic acid, naphthalene sulfonic acid, trifluoromethanesulfonic acid, or any combination thereof.

In one embodiment, this invention provides a process for the preparation of polyselenophene dispersion comprising reacting a selenophene compound of this invention in the presence of an oxidant, and an acid/poly-acid. In another embodiment, the oxidant is Fe(III) salt, for example not limiting to FeCl₃, Fe(ClO₄)₃ and iron(III) salts of organic acids and inorganic salts. In another embodiment, the oxidant is alkali or ammonium persulfates, alkali perborates, and copper salts, such as copper tetrafluoroborate. In another embodiment, the oxidant is H₂O₂. In another embodiment, the oxidant is sodium persulfate. In another embodiment, the oxidant is iron(III) sulfate added. In another embodiment, the oxidant is a combination thereof. In another embodiment, the oxidant is a combination of iron(III) sulfate and sodium persulfate. In another embodiment, the oxidant is air or oxygen, optionally in the presence of catalytic quantities of metal ions, such as iron, cobalt, nickel, molybdenum and vanadium ions.

In another embodiment, the process for the preparation of polyselenophene dispersion comprises stirring for 12-24 h. In another embodiment, the reaction is stirred for 24-30 h. In another embodiment, the reaction is stirred for 30-36 h.

In another embodiment, the process for the preparation of the polyselenophene dispersion is conducted at a temperature range of between 20-25° C. In another embodiment, the reaction is conducted at a temperature range of between 20-30° C. In another embodiment, the reaction is conducted at a temperature range of between 25-30° C. In another embodiment, the reaction is conducted at a temperature range of between 30-35° C. In another embodiment, the reaction is conducted at a temperature range of between 20-40° C.

In one embodiment, the selenophene dispersion of this invention is in water. In another embodiment, the selenophene dispersion is in alcohol. In another embodiment, the selenophene dispersion is in water, alcohol or combination thereof.

In another embodiment, poly(3,4-ethylenedioxyselenophene) and poly(styrene sulfonate)—(PEDOT:PSS) is prepared according to Example 25.

In one embodiment, the oxidation of the selenophene compound 1 (R1=H), 8-11, 12a-12e, 12f (X═H), 18-24, 42 (X═H), 43 (X═H), 44 (X═H) using sodium persulfate (Na₂S₂O₈) and iron(III) sulfate (Fe₂(SO₄)₃) initiate the polymerization process, resulting in wet-processable aqueous dispersion (gel particles).

In one embodiment the polyselenophene dispersion is applied to different substrates (e.g. glass, plastics) by spin-coating. The polyselenophene dispersion is applied to different substrates by annealing at 80-100° C. or by drying at room temperature (20-30° C.), wherein a conductive film is obtained. In another embodiment, the substrate is any solid surface, for example, not limited to glass, plastics, metals etc. . . . .

In one embodiment, the polymers of this invention are conducting polymers and the polyselenophene form a conductive film. In another embodiment, the conductive polymers and film of this invention have a work function less than or equal to 5.5 eV. In another embodiment the conductive polymers and film of this invention have a work function in the range of 4.8-5.5 eV. This allows for good hole injection from the anode into an adjacent semi-conductive hole transporter and/or emitter.

In one embodiment, the polyselenophene dispersion upon application on a substrate forms a transparent conducting electrode. In another embodiment, the polyselenophene dispersion have a conductivity of 10 S·cm⁻¹. In another embodiment, the polyselenophene dispersion of this invention have a conductivity in the range of between about 1-5 S·cm⁻¹. In another embodiment, the polyselenophene dispersion of this invention have a conductivity in the range of between about 5-10 S·cm⁻¹. In another embodiment, the polyselenophene dispersion of this invention have a conductivity in the range of between about 8-10 S·cm⁻¹. In another embodiment, the polyselenophene dispersion of this invention have a conductivity in the range of between about 10-12 S·cm⁻¹. In another embodiment, the polyselenophene dispersion of this invention have a conductivity in the range of between about 10-100 S·cm⁻¹. In another embodiment, the polyselenophene dispersion of this invention have a conductivity in the range of between about 10⁻²-1 S·cm⁻¹.

In another embodiment the polymers, dispersions and compounds of this invention upon application on a substrate provide semi-conducting properties.

In another embodiment, this invention provides an electronic device incorporating the compounds/polymers, dispersion or dimers of this invention, wherein such compounds/polymers, dispersion or dimers are incorporated within a photovoltaic device, an electrochromic device, an electrophoretic device, an organic thin film transistor, or an organic memory device.

In another embodiment, said electronic device is an organic light-emitting device, wherein the polymer or dispersion of this invention is incorporated in the device. In another embodiment the polymer is at least one of a hole injecting layer or a hole transporting layer. In another embodiment, the selenophene dispersion of this invention may be used as an alternative for ITO (indium tin oxide).

In one embodiment, a transistor is comprised of the compounds/polymers/dispersions or dimers of this invention, making use of their conductivity. In another embodiment the compounds/polymers, dispersions or dimers of this invention are used as electron hole conducting layers. In another embodiment the compounds/polymers/dispersion or dimers of this invention are used as active semiconductor. In another embodiment the compounds/polymers/dispersion or dimers of this invention are used as light absorption and converting units.

In one embodiment, this invention provides an organic light-emitting device, comprising: a first electrode; a second electrode; an emitting layer interposed between the first electrode and the second electrode; and at least one of a hole transporting layer and a hole injecting layer interposed between the emitting layer and the first electrode, said at least one of the hole transporting layer and the hole injecting layer obtained from a said conducting polymer. In another embodiment the layers are comprised of the polymers of the invention.

In one embodiment, there is provided an electrical device, for example, an opto-electronic device, comprising a conductive polymer/dispersion of this invention. In another embodiment, the electrical device comprises an anode, a cathode, and an organic semi-conductive layer between the anode and cathode. The conductive polymer may be provided in a layer between the anode and cathode. When the conductive polymer is used as a hole injection material, the layer comprising the conductive polymer is preferably located between the anode and the organic semi-conductive layer. When the conductive polymer is used as an electron transport material, the layer comprising the conductive polymer is preferably located between the cathode and the organic semi-conductive layer or in the organic semi-conductive layer. The organic semi-conductive layer preferably is light-emissive. The anode preferably comprises indium-tin-oxide (ITO).

In one embodiment, there is provided an electrical device, for example, an opto-electronic device, comprising a conductive polymer/dispersion of this invention as a charge injecting layer in light emitting devices; as a component in electrochromic displays and as electrodes in field-effect transistors and as photovoltaic cells as the alternative for ITO.

In another embodiment, the devices of this invention comprising the compounds/polymers/dispersion or dimers of this invention can be used in, e.g. imaging and electronics applications. In another embodiment, the devices can be used as a field effect transistor, light emitting diode, photovoltaic cell, or as display backplanes.

The organic semi-conductive layer may comprise one or more of a hole transporter, an electron transporter and a light emissive material. One or more further organic semi-conductive layers may be provided between the anode and cathode. One or both of the anode and cathode independently may comprise the conductive polymer composition.

In one embodiment, the conductive polymer or dispersion composition is deposited on electrical device by spin coating or ink jet printing. The conductive polymer may be deposited in an aqueous solution. The composition may be heated after being deposited so as to cross-link the polyelectrolyte. This heating step may be performed prior to deposition of an overlying layer.

In one embodiment, the conductive polymer/dispersion is in a form suited for deposition by ink jet printing techniques.

Spin-coating is particularly suitable for devices wherein patterning of the electroluminescent material is unnecessary—for example for lighting applications or simple monochrome segmented displays.

Inkjet printing is particularly suitable for high information content displays, in particular full color displays. Inkjet printing of OLEDs is described in, for example, EP 0880303, which is incorporated herein by reference.

In one embodiment, the conductive polymer/dispersion of this invention can be tuned with regard to conductivity by changing the matrix and/or the salt so as to change the strength of interaction between the matrix and salt ions thus changing conductivity. However, it is desirable that the matrix be water soluble which can limit the types of material which may be used as a matrix. PEG is a classical solid electrolyte.

In one embodiment, if multiple layers of the device are formed by solution processing then the skilled person will be aware of techniques to prevent intermixing of adjacent layers, for example by crosslinking of one layer before deposition of a subsequent layer or selection of materials for adjacent layers such that the material from which the first of these layers is formed is not soluble in the solvent used to deposit the second layer.

Alternatively, one layer is preferably formed by deposition from solution followed by heat treatment in order to render it substantially insoluble in the solvent used for deposition for a subsequent layer. In this way, cross-linking may be avoided.

In one embodiment, the polymers/dimers/dispersion and radialene of this invention are used in electrochromic devices wherein said polymers have high coloration efficiencies. In another embodiment the coloration efficiency is between 500-800 cm²/C. In another embodiment, the coloration efficiency is between 700-850 cm²/C. In another embodiment, the coloration efficiency of polymer 53 wherein A is a monomer of formula 12 is 773 cm²/C.

The following examples are presented in order to more fully illustrate the preferred embodiments of the invention. They should in no way, however, be construed as limiting the broad scope of the invention.

### EXAMPLES

### Example 1

### Synthesis of 3,4-dimethoxyselenophene

SeCl₂ was prepared by adding SO₂Cl₂ (5.4 g, 40 mmol) to selenium powder (3.2 g, 40 mmol) at 10-20° C. After 20 min, 10 mL hexane was added to it and the resulting reaction mixture was stirred for 3 h at room temperature. A clear brown solution of SeCl₂ was formed. (a) Maaninen, A.; Chivers, T.; Parvez, M.; Pietikaeinen, J.; Laitinen, R. S. Inorg. Chem. 1999, 38, 4093. (b) Zade, S. S.; Panda, S.; Singh, H. B.; Wolmershäuser, G. Tetrahedron Lett. 2005, 46, 665)

To a well stirred mixture of 2,3-dimethoxy-1,3-butadiene (3) (Pozzo, J-L.; Clavier, G. M.; Colomes, M.; Bouas-Laurent, H. Tetrahedron, 1997, 53, 6377) (4.0 g, 35 mmol) and CH₃COONa (8.2 g, 100 mmol) in hexane (240 mL) at −78° C. (dry ice/acetone bath) under an inert atmosphere was added a solution of freshly prepared SeCl₂ in hexane. The resulting yellowish solution was further stirred for 1 h at −78° C. and then removed from the cooling bath and the reaction mixture was brought to room temperature over a period of 1 h and further stirred for 4 h. The reaction mixture was filtered through neutral silica gel and washed with hexane. The residue was concentrated to give a light yellow oil. The crude product was purified by recrystallization in hexane at low temperature to furnish 4 (2.80 g, 42%) as a white crystalline solid. mp. 43-45° C.; ¹H NMR (250 MHz, CDCl₃) δ 6.55 (s, 2H), 3.85 (s, 6H); ¹³C NMR (62.5 MHz, CDCl₃) δ 148.9, 96.0, 57.0; MS EI (70 eV), m/z: 192 (M⁺, 100%), 149, 134, 93; Anal calcd. for C₆H₈O₂Se: C, 37.71; H, 4.22. Found. C, 38.05; H, 4.23. ¹H NMR spectrum of 4 was in good agreement with the reported data. (Dian, G.; Merlet, N.; Barbey, G. J. Electroanal. Chem. 1987, 238, 225-237). ⁷⁷Se NMR (250 MHz, CDCl₃) δ=377.9 ppm.

### Example 2

### Synthesis of 3,4-ethylenedioxyselenophene (EDOS; 39)

A solution of 4 (500 mg, 2.62 mmol), with 6 equivalents of ethylene glycol and a catalytic amount of p-toluene sulfonic acid (150 mg) in dry toluene (100 mL) was stirred for 6 h at 50-55° C. The completion of the reaction was monitor by thin layer chromatography (TLC). Toluene was removed under reduced pressure and the residue was diluted with water (100 mL). The mixture was extracted with ether (3×50 mL). The combined organic layers were washed with dilute NaHCO₃ solution and brine and then concentrated. Purification of the crude residue by chromatography on silica gel (hexane) gave 5 (255 mg, 52%) as a colorless liquid. ¹H NMR (250 MHz, CDCl₃) δ 6.79 (s, 2H), 4.17 (s, 4H); ¹³C NMR (62.5 MHz, CDCl₃) δ 142.7, 101.7, 64.3. Spectral data of 5 were in agreement with the reported data for 3,4-ethylenedioxyselenophene (5) (Aqad, E.; Lakshmikantham, M. V.; Cava, M. P. Org. Lett. 2001, 3, 4283-4285). ⁷⁷Se NMR (250 MHz, CDCl₃) δ=374.9 ppm.

### Example 3

### Synthesis of 2,5-dibromo-3,4-ethylenedioxyselenophene

To a stirred solution of 5 (500 mg, 2.65 mmol) in CHCl₃ (25 mL) at 0° C. was added N-bromosuccinimide (NBS: 845 mg, 5.30 mmol). After stirring for 1 h at 0° C., the resulting reaction mixture was diluted with water (50 mL). The resulting aqueous layer was extracted with CHCl₃ (3×25 mL). The combined organic phase was washed with water followed by brine and then concentrated. Purification of the crude residue by chromatography on silica gel (hexane) afforded 6 (870 mg, 95%) as a white crystalline solid. ¹H NMR (250 MHz, CDCl₃) δ 4.24 (s); ¹³C NMR (62.5 MHz, CDCl₃) δ 140.6, 86.6, 64.6. MS EI (70 eV), m/z: 350.1, 348 (M⁺, 100%), 346.1, 187, 95. Single crystals of 2,5-dibromo-3,4-ethylenedioxyselenophene i.e DBEDOS (6) were grown from CHCl₃ solution at low temperature. The compound polymerized during melting point measurement or during elemental analysis.

### Example 4

### Synthesis of 2,5-diiodo-3,4-ethylenedioxyselenophene (DIEDOS)

To a stirred solution of 5 (420 mg, 2.20 mmol) in CHCl₃ (16 mL) and AcOH (8 mL) at 0° C. was added N-iodosuccinimide (NIS: 1.0 g, 4.40 mmol). The reaction mixture was brought to room temperature and further stirred for 4 h and then diluted with water (40 mL). The resulting aqueous layer was extracted with CHCl₃ (3×20 mL). The combined organic phase was washed with water followed by brine and then concentrated. Purification of the crude residue by chromatography on silica gel (hexanes: ethylacetate, 10:1) afforded 7 (810 mg, 83%) as a white crystalline solid. ¹H NMR (250 MHz, CDCl₃) δ 4.24 (s); ¹³C NMR (100 MHz, benzene) δ 144.9, 63.9, 51.8.

### Example 5

### Synthesis of hexyl substituted 3,4-ethylenedioxyselenophene (EDOS-C₆; 12)

A solution of 3,4-dimethoxyselenophene (250 mg, 1.31 mmol), 1,2-octandiol (400 mg, 2.74 mmol) and a catalytic amount of NaHSO₄ (50 mg) in dry toluene (35 mL) was stirred for 48 h at 70° C. The completion of the reaction was monitored by thin layer chromatography (TLC). Toluene was removed under reduced pressure and the residue was diluted with water (50 mL). The mixture was extracted with hexane (3×50 mL). The combined organic layers were washed with H₂O, brine, dried (MgSO₄), and then concentrated. Purification of the crude residue by chromatography on silica gel (60-230 mesh; hexane as eluents) gave EDOS-C₆ (255 mg, 72%) as a colorless liquid. NMR (250 MHz, CDCl₃) δ 6.76 (s, 2H), 4.14-4.04 (m, 2H), 3.84 (dd, J=11.7 Hz, 8.5 Hz, 1H), 1.71-1.48 (m, 2H); 1.42-1.24 (m, 8H), 0.89 (t, J=6.5 Hz, 3H); ¹³C NMR (62.5 MHz, CDCl₃) 143.1, 142.6, 101.2, 101.1, 73.4, 68.0, 31.6, 30.6, 29.1, 24.9, 22.5, 14.0; MS EI (70 eV), 274.3, 164.2.

### Example 6

### Synthesis of ethyl substituted 3,4-ethylenedioxyselenophene (EDOS-C₂; 12a)

EDOS-C₂ was prepared using 1,2-butanediol as described in Example 5. ¹H NMR (CDCl₃, 250 MHz), 1.03 (t, J=7.5 Hz, 3H), 1.60-1.70 (m, 2H), 3.83 (dd, J=11.25 Hz, J=8 Hz, 1H), 4.1 (dd, J=11 Hz, J=4 Hz, 1H), 3.95-4.06 (m, 1H), 6.73 (s, 2H). ¹³C NMR (C₆D₆, 400 MHz), 9.15, 23.60. 67.45, 74.30, 101.37, 101.41, 143.09, 143.53. MS (ESI). m/z 217.12.

### Example 7

### Synthesis of butyl substituted 3,4-ethylenedioxyselenophene (EDOS-C₄; 12b)

EDOS-C₄ was prepared using 1,2-hexanediol as described in Example 4. ¹H NMR (CDCl₃, 250 MHz). 0.91 (t, J=7 Hz, 3H), 1.34-1.361 (m, 6H), 3.83 (dd, J=8.5 Hz, J=3 Hz, 1H), 4.07 (dd, J=7 Hz, J=2 Hz, 1H), 4.01-4.10 (m, 1H), 6.73 (s, 2H). ¹³C NMR (C₆D₆, 400 MHz). 13.80, 22.33, 26.84, 30.19, 67.78, 73.16, 101.39, 101.41, 143.10, 143.56. MS (ESI). m/z 245.18.

### Example 8

### Synthesis of octyl substituted 3,4-ethylenedioxyselenophene (EDOS-C₈; 12c)

EDOS-C₈ was prepared using 1,2-decanediol as described in Example 4. ¹H NMR (CDCl₃, 250 MHz). 0.86 (t, J=6.25 Hz, 3H), 1.25-1.56 (m, 14H), 3.83 (dd, J=11.75 Hz, J=8.5 Hz, 1H), 4.09 (dd, J=11.75 Hz, J=2.25 Hz, 1H), 4.01-4.10 (m, 1H), 6.73 (s, 1H). ¹³C NMR (C₆D₆, 400 MHz). 14.10, 22.82, 24.96, 29.36, 29.50, 29.53, 31.98, 67.80, 73.19, 101.41, 101.44, 143.12, 143.57. MS (ESI). m/z 301.28.

### Example 9

### Synthesis of dodecyl substituted 3,4-ethylenedioxyselenophene (EDOS-C₁₂; 12d)

EDOS-C₁₂ was prepared using 1,2-tetradecanediol as described in Example 4. ¹H NMR (CDCl₃, 250 MHz). 0.86 (t, J=6.25 Hz, 3H), 1.24-1.56 (m, 21H), 3.83 (dd, J=11.5 Hz, J=8.5 Hz, 1H), 4.09 (dd, J=11.75 Hz, J=2.25 Hz, 1H), 4.01-4.09 (m, 1H), 6.73 (s, 2H). ¹³C NMR (C₆D₆, 400 MHz). 14.12, 22.87, 24.98, 29.54, 29.58, 29.62, 29.75, 29.86, 29.87, 29.89, 30.54, 32.09, 67.81, 73.19, 101.42, 101.44, 143.12. 143.57. MS (ESI). m/z 357.39.

### Example 10

### Synthesis of chiral hexyl substituted 3,4-ethylenedioxyselenophene (EDOS-C₆(c); 12e-chiral)

Chiral-EDOS-C₆ was prepared using chiral 1,2-octanediol as described in Example 4. ¹H NMR (250 MHz, CDCl₃) δ 6.75 (s, 2H), 4.02-4.14 (m, 2H), 3.83 (dd, J=11.7 Hz, 8.5 Hz, 1H), 1.70-1.50 (m, 2H); 1.40-1.20 (m, 8H), 9.50-8.50 (m, 3H).

### Example 11

### Chemical polymerization of 3,4-ethylenedioxyselenophene

Oxidative Polymerization of EDOS (5).

To a well stirred suspension of anhydrous FeCl₃ (650 mg, 4 mmol) in dry CHCl₃ (40 mL) at room temperature, a solution of 5 (150 mg, 0.8 mmol in 10 mL dry CHCl₃) was added dropwise. The resulting reaction mixture was warmed to 50° C. and further stirred for 18 h. at same temperature After cooling the reaction mixture at room temperature, MeOH (20 mL) was added The resulting dark black suspension was stirred for an additional 2 h at room temperature. The black solid formed was separated by filtration and then washed with MeOH. The black solid was further purified by repeated Soxhlet extraction with MeOH, acetone and hexane, respectively. The black solid was dried to furnish PEDOS (2) (120 mg) as a powder. Elemental analysis data C, 30.41; H, 2.05.

### Example 12

### Solid-state polymerization of 2,5-dibromo-3,4-ethylenedioxyselenophene

In a typical experimental procedure, crystalline DBEDOS (6) (300 mg) was placed in a 50 mL round bottle flask that was closed with stopper. The flask was heated at 50° C. for 24 h, during which period the original white color of the DBEDOS turned black. The sequence of color changes (from white→gray→dark black) of the material and the appearance of brown bromine vapor in the flask was indicative of the progress of the solid state polymerization. After completion of the reaction, the resulting black PEDOS was collected to afford the bromine-doped polymer in 250 mg (which results in doping level of 43% of Br₃⁻ per selenophene ring). Performing the reaction in an inert atmosphere (Ar or N₂) or in a vacuum-sealed vial did not have any apparent effect on the polymerization time or properties of the product. Elemental analysis data C, 28.53; H, 2.18.

### Example 13

### Solid-State Polymerization of DIEDOS

In a typical experimental procedure, crystalline DIEDOS (7) (200 mg) was placed in a 50 mL round bottle flask that was closed with stopper. The flask was heated at 80° C. for 3 days, during which period the original white color of the DIEDOS turned black. The appearance of iodine vapor in the flask was indicative of the progress of the solid state polymerization. After completion of the reaction, the resulting black PEDOS was collected to afford the iodine-doped polymer in 140 mg (which results in doping level of 32% of I₃⁻ per selenophene ring). Elemental analysis data C, 18.85; H, 1.44; I, 55.52.

### Example 14

### Transition metal catalyzed polymerization of 2,5-dibromo-3,4-ethylenedioxyselenophene

To a stirred solution of 2,2′-bipyridyl (100 mg, 64 mmol) in 10 ml DMF at room temperature were added 1,5-cyclooctadiene (66 mg, 0.08 mL, 60 mmol) and Ni(COD)₂ (180 mg, 65 mmol). A solution of DBEDOS (6) (200 mg, 57 mmol,) in 6 mL DMF was added dropwise to the resulting solution at room temperature and the reaction mixture was warmed to 60° C. The reaction mixture was stirred for 18 h at 60° C. to yield a black precipitate. The resulting mixture was then poured into 100 mL of MeOH and the black precipitate was separated by filtration followed by repeated washing with MeOH. The black solid was further purified by repeated Soxhlet extraction with MeOH, acetone and hexane respectively. The black solid was dried to furnish PEDOS (2) (100 mg) as a powder. Elemental analysis data C, 34.06; H, 2.45.

### Example 15

### De-doping of solid-state polymerized 2,5-dibromo-3,4-ethylenedioxyselenophene

To a suspension of well-ground powder of as-prepared solid-state synthesized PEDOS (250 mg) in CH₃CN (75 mL) at room temperature was added hydrazine hydrate (2.2 mL). The resulting mixture was stirred overnight at room temperature. The black solid was collected by filtration and purified by repeated Soxhlet extraction with acetone and hexane. Elemental analysis data C, 33.37; H, 2.15; N. 0.68.

### Example 16

### Electropolymerization and Electrochemical Measurements of Selenophenes and Polyselenophenes

Electropolymerization was carried out with a Princeton Applied Research VF263A potentiostat, employing a ITO-coated glass slides as the working electrode (7×50×0.6 mm, Rs=8˜12Ω/□, Delta Technologies Inc.), a gold flag as the counter electrode, and Ag/AgCl as the reference electrode. The electrolyte used was 0.1 M LiBF₄ in PC. The poly(alkyl-3,4-ethylnedioxyselenophenes) (PEDOS-Cₙ) films were prepared on ITO-coated glass with a size of 0.7 cm×3.2 cm in electrolyte at constant potentials of 0.93˜0.98 V from a 0.01 M monomer solution in 0.1 M LiBF₄/PC, which was bubbled with nitrogen to remove any incipient oxygen. Spectroelectrochemical data was recorded on a V-570 UV-vis-NIR spectrophotometer connected to a computer. A three-electrode cell assembly was used where the working electrode was ITO-coated glass slides, the counter electrode was a platinum wire, and Ag/AgCl was used as the pseudo-reference electrode. Thickness of films is measured by Dektak 6M Manual Veeco Instruments. Spectroelectrochemical and ED data for PEDOS-Cₙ films are shown in the following table.

| | | | | | CR | | Switching |
|---|---|---|---|---|---|---|---|
| | Eₒₙₛₑₜ^{a} | E_{1/2}^{b} | λₘₐₓ | E_{g} | ΔT | CE | time |
| Polymers | (V) | (V) | (nm) | (eV) | % | (cm²/C) | (s) |
| (A)n | −0.95 | −0.49 | 666 | 1.42 | 55 | 212 | 0.6 |
| A = monomer | | | | | | | |
| (38) | | | | | | | |
| PEDOS | | | | | | | |
| (A)n | −0.75 | −0.67 | 688 | 1.46 | 70 | 243 | 0.6 |
| A = monomer | | | | | | | |
| (40) Alk is ethyl | | | | | | | |
| PEDOS-C₂ | | | | | | | |
| (A)n | −0.52 | −0.48 | 761 | 1.54 | 77 | 369 | 0.5 |
| A = monomer | | | | | | | |
| (40) Alk is | | | | | | | |
| butyl | | | | | | | |
| PEDOS-C₄ | | | | | | | |
| (A)n | −0.55 | −0.46 | 763 | 1.54 | 88 | 773 | 0.6 |
| A = monomer | | | | | | | |
| (40) Alk is | | | | | | | |
| hexyl | | | | | | | |
| PEDOS-C₆ | | | | | | | |
| (A)n | −0.54 | −0.44 | 764 | 1.54 | 76 | 680 | 0.6 |
| (40) Alk is | | | | | | | |
| hexyl | | | | | | | |
| PEDOS-C_{6(chiral)} | | | | | | | |
| 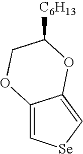 | | | | | | | |
| (A)n | −0.33 | −0.25 | 760 | 1.55 | 76 | 400 | 0.5 |
| A = monomer | | | | | | | |
| (40) Alk is octyl | | | | | | | |
| PEDOS-C₈ | | | | | | | |
| (A)n | −0.37 | −0.36 | 755 | 1.55 | 67 | 219 | 0.6 |
| A = monomer | | | | | | | |
| 40) Alk is | | | | | | | |
| dodecyl | | | | | | | |

^{a}the oxidation onset potentials of PEDOS-Cₙ films;
^{b}the redox potential E_{1/2} = (Eₚₐ + E_{pc})/2.

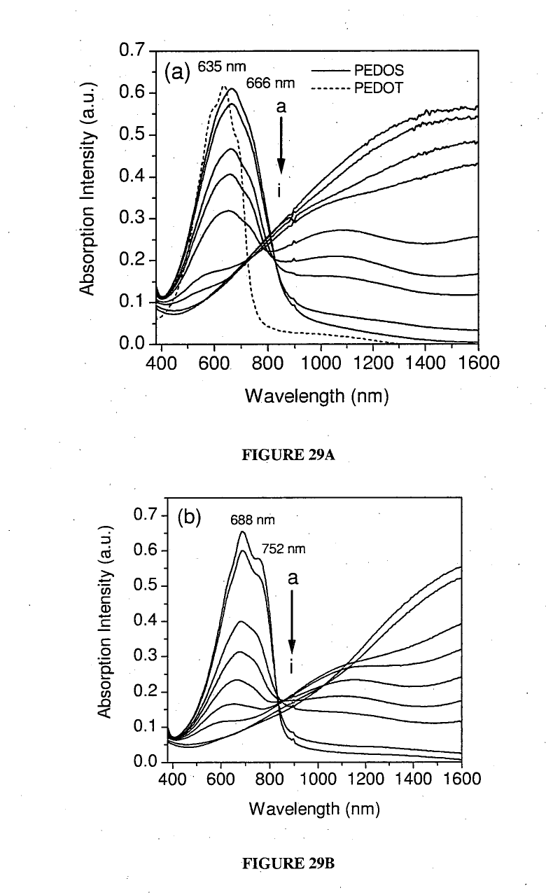

The alkyl-3,4-ethylnedioxyselenophenes (EDOS-Cₙ) (Compounds 12-12a-12e) can be electropolymerized quite rapidly and efficiently using constant potential of 0.93 V˜0.98 V vs. AgCl as shown in the above table to form the highly electroactive poly(alkyl-3,4-ethylnedioxyselenophenes) (PEDOS-Cₙ) films in 0.1 M LiBF₄/PC. FIG. 28 shows the CV date of PEDOS-Cₙ films in monomer-free electrolyte. The PEDOS film exhibits a broad redox peak extending from −0.84 V to −0.14 V and gives an oxidation onset potential of −0.95 V, which is lower than the oxidation onset potential at −0.55 V of PEDOT in 0.29 V. The increase of the length of alkyl chain in the side group PEDOS-Cₙ (n=2) gives rise to formation of narrow oxidation peak, preceded by a broad shoulder and followed by a capacitive current at more positive potential as shown in FIG. 28. At the same time, the PEDOS-C₂ film shows a redox peak

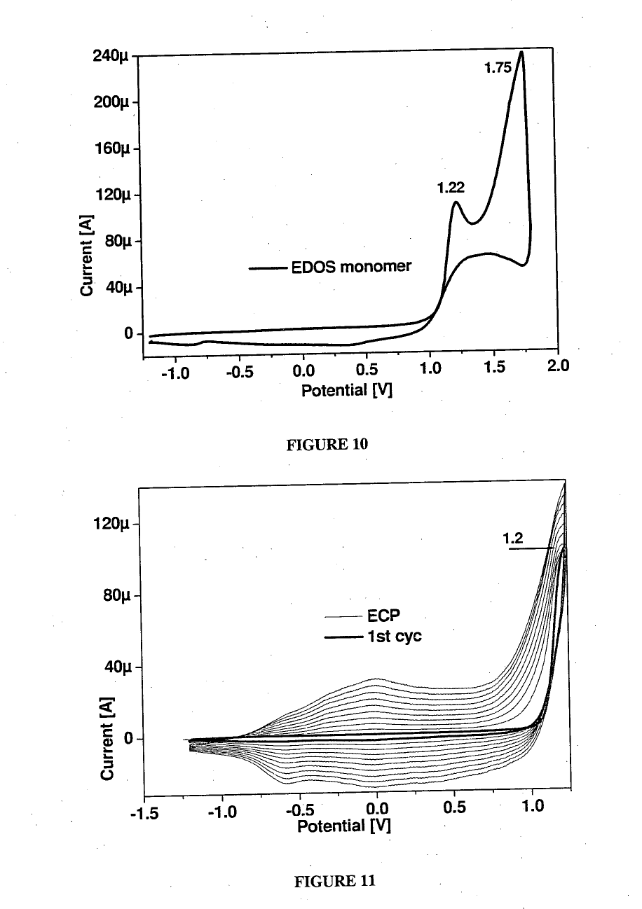

All electrochemical measurements were performed using a PAR Potentiostat model 263A in a standard three-electrode, one compartment configuration. The instrument was equipped with a Ag/AgCl wire, Pt wire and Pt disk electrode (dia. 1.6 mm BASi) serving as a pseudo reference, counter electrode and working electrode respectively. Pt disk electrodes were polished with alumina followed by sonication and further electropolished in 0.5M HClO₄ by cycling between from −0.23 to 1.25 V vs. Ag/AgCl saturated NaCl electrode (BASi). The electrolytic medium contained anhydrous acetonitrile and 0.1 M tetrabutylamonium perchlorate (TBAPC) as the electrolyte. All electrochemical solutions were purged with dry N₂ for a minimum of 15 minutes. Under these conditions Fc/Fc+ shows 0.34 V. Monomer concentration is about ˜20 μmol 3,4-ethylenedioxyselenophene (2 μl in 3 ml, d=1.802 gr/ml). After polymerization, the poly(3,4-ethylenedioxyselenophene) coated electrodes were washed with dichloromethane to remove soluble oligomers. The results of these studies are illustrated in FIGS. 10-13 and FIG. 29.

### Example 17

### Spectroelectrochemistry

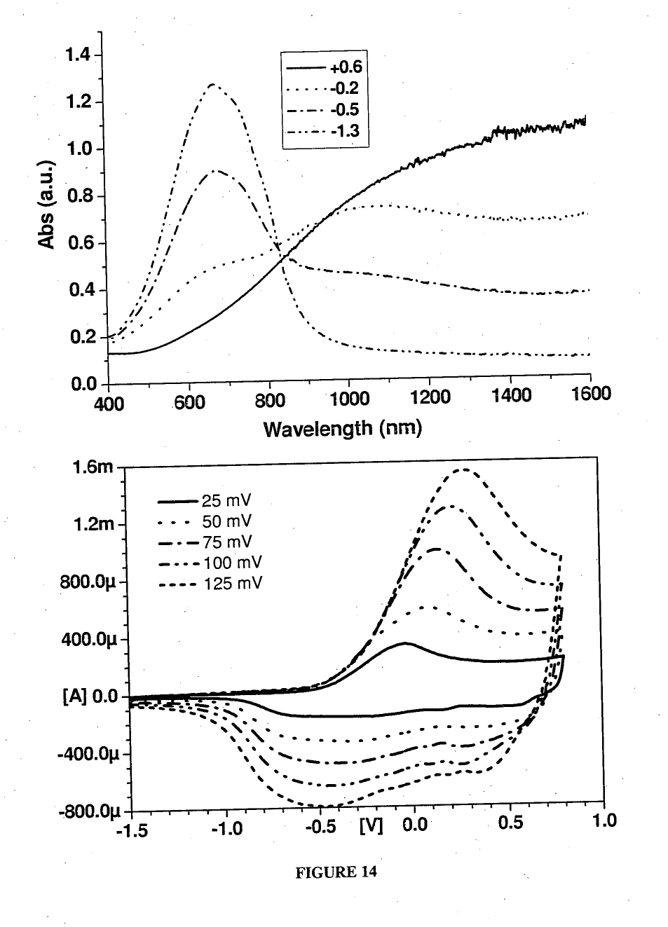

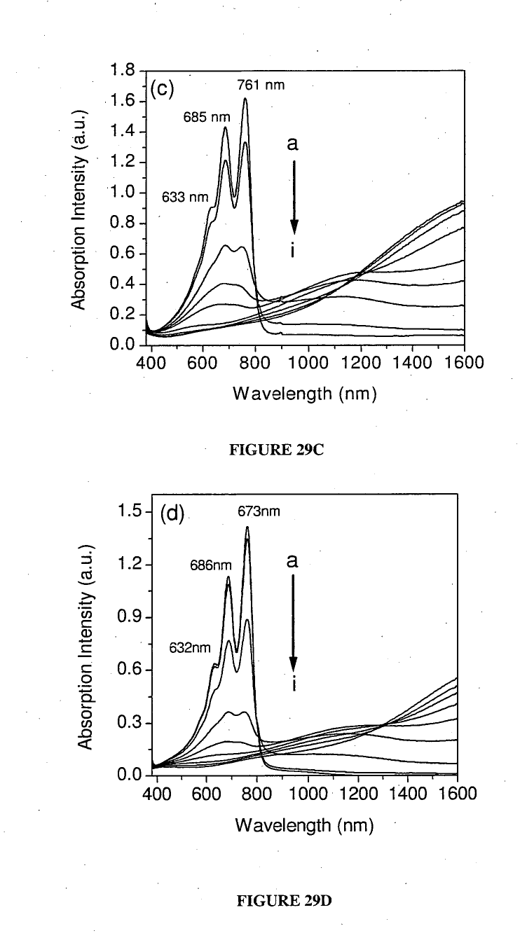

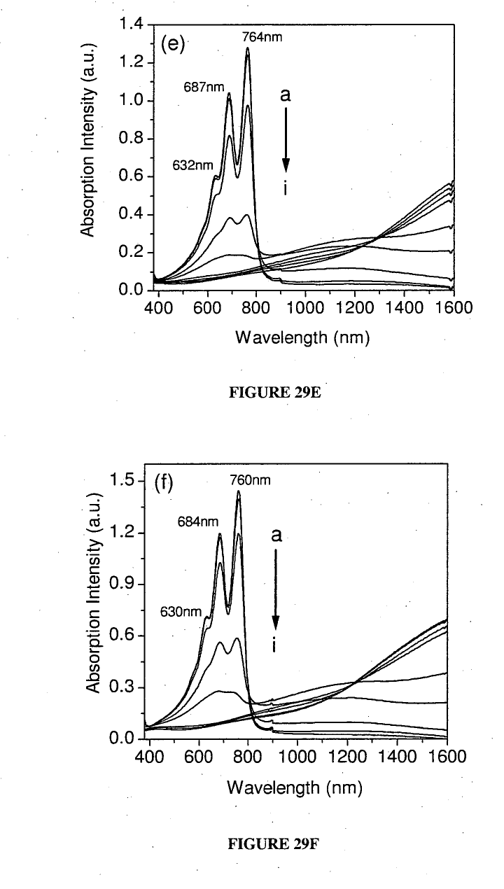

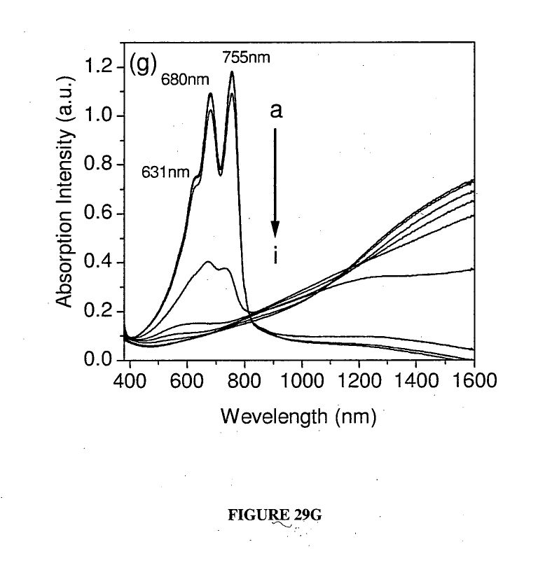

Spectra were measured using a UV-IR quartz optical cell (100-QX, Hellma) with a JASCO V-570 UV-VIS-NIR spectrophotometer. The optical spectrum of the polymer was obtained using indium tin oxide (5-15Ω, Delta Technologies, Stillwater) coated glass as a working electrode. The rest of the electrical set up was as specified in Example 14. Films were electrodeposited in 0.1M tetrabutylammonium perchlorate in propylene carbonate (PC) and ˜20 μmol by CV −0.5 to 1.2 V at 50 mV/s for 15cyc terminated at 0.8 V (stable oxidized state). Films were washed with dichloromethane and placed under vacuum until needed. Under these conditions Fc/Fc+ shows 0.34 V. The results of this study are illustrated in FIG. 14 and comparative data for PEDOS-Cₙ, are illustrated in FIG. 29.

### Example 18

### Conductivity Measurements

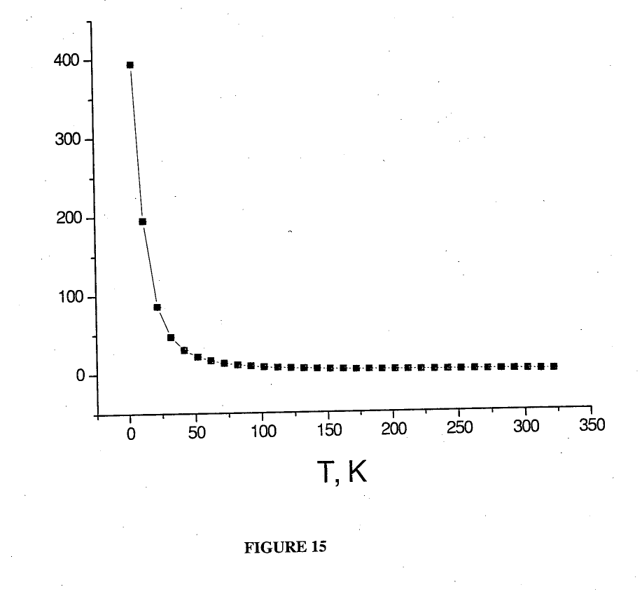

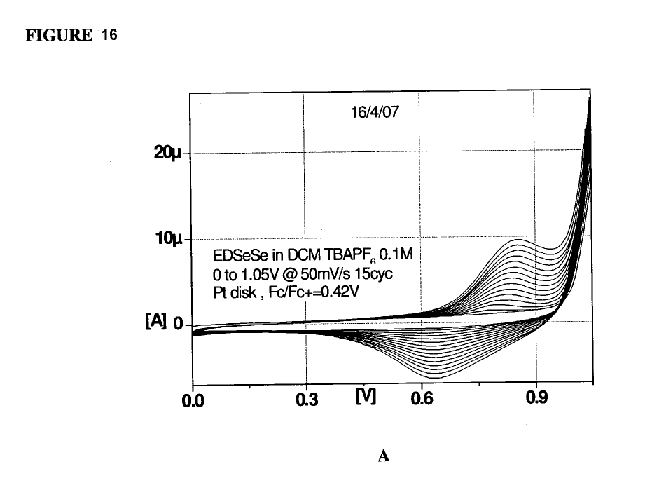

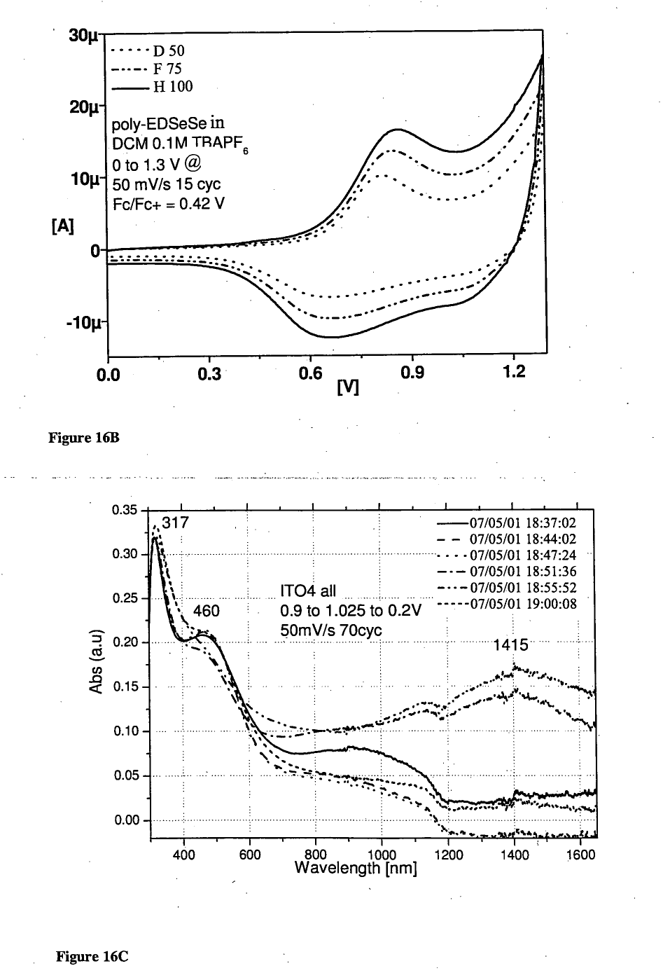

The conductivity was measured by two point probe method. Samples were pressed within glass tube of 2 mm and the samples lengths were 1 to 2 mm. Standard multimeter (F115, FLUKE company) was used. In some cases conductivity was measured by four point probe method. In these instances the samples were cooled at down about 10 Kelvin. Usual temperature dependence for semiconductor was observed. The results of such measurement are given in FIG. 15.

### Example 19

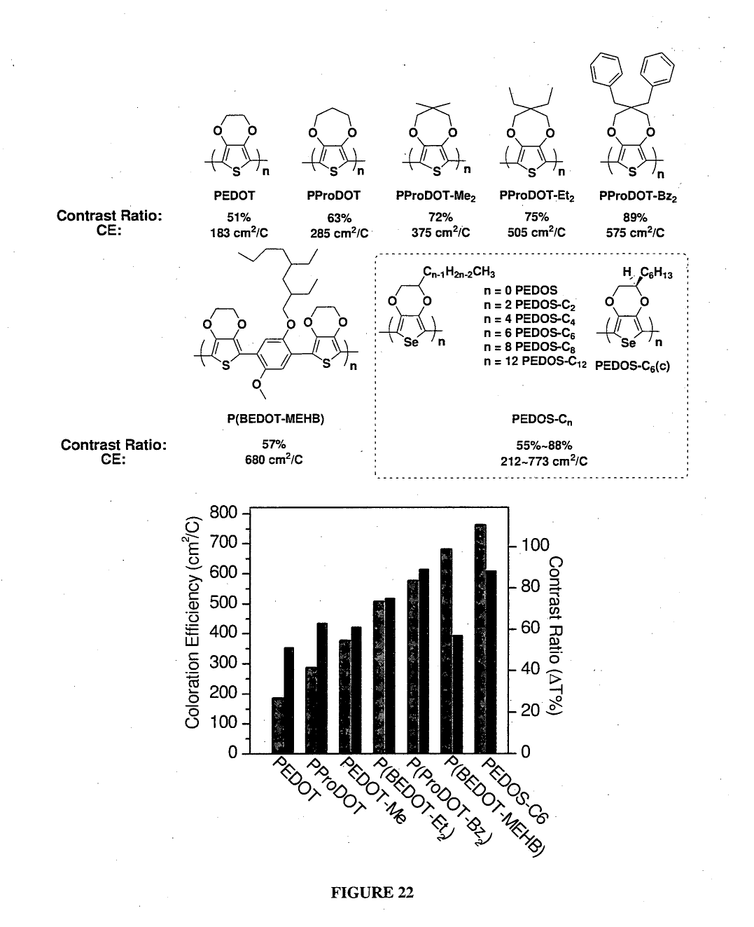

### Comparison of the electrochromic properties of poly(3,4-ethylnedioxyselenophenes) (PEDOS) and poly(3,4-ethylnedioxythiophene) PEDOT (FIG. 22)

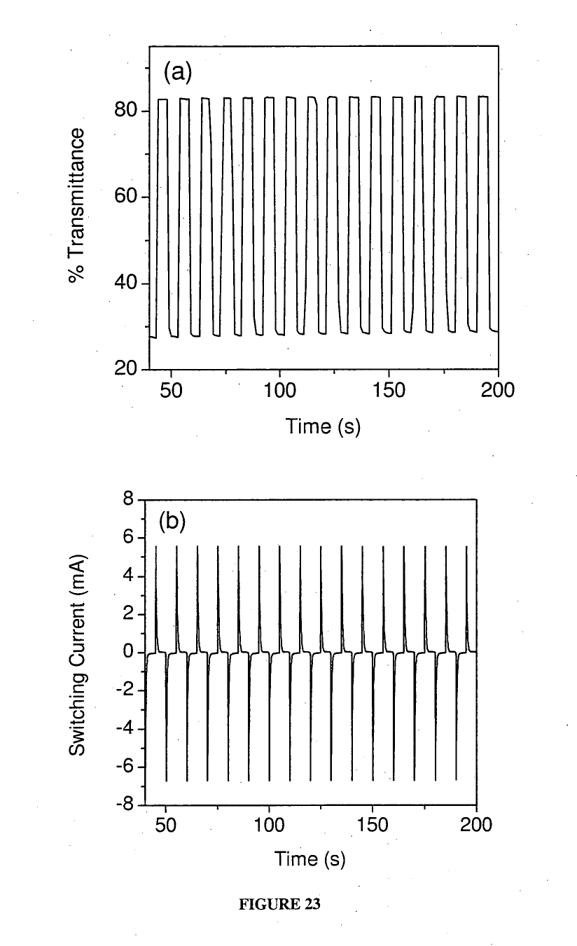

PEDOS films were electrodeposited onto indium-tin-oxide (ITO) coated glass having dimensions of 3.2 cm×0.7 cm at a constant potential of 0.93 V vs. Ag/AgCl and passing charge of 0.03 C to form a film with a thickness of ˜100 nm. PEDOS film obtained at those conditions has a band gap of ca. 1.42 eV (873 nm) as determined by the onset of the π-to-π* transition from the UV-vis-NIR spectrum. It also exhibits a high contrast ratio of 55% at 666 nm (λₘₐₓ) and a CE of 212 cm²/C (T_{b} %=82.51, T_{c} %=27.59 and Q_{d}=2.25 mC/cm² based on the data in FIG. 23). The measured bleached time and the colored time are 0.4 s and 0.6 s when the contrast ratio reaches 95% of its maximum value. Thus, both the contrast ratio and the coloration efficiency of PEDOS are better than those of PEDOT, which has a contrast ratio of 51% and a CE of 183 cm²/C at 95% of its maximum contrast ratio (FIG. 22).

### Example 20

### Spectroelectrochemical measurements for a poly(hexane-3,4-ethylnedioxyselenophenes) PEDOS-C₆ film

EDOS-C₆(compound 12) was electropolymerized onto ITO-coated glass using a constant potential of 0.98 V vs. Ag/AgCl in 0.1 M LiBF₄/propylene carbonate (PC). As shown in FIG. 29(d), a series of spectra were collected from the resultant PEDOS-C₆ film at various potentials ranging from −0.9 V to 0.5 V. At an applied potential of −0.9 V, the neutral form of the polymer shows a distinctive π to π* interband transition which is split into two sharp peaks at 686 nm and 763 nm (λₘₐₓ) and one shoulder peak at 632 nm. The splitting is attributed to vibronic coupling, which suggests a high degree of regularity along the polymer backbone. The band gap (E_{g}), defined as the onset of the π to π* transition of the neutral polymer, was calculated to be 1.54 eV (805 nm) for PEDOS-C₆, which is about 0.12 eV higher than for PEDOS. The calculated (PBC/B3LYP/6-31G(d)) band gap for PEDOS-C₆ is 1.64 eV, which is practically identical to the calculated band gap of PEDOS (1.66 eV). So, the observed experimental difference of 0.12 eV between the band gaps of PEDOS and PEDOS-C₆ that was obtained from the onset of the UV absorption is a result of the sharper UV spectrum of PEDOS-C₆ relative to PEDOS due to the significantly higher solid state order and/or rigidity in PEDOS-C₆. As the applied potential increases, the absorption peaks at 686 nm and 763 nm decrease while the polaron (˜1100 nm) and bipolaron peaks (which peak in the NIR beyond the limits of the spectrophotometer) increase. At a higher doping level, the polaron peak reaches a maximum intensity and then begins to decrease, while the bipolaron peak continues to increase. Importantly, PEDOS-C₆ film switches between an absorbing dark blue neutral state and a highly transimissive and nearly colorless oxidized state.

### Example 21

### Transmittance measurements for a poly(hexane-3,4-ethylnedioxyselenophenes) (PEDOS-C₆) film

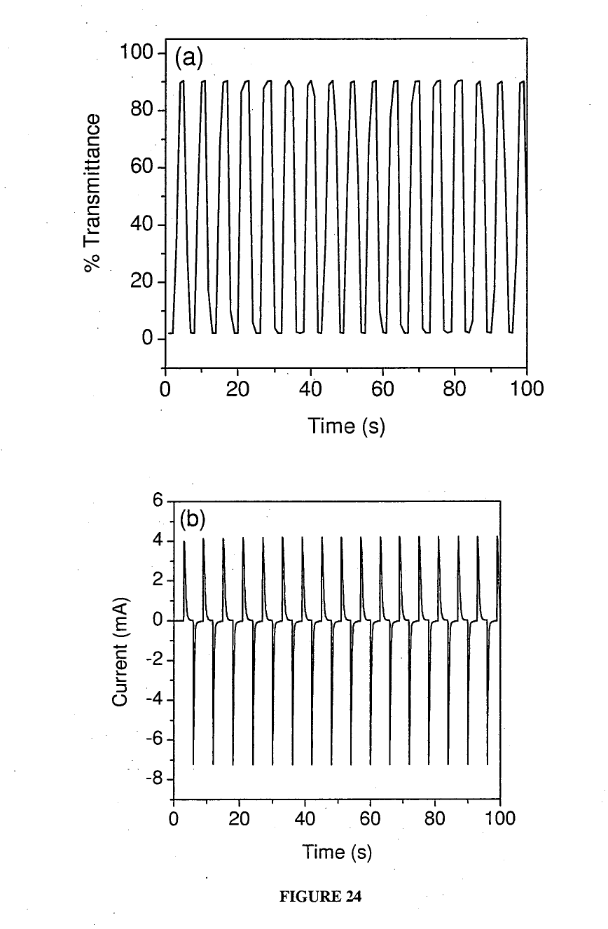

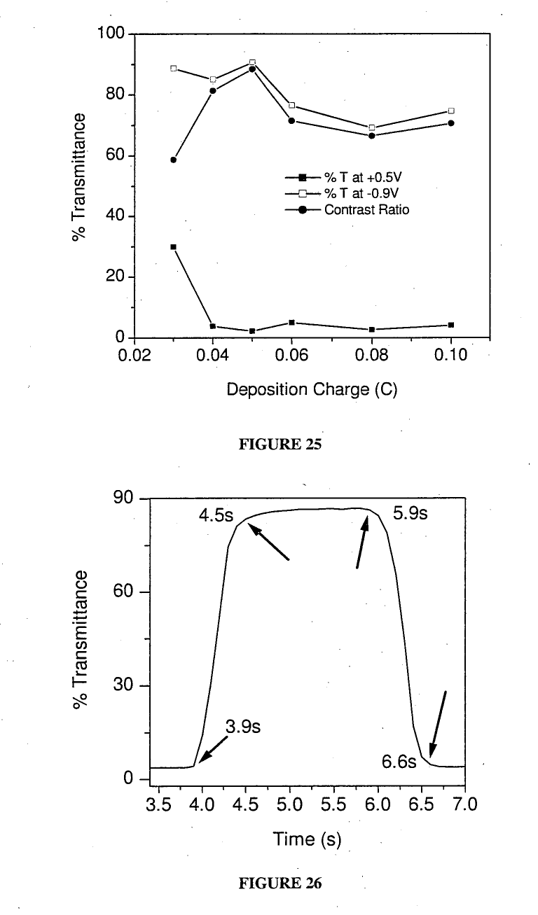

PEDOS-C₆ films obtained through electropolymerization at increasing deposition charges (of 0.03, 0.04, 0.05, 0.06, 0.08 and 0.1 C) at a constant potential of 0.98 V vs. Ag/AgCl, were switched between −0.9 and 0.5 V vs. Ag/AgCl with 3 s step intervals (in 0.1 M LiBF₄/PC on ITO-coated glass). Transmittance values at 763 nm as a function of each film's electropolymerization charge are given in FIG. 25. While the films were switched, the percentage transmittance (% T) at λₘₐₓ (763 nm) was simultaneously monitored as a function of time (FIG. 24). The highest contrast ratio (Δ % T) was achieved using a charge of 0.05 C. Significantly, PEDOS-C₆ films exhibit an unusual combination of a very high contrast ratio, a record high CE, a fast switching time and excellent long-term stability. The Δ % T of PEDOS-C₆ films achieves 88% (see FIGS. 25 and 24a), which is only 1% less than the highest reported Δ % T for any electropolymerized material, i.e. that of PPropOT-Bz₂ (see FIG. 22). The measured bleached time and the colored time are 0.6 s when the contrast ratio reaches 95% of its maximum (FIG. 26). So, PEDOS-C₆ film has a switching time comparable with the alkyl PEDOT derivatives. Coloration efficiency is one of most important characteristics of EC materials. The CE value of PEDOS-C₆ on ITO-coated glass in solution achieves 773 cm²/C (T_{b} %=90.43, T_{c} %=2.23 and Q_{d}=2.08 mC/cm²), which is higher than the next highest known CE (that of PEDOT-MEHB) which has a considerably inferior contrast ratio (see FIG. 22). PEDOS-C₆ film is superior to PEDOS, PEDOT and PEDOT derivative films as an electrochromic material.

### Example 22

### Stability measurements for poly(hexane-3,4-ethylnedioxyselenophenes) (PEDOS-C₆) films

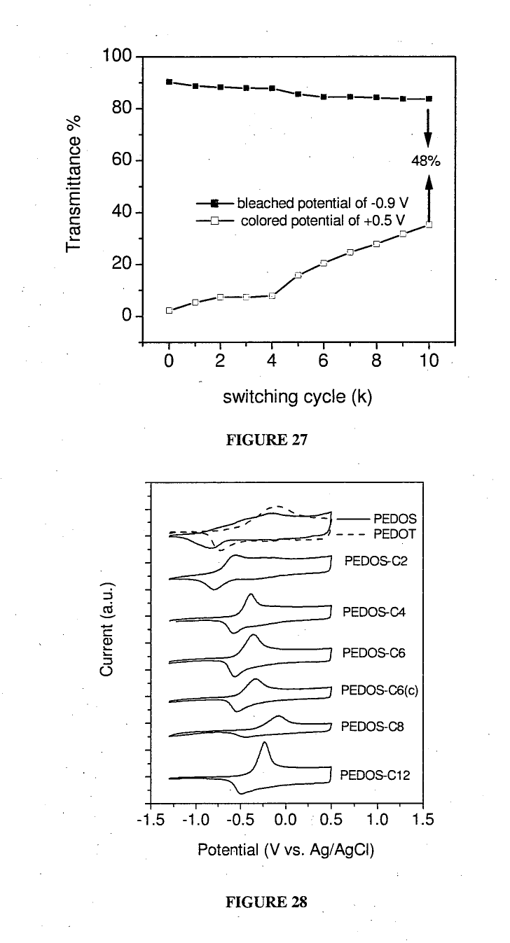

Stability measurements for PEDOS-C₆ films were carried out by measuring the contrast ratio as a function of number of switching cycles for the films on ITO-coated glass in an electrochemical cell opened to the air without purging using inert gas. The contrast ratio remains 48% after 10000 cycles at switching potentials between −0.9 V and 0.5 V with a 3 s interval (FIG. 27), indicating that PEDOS-C₆ films are highly stable.

### Example 23

### Preparation and Characterization of Radialene

Procedure for coupling reaction. To a stirred solution of 1,5-cyclooctadiene (0.6 mL) and Ph₃P (1.20 g, 4.58 mmol) in dry DMF (10 mL) was added Ni(COD)₂ (1.26 g, 4.58 mmol) at room temperature. A solution of 3,4-dibromothiophene (1.00 g, 4.13 mmol) in DMF (8 mL) was added dropwise to this mixture and stirred for 30 minute at room temperature. The resulting mixture was heated for 24 h at 70° C. After cooling, the reaction mixture was poured into ice-cold water (100 mL) and the products were extracted with CHCl₃ (3×60 mL). The combined organic layers were washed with water (50 mL), brine and then concentrated. Chromatographic purification on silica gel (hexane) yielded two compounds. The faster moving component is benzo[1,2-c:3,4-c′:5,6-c″]trithiophene (yield: 40%) and the slower-moving component is Cycloocta[1,2-c:3,4-c′:5,6-c″:7,8-c′″]tetrathiophene (yield: 40%).

Benzene[1,2-c:3,4-c′:5,6-c′]trithiophene (1a): white solid; mp. 237-238° C. (240° C. onset, according to DSC measurement, mp. 236-238° C.); ¹H NMR (250 MHz, CDCl₃) δ 7.67 (s, 6H); ¹³C NMR (62.5 MHz, CDCl₃) δ 132.3, 117.4; UV-vis (in CH₃CN): λₘₐₓ=317, 304, 254 and 247.

Benzene[1,2-c:3,4-c′:5,6-c′]triselenophene (1b):

This compound was prepared as a white solid from 3,4-dibromoselenophene, following the cyclooligomerization procedure adopted for the preparation of Benzene[1,2-c:3,4-c′:5,6-c′]trithiophene mp. 224° C. (decomposition); ¹H NMR (250 MHz, CDCl₃) δ 8.33 (s, 6H); ¹³C NMR (62.5 MHz, CDCl₃) δ 135.9, 122.8; UV-vis (in CH₃CN): λₘₐₓ=337, 322, 266 and 258.

Cycloocta[1,2-c:3,4-c′:5,6-c″:7,8-c′″]tetrathiophene (2a): white solid; mp. 298-299° C.; (300° C. onset, according to DSC measurement, mp. 300-301° C.); ¹H NMR (250 MHz, CDCl₃)δ 7.21 (s, 8H); ¹³C NMR (62.5 MHz, CDCl₃) δ 137.0, 124.4; UV-vis (in CH₃CN): λₘₐₓ=255.

Cycloocta[1,2-c:3,4-c′:5,6-c″:7,8-c′″]tetraselenophene (2b): This compound was prepared as a white solid from 3,4-dibromoselenophene following the cyclooligomerization procedure adopted for the preparation of compounds 4 and 6. mp. ˜230° C. (decomposition); ¹H NMR (250 MHz, CDCl₃) δ 7.82 (s, 8H); ¹³C NMR (62.5 MHz, CDCl₃) δ 140.9, 128.9; UV-vis (in CH₃CN): λₘₐₓ=271.

The following table provides yields of selected radialene compounds, prepared according to FIG. 7:

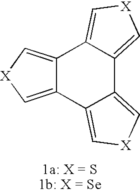

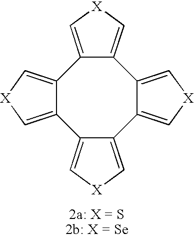

| X | Ligand | Yield (%) | Yield (%) |
|---|---|---|---|
| S | Ph₃P | 1a:40 | 2a:40 |
| S | 2,2′- | 1a:14 | 2a:40 |
| | bipyridyl | | |
| Se | Ph₃P | 1b:50 | 2b:10 |
| Se | 2,2′- | 1b:30 | 2b:5 |
| | bipyridyl | | |

Absorption Characteristics:

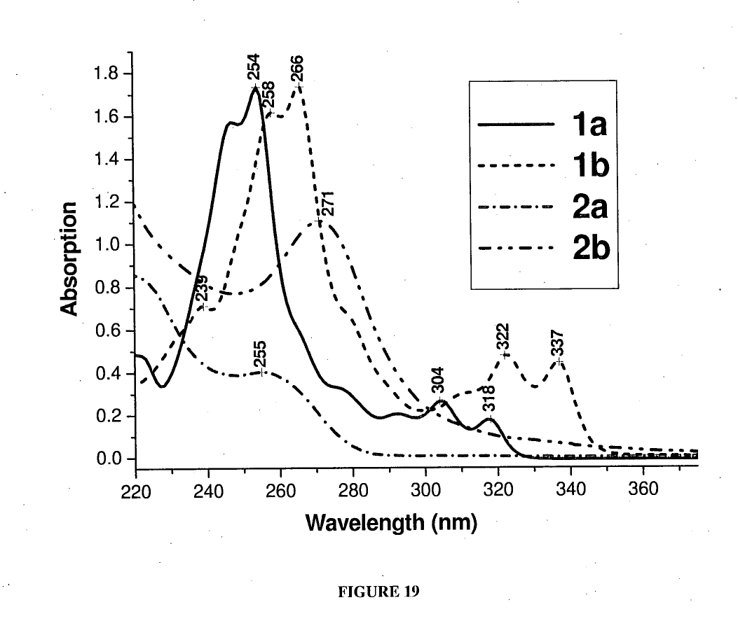

The UV spectra (FIG. 19) of compounds 1a, 1b, 2a and 2b show strong absorption peaks around 250-270 nm, which correspond to absorption by thiophene and selenophene units. The strongest absorption peak of compound 1a (254 nm) is blue shifted by 23 nm (0.49 eV) compared to the absorption of the parent thiophene (231 nm). A similar blue shift of 17 nm (0.31 eV) is observed for compound 1b (266 nm) relative to the parent selenophene (249 nm). These blue shifts result mostly from conjugation between heterocyclic rings in compounds 1a and 1b.

Cyclic Voltametry (CV) Results

All electrochemical measurements were performed using PAR Potentiostat model 263A in a standard three-electrode, one compartment configuration equipped with Ag/AgCl wire, Pt wire and a Pt disk electrode (dia. 1.6 mm from BASi) as the pseudo reference, counter electrode and working electrode, respectively. The cyclic voltametry experiments were performed in anhydrous acetonitrile solution with 0.1 M tetrabutylamonium perchlorate (TBAPC) as the supporting electrolyte with a scan rate of 10 mV/s. Under these conditions, the Fc/Fc⁺ couple was measured at the end of each experiment and all results were referenced to Fc/Fc⁺=0.37 V. All electrochemical solutions were purged with dry N₂ for at least 15 minutes.

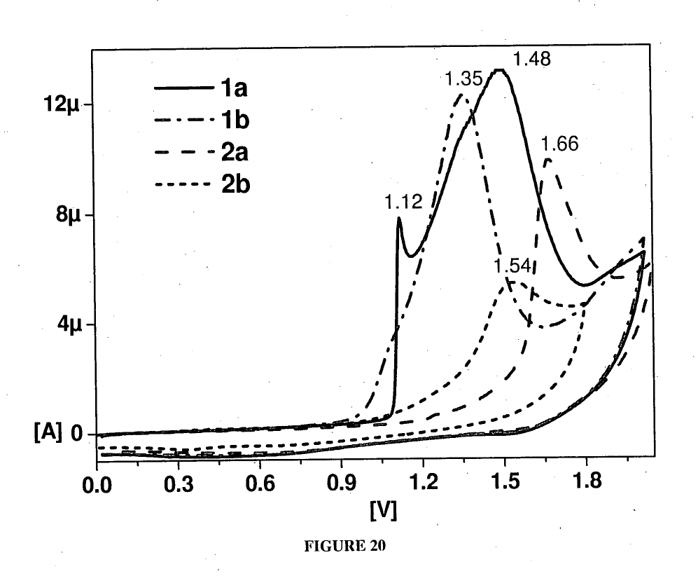

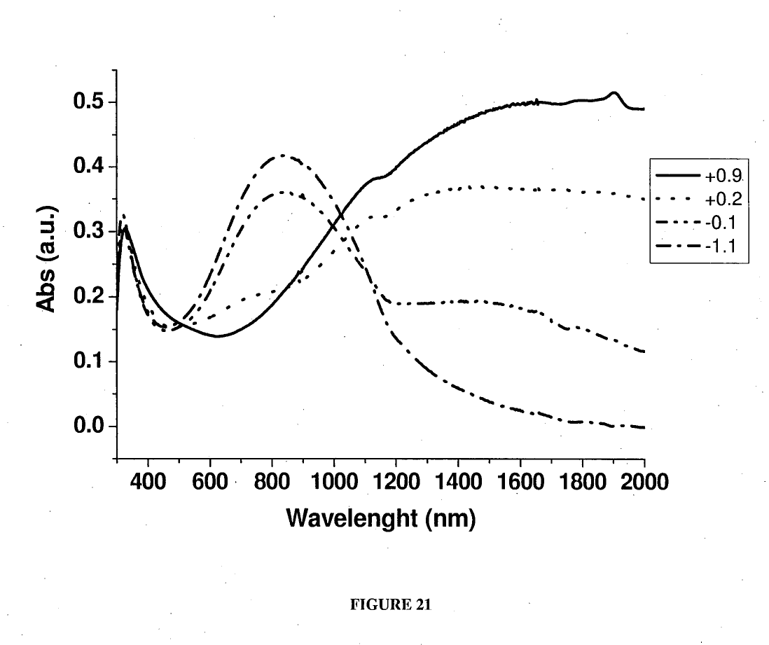

The redox behavior of compounds 1a, 1b, 2a and 2b has been studied by cyclic voltametry (CV) (FIG. 20). A reduction peak was not observed at potentials of up to −2V. All oxidation peaks are irreversible, which might indicate the oligomerization of these molecules under CV conditions. Radialenes 1a and 1b oxidized at potentials by about 0.2 V lower than their corresponding higher cyclooligomers 2a and 2b.

### Example 24

### Crystallization of Radialene

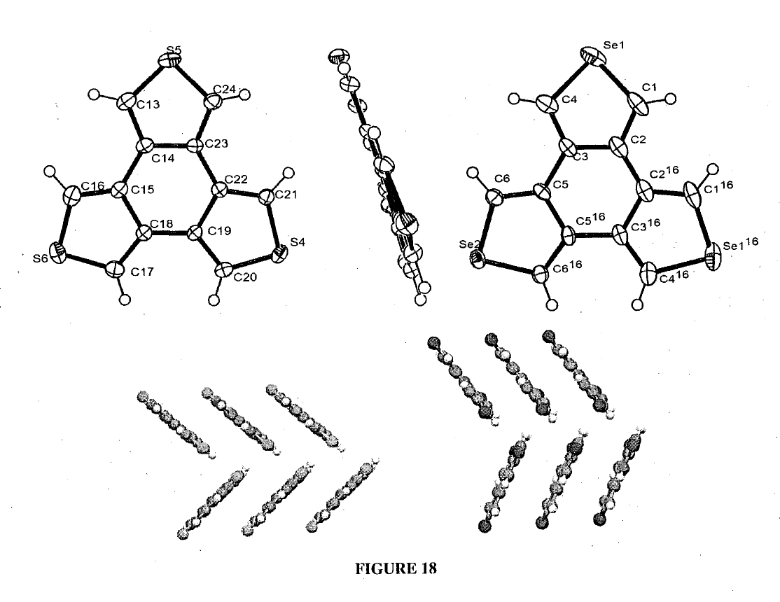

Single crystals of compounds 1a and 1b (as presented in Example 21) were grown by slow evaporation of chloroform solution at room temperature. X-ray structures of 1a and 1b are shown in FIG. 18 and experimental and calculated (B3LYP/6-31G(d)) structural data are given in Table 2. The calculated bond lengths are very close (within 0.01 Å, Table 2) to the experimental values Both 1a and 1b are practically planar (the average dihedral angle in the central six member ring is only 2.2° for compound 1a and 3.0° for compound 1b. The calculated (B3LYP/6-31G(d) optimized structures of 1a and 1b are planar, having D₃ₕ symmetry (confirmed by frequency analysis). Surprisingly, bond lengths in the central six member ring in both 1a and 1b are unusually long for aromatic C—C bonds, averaging 1.451 Å in the central ring of 1a, and 1.457 Å in the central ring of 1b. By comparison, the C—C bond length in benzene is significantly shorter, at 1.40 Å. Exocyclic double bonds are relatively short, at 1.371 Å and 1.363 Å in 1a and 1b respectively. All exocyclic bond lengths adjacent to the six member ring are within the C—C double bond range. Thus, it is clear that 1a and 1b correspond to the radialene family. Interestingly, in contrast to their parent analogs, [6]radialenes 1a and 1b have planar backbones.

Compounds 1a and 1b are well organized for good electron transport.

TABLE 2
Measured and calculated (at B3LYP/6-31G(d) and
are given in parenthesis)
bond lengths (Å) and bond angles (deg.) for 1a and 1b (the
values for compound 2 are given for comparison).

| | Bond lengths | | | | |
|---|---|---|---|---|---|
| | | | Exo- | | |
| | | | cyclic | Bond angle | |
| | Endo^{[a]} | Exo^{[a]} | C═C | Endo^{[b]} | Exo^{[b]} |
| 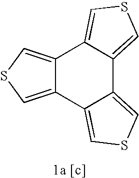 | 1.439 (1.449) | 1.463 (1.462) | 1.371 (1.373) | 112.0 (112.1) | 128.0 (127.9) |
| 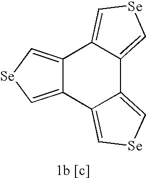 | 1.451 (1.453) | 1.463 (1.466) | 1.363 (1.370) | 114.3 (114.0) | 125.7 (126.0) |

^{[a]}An endo bond is one fused to the 5 member ring, while an exo bond is one connecting the two heterocyclic rings and exocyclic C═C bond is a bond exocyclic to the six member ring.
^{[b]}An endo bond angle is the C—C—C angle inside the heterocyclic ring, while the exo bond angle is outside the heterocyclic rings.
[c] Average measured values for three bonds or three bond angles are used. Measured values are within 0.01 Å or 1 deg. of the average value.
[[e] Average values from two different endo bonds or bond angles.

### Example 25

### Process for the preparation of poly(3,4-ethylenedioxyselenophene) and poly(styrene sulfonate)—PEDOS:PSS

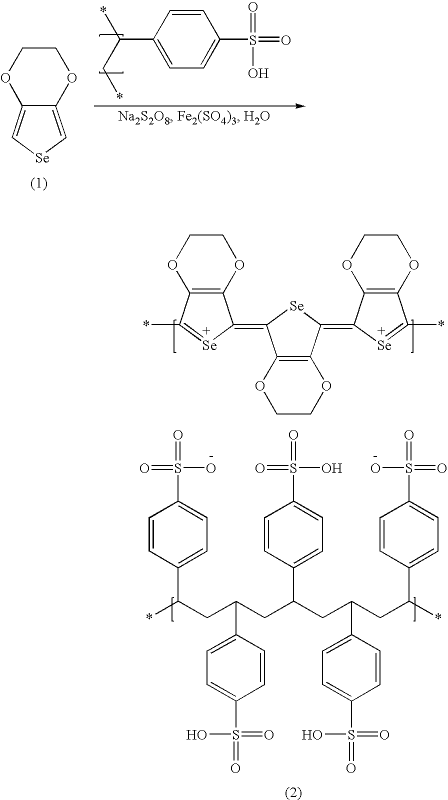

Sodium persulfate (0.21 g) was added with stirring to a solution of (0.12 g, 0.06 mmol) 3,4-ethylenedioxyselenophene (1) and 1.67 g poly(styrene sulfonic acid) (18% wt, Mw=4000) in 20 ml water, after 15 min 0.002 g iron(III) sulfate was added, followed by stirring for 24 hours at room temperature. Over this period, the composition became dark blue in color to obtain (2). The composition was coated onto a glass film substrate. The coated film was dried at 95° C.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A compound represented by the structure of formula (1): 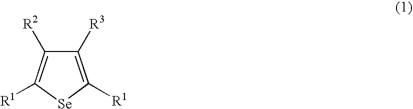
wherein:
R¹ is H, F, Cl, Br, I, SH, OSO₂CH₃ or OSO₂CF₃;
R² is H, F, Cl, Br, I, C₁-C₆alkyl, Y—H or Y—(C₁-C₆ alkyl) wherein if R³ is H then R² is not C₁-C₆ alkyl;
R³ is H, F, Cl, Br, I, C₁-C₆ alkyl, Z—H or Z—(C₁-C₆ alkyl) wherein if R² is H then R³ is not C₁-C₆ alkyl;
or
R² and R³ combine to form a 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];
or
R² and R³ combine to form Y-ring-Z having the following structure; 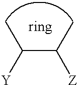
wherein said ring of Y-ring-Z is a cycloalkyl, heterocycloalkyl or aryl ring optionally substituted by 1-3 groups comprising halide, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];
R⁴ is C₁-C₆ alkyl;
R⁵ is C₁-C₆ alkyl;
Y is O, S, Se, PH, NR⁶ or C(R⁷)(R⁸);
Z is O, S, Se, PH, NR⁹ or C(R¹⁰)(R¹¹);
R⁶ is H, C₁-C₆ alkyl or C(O)(C₁-C₆ alkyl);
R⁷ is H, CN, C₁-C₆ alkyl, OH, SH, NH₂ or aryl;
R⁸ is H, CN, C₁-C₆ alkyl, OH, SH, NH₂ or aryl;
R⁹ is H, C₁-C₆ alkyl or C(O)(C₁-C₆ alkyl);
R¹⁰ is H, CN, C₁-C₆ alkyl, OH, SH, NH₂ or aryl;
R¹¹ is H, CN, C₁-C₆ alkyl, OH, SH, NH₂ or aryl;
wherein if R¹ is H, then R² and R³ are not OMe, do not form an unsubstituted [1,4] dioxane ring; and do not form a 5 membered unsaturated heterocyclic ring comprising Se or S.

2. The compound of claim 1 wherein R¹ is H, Cl, Br, I or SH.

3. The compound of claim 1 wherein R¹ is H or Br.

4. The compound of claim 1, wherein R¹ is Br, R² is Y—(C₁-C₆ alkyl), R³ is Z—(C₁-C₆ alkyl), Y is O and Z is O.

5. The compound of claim 4, wherein R² is Y—CH₃ and R³ is Z—CH₃.

6. The compound of claim 5, wherein said compound is represented by the structure of formula (2):

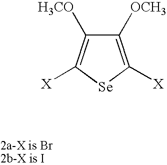

7. The compound of claim 1, wherein said 4-8 membered ring is optionally substituted with 1-3 groups comprising C₁-C₆ alkyl or (C₀-C₆ alkyl)-aryl;

8. The compound of claim 1 wherein R¹ is Br or I and R² and R³ combine to form a dioxy 4-8 membered ring containing 0-3 double bonds wherein said 4-8 membered ring is optionally substituted with 1-3 groups comprising C₁-C₁₂ alkyl or (C₀-C₆ alkyl)-aryl.

9. The compound of claim 8, wherein said compound is represented by the structure of formula (3)

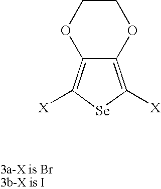

10. The compound of claim 8, wherein said compound is represented by the structure of formula (4):

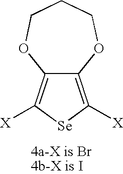

11. The compound of claim 8, wherein said compound is represented by the structure of formula (5):

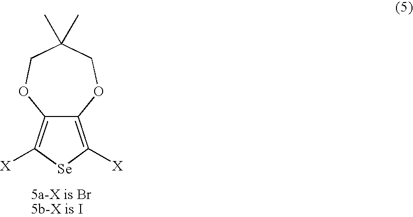

12. The compound of claim 1 wherein R¹ is H and R² and R³ are combined to form a dioxy 4-8 membered ring containing 0-3 double bonds wherein said 4-8 membered ring is optionally substituted with 1-3 groups comprising C₁-C₁₂ alkyl or (C₀-C₆ alkyl)-aryl and wherein, if R¹ is H, then R² and R³ do not form a [1,4] dioxane ring.

13. The compound of claim 12, wherein said compound is represented by the structure of formula (8):

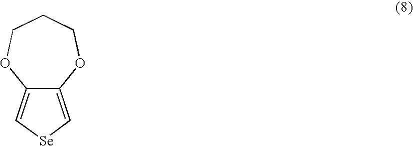

14. The compound of claim 12, wherein said compound is represented by the structure of formula (9):

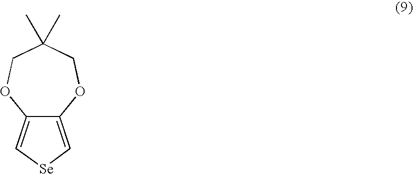

15. The compound of claim 12, wherein said compound is represented by the structure of formula (12e):

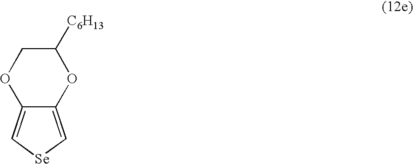

16. The compound of claim 12, wherein said compound is represented by the structure of 12(e) chiral an:

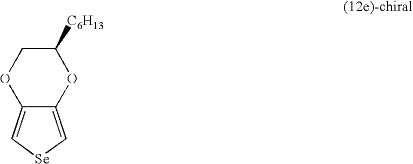

17. The compound of claim 12, wherein said compound is represented by the structure of formula (12a):

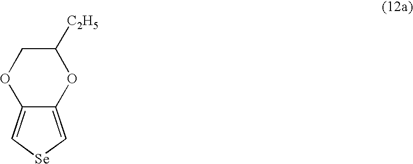

18. The compound of claim 12, wherein said compound is represented by the structure of formula (12b):

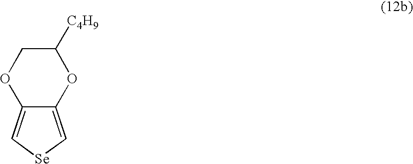

19. The compound of claim 12, wherein said compound is represented by the structure of formula (12c):

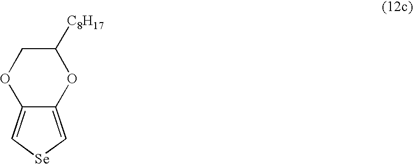

20. The compound of claim 12, wherein said compound is represented by the structure of formula (12d):

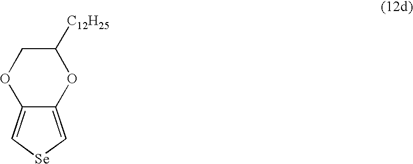

21. The compound of claim 1 wherein R¹ is Br or I and R² and R³ are combine to form a dithio 4-8 membered ring containing 0-3 double bonds wherein said 4-8 membered ring is optionally substituted with 1-3 groups comprising C₁-C₁₂ alkyl or (C₀-C₆ alkyl)-aryl.

22. The compound of claim 21, wherein said compound is represented by the structure of formula (13):

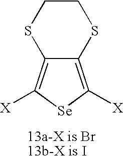

23. The compound of claim 21, wherein said compound is represented by the structure of formula (14):

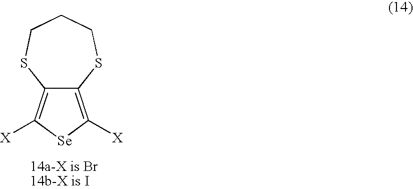

24. The compound of claim 21, wherein said compound is represented by the structure of formula (15):

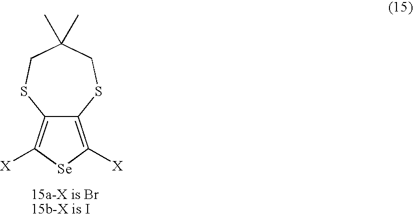

25. The compound of claim 1 wherein R¹ is H and R² and R³ are combine to form a dithio-4-8 membered ring containing 0-3 double bonds wherein said 4-8 membered ring is optionally substituted with 1-3 groups comprising C₁-C₁₂ alkyl or (C₀-C₆ alkyl)-aryl.

26. The compound of claim 25, wherein said compound is represented by the structure of formula (18):

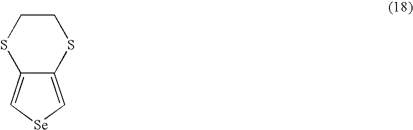

27. The compound of claim 25, wherein said compound is represented by the structure of formula (19):

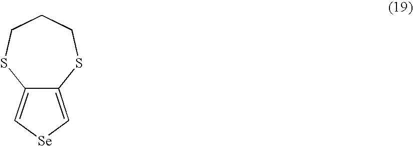

28. The compound of claim 25, wherein said compound is represented by the structure of formula (20):

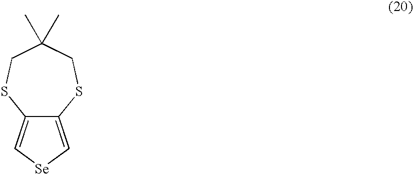

29. The compound of claim 25, wherein said compound is represented by the structure of formula (21):

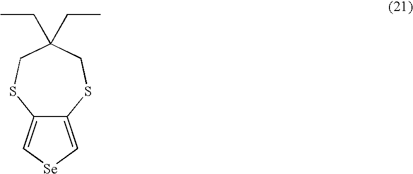

30. The compound of claim 1 wherein the combination of R² and R³ is Y-phenyl-Z.

31. The compound of claim 30, wherein said compound is represented by the structure of formula (23):

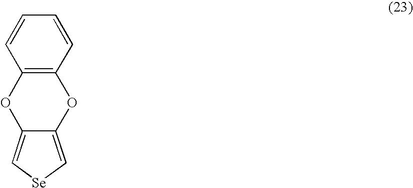

32. The compound of claim 1, wherein said compound is represented by the structure of formula (24):

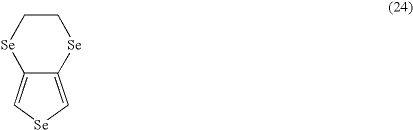

33. A polymer represented by formula (25):
-[(A)ₒ-(B)ₚ—(C)_{q}]ᵣ— (25)
wherein A, B and C may be positioned in any order relative to one another such that A, B and C are either uniformly distributed as blocks throughout the polymer or A, B and C are randomly distributed throughout the polymer and wherein:
A is a monomer unit represented by the structure: 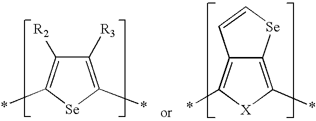
B is a monomer unit comprised of a monocyclic or bicyclic aryl or heteroaryl group wherein said monocyclic or bicyclic aryl or heteroaryl group is optionally substituted with 1-3 groups comprising CN, COOH, C₁-C₁₈ alkyl, OH, O—(C₁-C₁₈ alkyl), SH, S—(C₁-C₁₈ alkyl), NH₂, NH—(C₁-C₁₈ alkyl), or N(C₁-C₁₈ alkyl)₂;
C is a monomer unit comprised of a substituted or unsubstituted vinyl or acetylene group;
o is an integer from 1-10,000;
p is an integer from 0-10,000;
q is an integer from 0-10,000;
r is an integer from 2-10,000;
R² is H, F, Cl, Br, I, C₁-C₆alkyl, S, O, NH, Y—H or Y—(C₁-C₆ alkyl) wherein if R³ is H then R² is not C₁-C₆ alkyl;
R³ is H, F, Cl, Br, I, C₁-C₆ alkyl, Z—H or Z—(C₁-C₆ alkyl) wherein if R² is H then R³ is not C₁-C₆ alkyl;
or
R² and R³ combine to form a 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];
or
R² and R³ combine to form Y-ring-Z having the following structure; 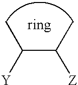
wherein said ring of Y-ring-Z is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];
R⁴ is C₁-C₆ alkyl;
R⁵ is C₁-C₆ alkyl;
Y is O, S, Se, NR⁶ and C(R⁷)(R⁸);
Z is O, S, Se, NR⁹ and C(R¹⁰)(R¹¹);
R⁶ is H, C₁-C₆ alkyl or C(O)(C₁-C₆ alkyl);
R⁷ is H, CN, C₁-C₆ alkyl, OH, SH, NH₂ or aryl;
R⁸ is H, CN, C₁-C₆ alkyl, OH, SH, NH₂ or aryl;
R⁹ is H, C₁-C₆ alkyl or C(O)(C₁-C₆ alkyl);
R¹⁰ is H, CN, C₁-C₆ alkyl, OH, SH, NH₂ or aryl; and
R¹¹ is H, CN, C₁-C₆ alkyl, OH, SH, NH₂ or aryl;
X is S, Se.

34. The polymer of claim 33, wherein p is 0.

35. The polymer of claim 33, wherein q is 0.

36. The polymer of claim 33, wherein p and q are 0.

37. The polymer of claim 33, wherein p and q are 0 and A is represented by the structure of formula (26):

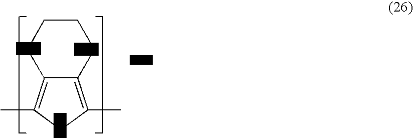

38. The polymer of claim 33, wherein p and q are 0 and A is represented by the structure of formula (27):

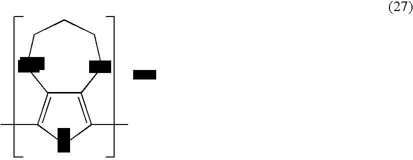

39. The polymer of claim 33, wherein p and q are 0 and A is represented by the structure of formula (28):

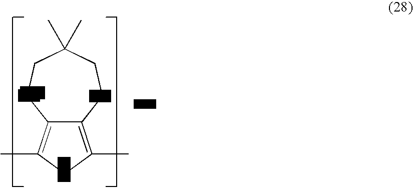

40. The polymer of claim 33, wherein p and q are 0 and A is represented by the structure of formula (31):

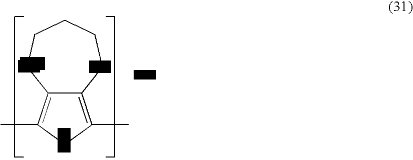

41. The polymer of claim 33, wherein p and q are 0 and A is represented by the structure of formula (32):

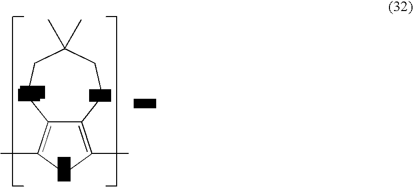

42. The polymer of claim 33, wherein p and q are 0 and A is represented by the structure of formula (35):

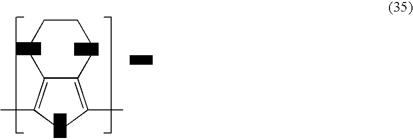

43. The polymer of claim 33, wherein p and q are 0 and A is represented by the structure of formula (36):

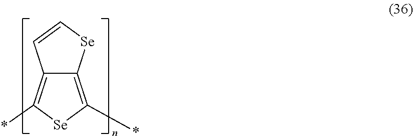

44. The polymer of claim 33, wherein the monomer unit A is represented by the structure of formula (37):

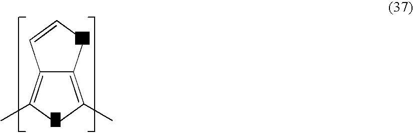

45. The polymer of claim 33, wherein at least one of p or q is not zero and A is represented by the structure of formula (38):

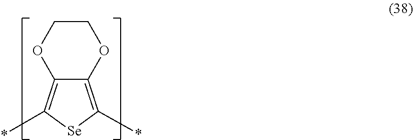

46. The polymer of claim 33, wherein B is a thiophene ring.

47. The polymer of claim 33, wherein B is a phenyl ring.

48. A process for preparing a compound represented by the structure of formula (1): 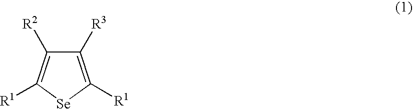
wherein:
R¹ is H, F, Cl, Br, I, SH, OSO₂CH₃ or OSO₂CF₃;
R² is H, F, Cl, Br, I, C₁-C₆alkyl, S, O, NH, Y—H or Y—(C₁-C₆ alkyl) wherein if R³ is H then R² is not C₁-C₆ alkyl;
R³ is H, F, Cl, Br, I, C₁-C₆ alkyl, Z—H or Z—(C₁-C₆ alkyl) wherein if R² is H then R³ is not C₁-C₆ alkyl;
or
R² and R³ combine to form a substituted or unsubstituted 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];
or
R² and R³ combine to form Y-ring-Z having the following structure; 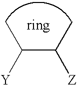
wherein said ring of Y-ring-Z is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];
R⁴ is C₁-C₆ alkyl;
R⁵ is C₁-C₆ alkyl;
Y is O, S, NR⁶ and C(R⁷)(R⁸);
Z is O, S, NR⁹ and C(R¹⁰)(R¹¹);
R⁶ is H, C₁-C₆ alkyl or C(O)(C₁-C₆ alkyl);
R⁷ is H, CN, C₁-C₆ alkyl, OH, SH, NH₂ or aryl;
R⁸ is H, CN, C₁-C₆ alkyl, OH, SH, NH₂ or aryl;
R⁹ is H, C₁-C₆ alkyl or C(O)(C₁-C₆ alkyl);
R¹⁰ is H, CN, C₁-C₆ alkyl, OH, SH, NH₂ or aryl;
R¹¹ is H, CN, C₁-C₆ alkyl, OH, SH, NH₂ or aryl;
said process comprising the steps of:
c) reacting 3,4-dimethoxyselenophene with a nucleophile to yield 3,4-substituted-selenophene: 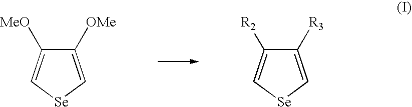
d) optionally brominating 3,4-substituted-selenophene (1) of step (a), obtaining 2,5 dibromoselenophene: 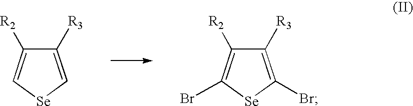
and
e) optionally substituting the bromide of compound (II) of step (b) with a nucleophile, obtaining the compound of formula (1).

49. The process of claim 48, wherein 3,4-dimethoxyselenophene is prepared by condensation of 2,3-dimethoxybutadiene with selenium dichloride.

50. The process of claim 48, wherein step (a) is catalyzed by an acid.

51. The process of claim 50 wherein said acid is para-toluenesulfonic acid.

52. The process of claim 48, wherein said nucleophile of step (a) is Y-ring-Z, Y—H, Y—(C₁-C₆ alkyl), ethane-1,2-diol, propane-1,3-diol, ethane-1,2-dithiane, propane-1,2-dithiane, 2-mercapto-ethanol, or 3-mercapto-propanol wherein said uncleophile is optionally substituted or unsubstituted.

53. The process of claim 48, wherein said brominating step comprises a brominating agent.

54. The process of claim 53, wherein said brominating agent is bromine.

55. The process of claim 53, wherein said brominating agent is N-bromosuccinimide.

56. A process for preparing a polymer of formula (25):
-[(A)ₒ-(B)ₚ—(C)_{q}]ᵣ— (25)
wherein A, B and C may be positioned in any order relative to one another such that A, B and C are either uniformly distributed as blocks throughout the polymer or A, B and C are randomly distributed throughout the polymer and wherein:
A is a monomer unit represented by the structure: 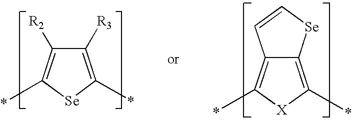
B is a monomer unit comprised of a monocyclic or bicyclic aryl or heteroaryl group wherein said monocyclic or bicyclic aryl or heteroaryl group is optionally substituted with 1-3 groups comprising CN, COOH, C₁-C₁₈ alkyl, OH, O—(C₁-C₁₈ alkyl), SH, S—(C₁-C₁₈ alkyl), NH₂, NH—(C₁-C₁₈ alkyl), or N(C₁-C₁₈ alkyl)₂;
C is a monomer unit comprised of a substituted or unsubstituted vinyl or acetylene group;
o is an integer from 1-10,000;
p is an integer from 0-10,000;
q is an integer from 0-10,000;
r is an integer from 2-10,000;
R² is H, F, Cl, Br, I, C₁-C₆alkyl, S, O, NH, Y—H and Y—(C₁-C₆ alkyl) wherein if R³ is H then R² is not C₁-C₆ alkyl;
R³ is H, F, Cl, Br, I, C₁-C₆ alkyl, Z-H and Z-(C₁-C₆ alkyl) wherein if R² is H then R³ is not C₁-C₆ alkyl;
or
R² and R³ combine to form a 4-8 membered ring comprising 0-3 double bonds and 0-3 heteroatoms selected from O, N, Se or S wherein said 4-8 membered ring is optionally substituted with 1-3 groups comprising C₁-C₁₂ alkyl, (C₀-C₆ alkyl)-cycloalkyl, (C₀-C₆ alkyl)-aryl, (C₀-C₆ alkyl)-heteroaryl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)]; wherein said aryl, cycloalkyl and heteroaryl groups of said (C₀-C₆ alkyl)-aryl, (C₀-C₆ alkyl)-cycloalkyl and (C₀-C₆ alkyl)-heteroaryl groups are optionally substituted with 1-3 groups comprising halide, C₁-C₆ alkyl, CN, CO₂H, OH, SH, NH₂, CO₂—(C₁-C₆ alkyl), O—(C₁-C₆ alkyl), S—(C₁-C₆ alkyl), NH(C₁-C₆ alkyl), N(R₄)(R₅), NHC(O)(C₁-C₆ alkyl) or N[(C₁-C₆ alkyl)][C(O)(C₁-C₆ alkyl)];
or
R² and R³ combine to form Y-ring-Z having the following structure; 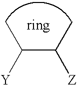
wherein said ring of Y-ring-Z is a cycloalkyl, heterocycloalkyl or aryl optionally substituted by 1-3 groups comprising halide, CN, CO₂H OH, SH, NH₂, CO₂—(C₁-C₆ alkyl), O—(C₁-C₆ alkyl), S—(C₁-C₆ alkyl), NH(C₁-C₆ alkyl), N(R⁴)(R⁵), NHC(O)(C₁-C₆ alkyl) or N[(C₁-C₆ alkyl)][C(O)(C₁-C₆ alkyl)];
R⁴ is C₁-C₆ alkyl;
R⁵ is C₁-C₆ alkyl;
Y is O, S, Se, NR⁶ or C(R⁷)(R⁸);
Z is O, S, Se, NR⁹ or C(R¹⁰)(R¹¹);
R⁶ is H, C₁-C₆ alkyl or C(O)(C₁-C₆ alkyl);
R⁷ is H, CN, C₁-C₆ alkyl, OH, SH, NH₂ or aryl;
R⁸ is H, CN, C₁-C₆ alkyl, OH, SH, NH₂ or aryl;
R⁹ is H, C₁-C₆ alkyl or C(O)(C₁-C₆ alkyl);
R¹⁰ is H, CN, C₁-C₆ alkyl, OH, SH, NH₂ or aryl; and
R¹¹ is H, CN, C₁-C₆ alkyl, OH, SH, NH₂ or aryl;
X is S or Se;
said process comprises the step of polymerizing a 2,5-dibromoselenophene or 2,5-diiodoselenophene monomer unit of A with monomer unit B, monomer unit C or any combination thereof, in the presence of Ni(COD)₂ or by heating said monomer units to a temperature range of between 20-150° C.; or said process comprises the step of polymerizing a selenophene monomer unit of A, with monomer unit B, monomer unit C or any combination thereof, in the presence of FeCl₃ or polymerizing said monomers electrochemically, wherein position 2,5 of said selenophene monomer unit of A are hydrogens.

57. The process of claim 56, wherein p and q are 0 and said monomer A is represented by the structure of formula (38):

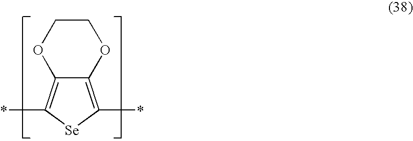

58. The process of claim 56, wherein p and q are 0 and said monomer A is represented by the structure of formula (31):

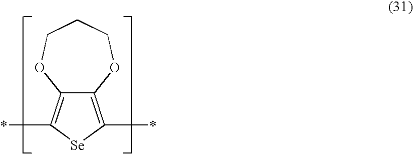

59. The process of claim 56, wherein p and q are 0 and said monomer A is represented by the structure of formula (32):

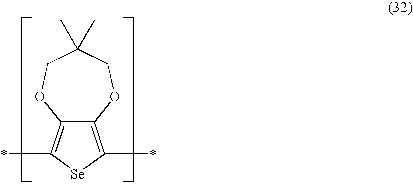

60. The process of claim 56, wherein p and q are 0 and said monomer A is represented by the structure of formula (40): 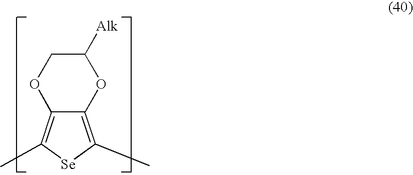
wherein Alk is a C₁-C₁₂ alkyl.

61. The process of claim 61, wherein said alkyl is ethyl, butyl, hexyl or dodecyl.

62. The process of claim 56, wherein p and q are 0 and said monomer A is represented by the structure of formula (26):

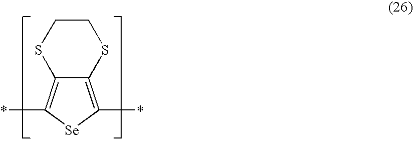

63. The process of claim 56, wherein p and q are 0 and said monomer A is represented by the structure of formula (27):

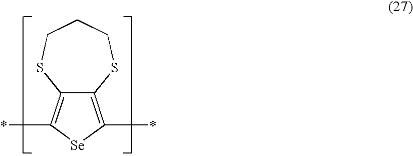

64. The process of claim 67, wherein p and q are 0 and said monomer A is represented by the structure of formula (28):

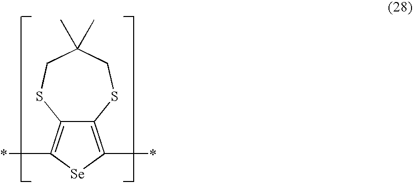

65. A polymer of claim 33, prepared by polymerizing a monomer unit A, monomer unit B, monomer unit C or any combination thereof by heating said monomer to a temperature range of between 30-90° C.

66. The polymer of claim 65, wherein said polymer is a conductive polymer having a conductivity of about 10 S·cm⁻¹.

67. The polymer of claim 33, wherein said polymer is doped.

68. The polymer of claim 67 wherein said doping is p-type.

69. The polymer of claim 68 wherein the dopant is Br₃⁻, I₃⁻, AsF₆⁻, ClO₄⁻, BF₄⁻ or FeCl₄⁻.

70. The polymer of claim 69 wherein the dopant is Br₃⁻.

71. The polymer of claim 67 wherein said doping is n-type.

72. The polymer of claim 71 wherein the dopant is Li⁺, Na⁺ or K⁺.

73. An electronic device comprising a layer obtained from the conducting polymer of claim 33.

74. The electronic device of claim 73, wherein said device is an organic light-emitting device, wherein the layer is at least one of a hole injecting layer and a hole transporting layer.

75. The electronic device of claim 74, wherein said electronic device is a photovoltaic device, an electrochromic device, an electrophoretic device, an organic thin film transistor, or an organic memory device.

76. An organic light-emitting device, comprising:
a first electrode;
a second electrode;
an emitting layer interposed between the first electrode and the second electrode; and at least one of a hole transporting layer and a hole injecting layer interposed between the emitting layer and the first electrode, said at least one of the hole transporting layer and the hole injecting layer obtained from a conducting polymer of claim 33.

77. An electrochromic device comprising a conducting polymer of claim 33 wherein said conducting polymer has a high coloration efficiency.

78. A radialene compound of formula (46): 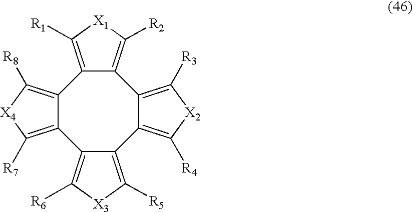
wherein:
R¹, R², R³, R⁴, R⁵, R⁶, R⁷ or R⁸ are independently H, F, Cl, Br, I, CN, OH, SH, NH₂, O—(C₁-C₆ alkyl), S—(C₁-C₆ alkyl), NH(C₁-C₆ alkyl), N(R⁹)(R¹⁰), NHC(O)(C₁-C₆ alkyl) and N[(C₁-C₆ alkyl)][C(O)(C₁-C₆ alkyl)] or C(O)OR¹¹;
R⁹ is C₁-C₆ alkyl;
R¹⁰ is C₁-C₆alkyl;
R¹¹ is C₁-C₆ alkyl; and
X¹, X², X³ or X⁴ are independently O, S, Se, Te, NH or PH;
wherein X¹, X², X³ and X⁴ are not S.

79. A radialene compound of formula (47): 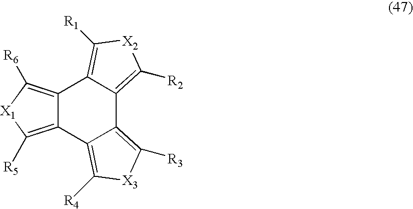
wherein:
R¹, R², R³, R⁴, R⁵ or R⁶ are independently H, F, Cl, Br, I, CN, OH, SH, NH₂, O—(C₁-C₆ alkyl), S—(C₁-C₆ alkyl), NH(C₁-C₆ alkyl), N(R⁷)(R⁸), NHC(O)(C₁-C₆ alkyl) and N[(C₁-C₆ alkyl)][C(O)(C₁-C₆ alkyl)] or C(O)OR⁹;
R⁷ is C₁-C₆alkyl;
R⁸ is C₁-C₆ alkyl;
R⁹ is C₁-C₆ alkyl;
X¹, X² or X³ are independently O, S, Se, Te, NH or PH;
wherein X¹, X² and X³ are not S.

80. A polyselenophene dispersion comprising positively charged polyselenophenes and anions, wherein said polyselenophene is of claim and said anion is tosylate, acrylate, maleate, sulfonate, p-toluenesulfate, 4-ethlybenzenesulfonate, camphor-sulfonate, tetradecyl-sulfonate, dodecyl-sulfonate, methane-sulfonate, naphthalene sulfonate, triflate, or any combination thereof; or an anion of polyacrylic acid, polymethacrylic acid, polymaleic acid, polystyrene sulfonic acid, polyvinyl sulfonic acid combination thereof.

81. The dispersion according to claim 80, wherein said dispersion is poly(3,4-ethylenedioxyselenophene) and poly(styrene sulfonate)—PEDOS:PSS.

82. The dispersion of claim 80, wherein said dispersion is in water, alcohol or combination thereof.

83. A transparent conductive electrode comprising a polyselenophene dispersion of claim 80.
